(19) United States
(12) Patent Application Publication
(10) Pub. No.: US 2025/0011418 A1
(43) Pub. Date: Jan. 9, 2025

(54) CANCER SPECIFIC PLECTIN-1 SPECIFIC ANTIBODIES AND METHODS OF USE THEREOF

(71) Applicant: ZielBio, Inc., Charlottesville, VA (US)

(72) Inventors: Kimberly KELLY, Charlottesville, VA (US); Randall BURTON, Charlottesville, VA (US); Arnold HORWITZ, Charlottesville, VA (US)

(21) Appl. No.: 18/705,022

(22) PCT Filed: Oct. 28, 2022
(86) PCT No.: PCT/US2022/078928

§ 371 Date: Apr. 26, 2024

Related U.S. Application Data
(60) Provisional application No. 63/310,824, filed on Feb. 16, 2022.
(60) Provisional application No. 63/273,446, filed on Oct. 29, 2021.

(51) Int. Cl. C07K 16/28; A61K 39/00; A61K 47/68; A61P 35/00

(52) U.S. Cl. CPC C07K 16/28; A61K 47/68037; A61K 47/6849; A61P 35/00; A61K 2039/505; C07K 2317/24; C07K 2317/52; C07K 2317/565; C07K 2317/569; C07K 2317/73; C07K 2317/77; C07K 2317/92

(57) ABSTRACT

The disclosure provides immunotherapeutic approaches to effectively and specifically treat cancer by targeting cancer cells overexpressing cancer specific plectin-1 (CSP-1). In some embodiments, the disclosure provides antibodies, or antigen binding fragments thereof, that bind CSP-1.

### CROSS REFERENCE TO RELATED APPLICATIONS

The present application claims the benefit of priority to U.S. Provisional Application No. 63/273,446 filed on Oct. 29, 2021, and U.S. Provisional Application No. 63/310,824 filed Feb. 16, 2022, the contents of each of which are hereby incorporated by reference in their entireties for all purposes.

### REFERENCE TO AN ELECTRONIC SEQUENCE LISTING

The contents of the electronic sequence listing (ZIEL_006_02WO_SeqList_ST26.xml; Size: 529,384 bytes; and Date of Creation: Oct. 28, 2022) are herein incorporated by reference in its entirety.

### BACKGROUND

Cancer is among the leading causes of death worldwide. In 2020, an estimated 1,806,590 new cases of cancer were diagnosed in the United States and 606,520 people died from the disease. Cancer is a complex disease that involves a sequence of gene-environment interactions and/or dysfunction in multiple systems, including DNA repair, apoptotic and immune functions.

Given the heterogeneous and dynamic nature of cancer, there has been considerable focus on identifying and targeting cancer biomarkers. Cancer biomarkers are biological molecules (such as, proteins, miRNA), which are associated with, and often indicative of, the presence of cancer in an individual. Identifying cancer biomarkers in the individual can provide insight into which therapy is most likely to work, and the risk of cancer recurrence. Furthermore, targeting cancer biomarkers using drugs or biologics can also help treat some types of cancers.

There continues to be a need to identify and target new cancer biomarkers that can be used for the diagnosis and treatment of cancers, particularly across different cancer types.

### SUMMARY

The disclosure provides antibodies or antigen-binding fragment thereof that binds cancer specific plectin-1 (CSP-1). In some embodiments, the antibody or antigen-binding fragment thereof that binds cancer specific plectin-1 (CSP-1), comprises a heavy chain variable region, and a light chain variable region, wherein the heavy chain variable region comprises a heavy chain complementary determining region 1 (HCDR1), a HCDR2, and a HCDR3 and wherein the light chain variable region comprises a light chain complementary determining region 1 (LCDR1), a LCDR2, and a LCDR3, wherein the HCDR1 comprises an amino acid sequence according to SEQ ID NO: 104 (GFTFSRYG), the HCDR2 comprises an amino acid sequence according to SEQ ID NO: 115 (ISIGGTYT), the HCDR3 comprises an amino acid sequence according to SEQ ID NO: 134 (ARRGYGX₅YSYYGX₁₁DY, wherein X₅ is N, S, or Q; and X₁₁ is M, L, I, or V); the LCDR1 comprises an amino acid sequence according to SEQ ID NO: 135 (KSLLHSX₁₀GITY, wherein X₁₀ is N, V, D, Q, S or E); the LCDR2 comprises an amino acid sequence according to SEQ ID NO: 136 (QX₂S, wherein X₂ is M, L, V, or I); and the LCDR3 comprises an amino acid sequence according to SEQ ID NO: 133 (AQNLELPLT).

In some embodiments, the antibody or antigen binding fragment comprises a heavy chain variable region having at least about 90% identity to SEQ ID NO: 51. In some embodiments, the antibody or fragment comprises a light chain variable region having at least about 90% identity to SEQ ID NO: 102. In some embodiments, the antibody or fragment comprises a heavy chain amino acid sequence according to SEQ ID NO: 186 and a light chain amino acid sequence according to SEQ ID NO: 187.

The disclosure further provides methods for treating a cancer comprising administering to a subject in need thereof any one of the antibodies or antigen binding fragments thereof disclosed herein. The disclosure further provides methods for inducing immune-related cell death of cancer cells in a subject having cancer comprising administering to the subject any one of the antibodies or antigen binding fragments thereof disclosed herein. In some embodiments, the cancer is selected from the group consisting of pancreatic cancer, ovarian cancer, gastric cancer, colorectal carcinoma, cholangiocarcinoma, Head and neck squamous cell carcinoma (HNSCC), triple negative breast cancer (TNBR), prostate cancer, and non-small cell lung cancer (NSCLC). In some embodiments, the immune-related cell death induced by the administration of the antibody or antigen binding fragment thereof increases anti-cancer T cell responses in the subject. In some embodiments, the administration of the antibody or antigen binding fragment thereof elicits at least a 2-fold increase in effector memory T cells in the subject. In some embodiments, the administration of the antibody or antigen binding fragment thereof increases macrophage infiltration of tumors in the subject.

These and other embodiments are addressed in more detail in the detailed description set forth below.

### BRIEF DESCRIPTION OF THE FIGURES

FIG. 1 is a graph showing results from an ELISA assay to measure the binding of Sec8-His to the humanized anti-CSP-1 antibody, Ab51.

FIG. 2 shows the results from bio-layer interferometry analysis of binding between humanized anti-CSP-1 antibody, Ab51 and Sec8-His.

FIG. 3A is a graph showing the average tumor volume over time in a mouse model of ovarian cancer treated with the humanized anti-CSP-1 antibody, Ab51 or a control IgG antibody or murine antibody, 1H11B5. FIG. 3B is a graph showing the average tumor volume over time in a syngeneic mouse model of KPC915-derived pancreatic cancer treated with murine anti-CSP-1 antibody (1D7) or control IgG antibody. FIG. 3C is a graph showing the average tumor volume over time in a syngeneic mouse model of KPC915-derived pancreatic cancer treated with murine anti-CSP-1 antibody (1D7) or control IgG antibody.

FIG. 4A is a bar graph showing the fold change of relative phosphorylation status of the indicated stress-related proteins in OVCAR8 cells treated for 10 mins with 125 nM of murine anti-CSP-1 antibody (1H11) or control IgG antibody. FIG. 4B shows an image from a Western Blot experiment showing protein bands of phosphorylated HSP27, unphosphorylated HSP-27, and control GAPDH protein from extracts of OVCAR8 cells after treatment with 125 nM murine anti-CSP-1 antibody (1H11), or the control IgG antibody. FIG. 4C shows the quantitation of the protein band signal from the Western Blot image of FIG. 4B.

FIG. 5A shows a bar graph of the amount of reactive oxygen species (ROS) detected in ovarian cancer OVCAR8 cells after treatment with a 2 uM of control IgG antibody or a humanized anti-CSP-1 antibody, Ab51. FIG. 5B shows a bar graph of the amount of reactive oxygen species (ROS) detected in ovarian cancer OVCAR8 cells after treatment with different concentrations of cisplatin.

FIG. 6A shows an image from a Western Blot experiment showing protein bands of the indicated proteins from extracts of KPC915 cancer cells after treatment for 24 hours with 500 nM murine anti-CSP-1 antibody (1D7), or the control IgG antibody. FIGS. 6B-6E show the quantitation of the protein band signal from the Western Blot image of FIG. 6A for pro-caspase (FIG. 6B), cleaved caspase 1 (FIG. 6C), cleaved N-terminal region of gasdermin D (GSDMD; FIG. 6D) and cleaved C-terminal region of gasdermin (FIG. 6E).

FIG. 7A shows a bar graph of fold change in the amount of IL-18 secreted in the supernatant of OVCAR8 cells upon treatment with the indicated concentrations of Ab51 relative to the control antibody. FIG. 7B shows an image from a Western Blot experiment showing protein bands of IL-1β from extracts of KPC915 cancer cells after treatment with 500 nM murine anti-CSP-1 antibody, 1D7, or the control IgG antibody. FIG. 7C shows the quantitation of the protein band signal from the Western Blot image of FIG. 7B.

FIG. 8A shows the distribution of the immune cell types in pancreatic ductal adenocarcinoma (PDAC) tumors isolated from mice treated with one or two doses of murine anti-CSP-1 antibody (1H11), or a control IgG antibody. FIG. 8B shows pie charts showing the distribution of the different subtypes of CD8+ T cells in the PDAC tumors isolated from mice treated with one or two doses of murine anti-CSP-1 antibody (1H11), or a control IgG antibody. FIG. 8C shows images of histology of PDAC tumor tissue from mice treated with one or two doses of murine anti-CSP-1 antibody (1H11), or a control IgG antibody.

FIG. 9 is a graph showing the average tumor volume over time in nu/J mice that lack T-cells and have a partial defect in B-cells, which are implanted with OVCAR8 ovarian cancer cells and treated with 5 mg/kg anti-CSP-1 antibody 1H11 or control IgG antibody twice a week.

FIG. 10A shows images from immunohistochemistry using marker F4/80, a glycoprotein expressed by murine macrophages, of tumor tissue from Ab51-treated mice, murine anti-CSP-1 antibody-treated mice (1H11), or control IgG antibody-treated mice. FIG. 10B shows the quantitation of 3,3′-diaminobenzidine (DAB)-positive cells in tumor tissue based on the immunohistochemistry analysis.

FIG. 11A is a graph showing the growth of individual tumors from extrahepatic cholangiocarcinoma (ECC) WITT cells over time in mice treated with the humanized anti-CSP-1 antibody, Ab51, or a control IgG antibody. FIG. 11B is a bar graph showing the % of dendritic cells in tumors isolated from the IgG-treated mice or Ab51-treated mice at 11 days. FIG. 11C is a bar graph showing the % of macrophages in tumors isolated from the IgG-treated mice or Ab51-treated mice at 11 days.

FIG. 12A is a graph showing the average tumor volume from ovarian cancer cell line (OVCAR8) over time in female NMRI/nu murine model treated with the humanized anti-CSP-1 antibody, Ab51, a control IgG antibody, or vehicle control. FIG. 12B shows images of tumor tissue showing immunohistochemical staining of Ki67 or Cyclin D proliferation markers. The tumor tissue was isolated from murine OVCAR8 xenograft models treated with Ab51, a murine anti-CSP-1 antibody (1H11) or a control IgG antibody. FIG. 12C shows the quantitation of immunohistochemical staining of Ki67 or Cyclin D.

FIG. 13A depicts the results from a flow cytometry experiment on untreated YAPC pancreas carcinoma cells, or YAPC pancreas carcinoma cells treated with 500 nM of 1H11. FIG. 13B is a bar graph showing the proportion of ovarian cancer cell line OVCAR8 in different cell cycle stages (G0/G1, S or G2/M) upon treatment with Ab51 antibody or a control IgG antibody.

FIG. 14A shows an image from a Western Blot experiment showing protein bands of the indicated proteins from extracts of OVCAR8 cancer cells after treatment with 125 nM murine anti-CSP-1 antibody (1H11), or the control IgG antibody. FIGS. 14B-14F show the quantitation of the protein band signal from the Western Blot image of FIG. 14A for phosphorylated cyclin D1 (FIG. 14B), cyclin D1 (FIG. 14C), CDK6 (FIG. 14D), p27 (FIG. 14E), and p21 (FIG. 14F). FIG. 14G shows an image from a Western Blot experiment showing protein bands of β-catenin from extracts of cancer cells after treatment with 125 nM murine anti-CSP-1 antibody (1H11), or the control IgG antibody. FIG. 14H shows the quantitation of the β-catenin protein band signal from the Western Blot image of FIG. 14G.

FIG. 15A shows images showing p21 from histology of tumor tissues isolated from mice treated with either the murine anti-CSP-1 antibody (1H11) or the control IgG antibody. FIG. 15B shows the quantitation of the p21 signal-positive cells based on the histology images of FIG. 15A.

FIG. 16A is a bar graph showing the fold change of relative phosphorylation of the indicated Akt/PRAS40 pathway proteins in OVCAR8 cells after treatment for 10 mins with 125 nM of either control IgG antibody or murine anti-CSP-1 antibody (1H11). FIG. 16B shows images showing phosphorylated AKT (pAKT) from histology of MiaPACA2 tumor tissue isolated from mice treated with murine anti-CSP-1 antibody (1H11) or control IgG antibody. FIG. 16C shows the quantitation of the pAKT signal-positive cells based on the histology images of FIG. 16B.

FIG. 17A shows images from scratch assay of cholangiocarcinoma cell line HuCCT1 treated with vehicle control, control IgG antibody, a murine CSP-1 antibody (1D7) or Ab51. FIG. 17B is a bar graph showing the percentage of open wound in cell culture treated with vehicle control, control IgG antibody, a murine CSP-1 antibody or Ab51 over time.

FIG. 18A shows images from scratch assay of ovarian cancer cell line SKOV3 treated with vehicle control, control IgG antibody, a murine CSP-1 antibody (1H11) or Ab51. FIG. 18B is a bar graph showing the percentage of open wound in cell culture treated with vehicle control, control IgG antibody, a murine CSP-1 antibody (1H11) or Ab51 over time. FIG. 18C shows the results from a Western Blot evaluating the levels of E-cadherin and vimentin in cells treated with Ab51, or control IgG antibody, and the quantitation thereof in the form of bar graphs.

FIG. 19A shows images from flow cytometry of ovarian cancer cells OVCAR8 treated with murine anti-CSP-1 antibody (1H11) or control IgG antibody to assess the proportion of apoptotic cells in the population over time. Propidium iodide (PI) and annexin V were used as markers of apoptosis. FIG. 19B shows the quantitation of % apoptotic cells in OVCAR8 cells treated with murine anti-CSP-1 antibody (1H11) or control IgG antibody based on the flow cytometry results.

FIG. 19C is a bar graph showing the activity of caspase 3 and caspase 7 in OVCAR8 cells treated with murine anti-CSP-1 antibody (1H11), control IgG antibody, or paclitaxel. FIG. 19D shows images from histology of tumor tissue from immunocompromised mice with MIAPACA-2 tumors in nu/nu background treated with 1H11 murine anti-CSP-1 antibody, or control IgG antibody. Staining for pERK1/2 gives a measure of cell proliferation. FIG. 19E shows the quantification of the caspase 3 signal-positive cells based on the histology images of FIG. 19D.

FIG. 20A is a graph showing the average growth of tumors from extrahepatic cholangiocarcinoma (ECC) WITT cells over time in mice with defective immune system treated with the humanized anti-CSP-1 antibody, Ab51, or a control IgG antibody. FIG. 20B is a graph showing the average growth of tumors from MiaPACA-2 cells over time in immunodeficient mice treated with murine anti-CSP-1 antibody (1H11) or the control IgG antibody.

FIG. 21A is a graph showing the % complement cytotoxicity upon treatment of OVCAR8 cells with the indicated antibodies. FIG. 21B is a graph showing the complement cytotoxicity upon treatment of Raji cells with anti-CD20 antibody or a control IgG antibody.

FIG. 22A is a graph showing the % target cell cytotoxicity in Raji cells upon treatment with different concentrations of the indicated antibodies. FIG. 22B is a graph showing the % target cell cytotoxicity in OVCAR8 cells upon treatment with different concentrations of the indicated antibodies.

FIG. 23A is a graph showing the results from an ELISA assay performed to evaluate the binding of the humanized anti-CSP-1 antibody, Ab51 to Reb-His peptide or a control GST-His peptide. FIG. 23B is a graph showing results from an ELISA assay performed to evaluate the binding of the murine anti-CSP-1 antibody, 1H11 to Reb-His peptide or a control GST-His peptide.

FIG. 23C is a graph showing results from an ELISA assay performed to evaluate the binding of the murine anti-CSP-1 antibody, 1D7 to Reb-His peptide or a control GST-His peptide.

FIG. 24 is a graph showing results from an ELISA assay performed to evaluate the binding to Sec8-His of: (i) the murine anti-CSP-1 antibody, 1H11 or (ii) an antibody-drug conjugate comprising 1H11 and a chemotherapeutic drug, SN38.

FIG. 25A is a graph showing the percentage of live OVCAR8 ovarian cancer cells detected after treatment with varying concentrations of 1H11 (on the X axis) normalized to the percentage of live OVCAR8 ovarian cancer cells detected after treatment with a control vehicle. FIG. 25B is a graph showing the percentage of live OVCAR8 ovarian cancer cells detected after treatment with varying concentrations of an antibody-drug conjugate of 1H11 and SN38 (on the X axis) normalized to the percentage of live OVCAR8 ovarian cancer cells detected after treatment with a control vehicle. FIG. 25C is a graph showing the percentage of live MIA PaCa-1 pancreatic cancer cells detected after treatment with varying concentrations of an antibody-drug conjugate of 1H11 and SN38 (on the X axis) normalized to the percentage of live MIA PaCa-1 pancreatic cancer cells detected after treatment with a control vehicle.

FIG. 26A is a graph showing the percentage of live OVCAR8 ovarian cancer cells detected after treatment with varying concentrations of an antibody-drug conjugate of 1H11 and SN38, or SN38 alone (on the X axis) normalized to the percentage of live OVCAR8 ovarian cancer cells detected after treatment with a control vehicle. FIG. 26B is a graph showing the percentage of live MIA PaCa-2 pancreatic cancer cells detected after treatment with varying concentrations of an antibody-drug conjugate of 1H11 and SN38, or SN38 alone (on the X axis) normalized to the percentage of live MIA PaCa-2 pancreatic cancer cells detected after treatment with a control vehicle.

FIG. 27 is a bar graph showing the normalized fluorescence intensity in the cell types indicated on the X axis, measured 24 hours after injection, into a syngeneic mouse model of ductal adenocarcinoma of the pancreas (PDAC), of: (i) a Alexa Fluor 750 (AF750) dye labelled reverse chimeric antibody (comprising the complementarity determining regions (CDRs) of Ab51 on a murine IgG2a constant region), or (ii) a AF750 dye-labeled control murine IgG2a antibody.

FIG. 28A shows an image from immunohistochemistry experiments using xenografted tumor tissue from nude mice intravenously injected with control IgG1 antibody. FIG. 28B shows an image from immunohistochemistry experiments using xenografted tumor tissue from nude mice tissue intravenously injected with 5 mg/kg of the humanized anti-CSP-1 antibody, Ab51. The presence of Ab51 was detected in the tumor tissue. See Example 13 for details.

### DETAILED DESCRIPTION

Cancer specific plectin-1 (CSP-1) is a cell-surface localized cancer protein biomarker. CSP-1 is overexpressed by the cancer cells of a majority of patients with different types of cancers, such as, cholangiocarcinoma, pancreatic cancer, ovarian cancer, gastric cancer, colorectal carcinoma, esophageal squamous cell carcinoma (SCC), head and neck squamous cell carcinomas (HNSCC), triple negative breast cancer (TNBR), prostate cancer and non-small cell lung cancer (NSCLC). In particular, CSP-1 is aberrantly expressed on Pancreatic ductal adenocarcinoma (PDAC) cells.

The disclosure provides immunotherapeutic approaches to effectively and specifically treat cancer by targeting cancer cells overexpressing CSP-1. In some embodiments, the disclosure provides antibodies, or antigen binding fragments thereof, that bind CSP-1.

The section headings used herein are for organizational purposes only and are not to be construed as limiting the subject matter described. Unless the context indicates otherwise, it is specifically intended that the various features described herein can be used in any combination. Unless otherwise defined, all technical and scientific terms used herein have the same meaning as commonly understood by one of ordinary skill in the art to which this disclosure belongs.

### Definitions

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

Also as used herein, “and/or” refers to and encompasses any and all possible combinations of one or more of the associated listed items, as well as the lack of combinations when interpreted in the alternative (“or”). The use of the alternative (e.g., “or”) should be understood to mean either one, both, or any combination thereof of the alternatives.

As used herein, the term “cancer specific plectin-1” or “CSP-1” refers to a polypeptide as set forth in SEQ ID NO: 185 or fragments thereof, as well as related polypeptides, which include, but are not limited to, allelic variants, splice variants, derivative variants, substitution variants, deletion variants, and/or insertion variants including the addition of an N-terminal methionine, fusion polypeptides, and interspecies homologs. In certain embodiments, a CSP-1 polypeptide includes terminal residues, such as, but not limited to, leader sequence residues, targeting residues, amino terminal methionine residues, lysine residues, tag residues and/or fusion protein residues. “CSP-1” has also been referred to as EBS1, EBSMD, EBSND, EBSO, EBSOG, EBSPA, HD1, LGMD2Q, LGMDR17, PCN, PLEC1, PLEC1b, and PLT. Further details on CSP-1 are provided in Kelly K A, et al., PLoSMed, 2008, Konkalmatt, et al., Front. Oncol., 2013, Dasa, S S K et al. Theranostics 2018, and Bausch D, et al., Clin Cancer Res. 2011, the contents of each of which are incorporated herein by reference in their entireties. In some embodiments, CSP-1 is encoded by a nucleic acid sequence comprising at least 80% (for example, about 85%, about 90%, about 95%, about 98%, about 99%, or 100%, including all values and subranges that lie therebetween) identity to SEQ ID NO: 184.

As used herein, the term “antigen binding protein” refers to any protein that binds a specified target antigen. The antigen binding proteins disclosed herein bind to the specified target antigen of CSP-1 protein or fragment thereof. The term “antigen binding protein” includes, but is not limited to, antibodies and binding parts thereof, such as “antigen binding fragments” and peptibodies.

As used herein, the term “antibody” refers to an intact immunoglobulin of any isotype, or a fragment thereof that can bind to the target antigen, and includes, for instance, chimeric, humanized, fully human, and bispecific antibodies. In some embodiments, an intact antibody comprises at least two full-length heavy chains and two full-length light chains, but in some embodiments can include fewer chains such as antibodies naturally occurring in camelids which can comprise only heavy chains. The antigen binding proteins, antibodies, or binding fragments may be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, peptibodies, and fragments thereof, respectively.

In some embodiments, definitive delineation of a complementarity determining residue (CDR) of the antibody or antigen binding fragment thereof, and identification of residues comprising the binding site of an antibody is accomplished by solving the structure of the antibody and/or solving the structure of the antibody-ligand complex. In some embodiments, that can be accomplished by any of a variety of techniques known to those skilled in the art, such as X-ray crystallography. In some embodiments, various methods of analysis can be employed to identify or approximate the CDR regions. Examples of such methods include, but are not limited to, the Kabat definition, the Chothia definition, the AbM definition and the contact definition.

The Kabat definition is used for numbering the residues in an antibody and to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

Herein, the CDR regions in the heavy chain may be referred to as HCDR1, HCDR2, and HCDR3 and numbered sequentially in the direction from the amino terminus to the carboxy terminus. The CDR regions in the light chain may be referred to as LCDR1, LCDR2, and LCDR3 and numbered sequentially in the direction from the amino terminus to the carboxy terminus.

The term “antigen binding fragment” (or simply “fragment”) of an antibody or immunoglobulin chain (heavy or light chain) antigen binding protein, as used herein, is a species of antigen binding protein comprising a portion or region of an antibody that lacks at least some of the amino acids present in a full-length chain but which is still capable of specifically binding to the target antigen. In some embodiments, the antigen binding fragments can compete with other antigen binding proteins, including intact antibodies, for binding to a given epitope. In some embodiments, the antigen binding fragments are neutralizing fragments. In some embodiments, the antigen binding fragment comprises at least one “complementary binding regions (CDR) that is present in the full-length light or heavy chain, and in some embodiments will comprise a single heavy chain and/or light chain or portion thereof. In some embodiments, the antigen binding fragments are produced by recombinant DNA techniques, or by enzymatic or chemical cleavage of antigen binding proteins, including intact antibodies.

Immunologically functional immunoglobulin fragments include, but are not limited to, Fab, a diabody (heavy chain variable domain on the same polypeptide as a light chain variable domain, connected via a short peptide linker that is too short to permit pairing between the two domains on the same chain), Fab′, F(ab′)₂, Fv, domain antibodies and single-chain antibodies, and can be derived from any mammalian source, including but not limited to human, mouse, rat, camelid or rabbit. It is further contemplated that a functional portion of the antigen binding proteins disclosed herein, for example, one or more CDRs, could be covalently bound to a second protein or to a small molecule to create a therapeutic or diagnostic agent directed to a particular target in the body, possessing bifunctional therapeutic or diagnostic properties, and/or having a prolonged serum half-life. In some embodiments, an antigen binding protein may include nonprotein components. In some embodiments, the antigen binding fragment comprises or consists of avimers (or avidity multimers).

As used herein, an “Fc” region comprises two heavy chain fragments comprising the C_{H}1 and C_{H}2 domains of an antibody. The two heavy chain fragments are held together by two or more disulfide bonds and by hydrophobic interactions of the C_{H}3 domains.

As used herein, an “Fab fragment” comprises one light chain and the C_{H}1 and variable regions of one heavy chain. The heavy chain of a Fab molecule cannot form a disulfide bond with another heavy chain molecule.

As used herein, an “Fab′ fragment” comprises one light chain and a portion of one heavy chain that contains the VH domain and the C_{H}1 domain and also the region between the C_{H}1 and C_{H}2 domains, such that an interchain disulfide bond can be formed between the two heavy chains of two Fab′ fragments to form an F(ab′)₂ molecule.

As used herein, an “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

As used herein, an “Fv region” comprises the variable regions from both the heavy and light chains, but lacks the constant regions.

As used herein, “single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649 and U.S. Pat. Nos. 4,946,778 and 5,260,203, the contents of which are herein incorporated by reference in their entireties.

As used herein, a “domain antibody” is an immunologically functional immunoglobulin fragment containing only the variable region of a heavy chain or the variable region of a light chain. In some embodiments, two or more V_{H} regions are covalently joined with a peptide linker to create a bivalent domain antibody. The two V_{H} regions of a bivalent domain antibody can target the same or different antigens.

As used herein, a “bivalent antigen binding protein” or “bivalent antibody” comprises two antigen binding sites. In some embodiments, the two binding sites have the same antigen specificities. Bivalent antigen binding proteins and bivalent antibodies can be bispecific.

As used herein, a “multispecific antigen binding protein” or “multispecific antibody” is one that targets more than one antigen or epitope.

As used herein, a “bispecific,” “dual-specific” or “bifunctional” antigen binding protein or antibody is a hybrid antigen binding protein or antibody, respectively, having two different antigen binding sites. Bispecific antigen binding proteins and antibodies may be produced by a variety of methods including, but not limited to, fusion of hybridomas or linking of Fab′ fragments, further details of which are provided in Songsivilai and Lachmann, 1990, Clin. Exp. Immunol. 79:315-321; Kostelny et al., 1992, J. Immunol. 148:1547-1553, the contents of which are herein incorporated by reference in its entirety. In some embodiments, the two binding sites of a bispecific antigen binding protein or antibody bind to two different epitopes residing on the same or different protein targets.

As used herein “sequence identity” refers to the extent to which two optimally aligned polynucleotides or polypeptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in the reference sequence segment, i.e. the entire reference sequence or a smaller defined part of the reference sequence. “Percent identity” is the identity fraction times 100. The extent of identity (homology) between two sequences can be ascertained using a computer program and mathematical algorithm. Percentage identity can be calculated using the alignment program Clustal Omega, available at www.ebi.ac.uk/Tools/msa/clustalo using default parameters. See, Sievers et al., “Fast, scalable generation of high-quality protein multiple sequence alignments using Clustal Omega.” (2011 Oct. 11) Molecular systems biology 7:539. For the purposes of calculating identity to a sequence, extensions such as tags are not included.

In some embodiments, the antibody provided herein is labeled. As used herein, the terms “label” or “labeled” refers to incorporation of a detectable marker, e.g., by incorporation of a radiolabeled amino acid or attachment to a polypeptide of biotin moieties that can be detected by marked avidin (e.g., streptavidin containing a fluorescent marker or enzymatic activity that can be detected by optical or colorimetric methods). In certain embodiments, the label or marker can also be therapeutic. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths to reduce potential steric hindrance. In some embodiments, the present disclosure provides methods for conjugating an antibody provided herein to a chelator, and radiolabeling the conjugated antibody. In some embodiments, the chelator is a standard chelator selected from desferrioxamine (DFO), diethylene triamine pentaacetic acid (DTPA), cyclohexyl-diethylenetriaminepenta acetic acid (CHX-A″-DTPA), 1,2-bis(o-aminophenoxy)ethane-N,N,N′,N′-tetraacetic acid (BAPTA), 3,4,3-(LI-1,2-HOPO) (HOPO), 1,4,7-triazacyclononane-1,4,7-triacetic acid (NOTA), 1,4,7,10-tetraazacyclododecane-1,4,7,10-tetraacetic acid (DOTA), and the like, or any other chelator known in the art. In some embodiments, the radiolabel is 89Zirconium, 86Yttrium, 111Indium, 177Lutetium, or any other diagnostic and/or therapeutic radionuclide known in the art. In some embodiments, the present disclosure comprises a labeled anti-CSP-1 antibody suitable for use in diagnostic applications and/or radioimmunotherapy.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment, such as cancer. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the tissue to be imaged, and the physical delivery system in which it is carried.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant.

As used herein, “antibody-drug conjugate (or ADC)” refers to molecules comprising an antibody, or antigen binding fragment thereof (such as, any of the antibodies or antigen binding fragments disclosed herein) linked to a heterologous moiety (e.g., a biologically active molecule, such as a therapeutic agent, and/or a detectable label). The terms “linked,” “conjugated,” or “coupled” relate to being attached or bound by covalent bonds, or non-covalent bonds, or other bonds, such as van der Waals forces.

As used herein, the term “therapeutic agent” refers to chemicals or drugs or proteins that are able to inhibit cell function, inhibit cell replication or kill cells, such as, human cells. Examples of therapeutic agents include but are not limited to cytotoxic moieties, radioisotopes, molecules of plant, fungal, or bacterial origin (e.g., plant-derived toxins, secondary metabolites), glycosides, antimicrobial compounds (e.g., streptomycin, penicillin, etc.), biological proteins (e.g., protein toxins), particles (e.g., recombinant viral particles, e.g., via a viral coat protein), or mixtures thereof. The therapeutic agent can be an intracellularly active drug or other agent, such as short-range radiation emitters, including, for example, short-131 range, high-energy alpha-emitters (e.g., I).

In some embodiments, the therapeutic agent is an immunomodulatory moiety (e.g., immunomodulatory agent). As used herein, “immunomodulatory agent” refers to a compound or molecule that increases or decreases the immune response of a subject in response to the agent. For example, an immunomodulatory agent may enhance the immune response of a subject to a tumor, e.g., increase the level of inflammatory cytokines such as interleukin-1 (IL-1), and tumor necrosis factor-alpha (TNF-a). Examples of immunomodulatory agents that increase the immune response of a subject include granulocyte colony-stimulating factor (G-CSF), interferons, imiquimod, cellular membrane fractions from bacteria, certain interleukins and cytokines (e.g; IL-Iβ, IL-6, and TNF-a), and immune checkpoint inhibitors (e.g., PD-1 inhibitors, PD1-L inhibitors, etc.). In some embodiments, an immunomodulatory agent may decrease the immune response of a subject (e.g., mediate or achieve immunosuppression). Examples of immunosuppressive immunomodulators include but are not limited to immunosuppressive drugs (e.g., glucocorticoids, cytostatics, anti-inflammatory monoclonal antibodies (e.g., anti-IL-2 receptor antibodies), and drugs targeting immunophilins (e.g., ciclosporin, sirolimus, etc.).

### Anti-CSP-1 Antibodies

The disclosure provides antibodies or antigen-binding fragments thereof that bind cancer specific plectin-1 (CSP-1), comprising a heavy chain variable region, and a light chain variable region. In some embodiments, the heavy chain variable region (VH) of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VH SEQ ID Nos listed in Table 1. In some embodiments, the heavy chain variable region of the antibodies or antigen binding fragments disclosed herein comprises an amino acid sequence having at least about 90% identity to any one of the VH SEQ ID NOs listed in Table 1. In some embodiments, the light chain variable region (VL) of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VL SEQ ID Nos listed in Table 1. In some embodiments, the light chain variable region (VL) of the antibodies or antigen binding fragments disclosed herein comprises an amino acid sequence having about 90% identity to any one of the VL SEQ ID Nos listed in Table 1.

TABLE 1
Heavy chain variable region (VH), light chain variable region (VL), and
complementarity determining regions (CDRs) of anti-CSP-1 antibodies

| Antibody | SEQ ID NO: | | | | | | | |
|---|---|---|---|---|---|---|---|---|
| Number | VH | VL | HCDR1 | HCDR2 | HCDR3 | LCDR1 | LCDR2 | LCDR3 |
| 1 | 1 | 52 | 103 | 113 | 124 | 105 | 116 | 132 |
| 2 | 2 | 53 | 103 | 114 | 124 | 105 | 116 | 132 |
| 3 | 3 | 54 | 103 | 113 | 124 | 106 | 116 | 132 |
| 4 | 4 | 55 | 103 | 113 | 124 | 105 | 117 | 132 |
| 5 | 5 | 56 | 103 | 113 | 124 | 105 | 118 | 132 |
| 6 | 6 | 57 | 103 | 113 | 124 | 105 | 119 | 132 |
| 7 | 7 | 58 | 103 | 113 | 124 | 105 | 116 | 132 |
| 8 | 8 | 59 | 103 | 113 | 124 | 105 | 116 | 132 |
| 9 | 9 | 60 | 103 | 113 | 124 | 105 | 116 | 132 |
| 10 | 10 | 61 | 103 | 113 | 124 | 105 | 116 | 132 |
| 11 | 11 | 62 | 103 | 113 | 124 | 105 | 116 | 132 |
| 12 | 12 | 63 | 103 | 113 | 124 | 105 | 116 | 132 |
| 13 | 13 | 64 | 103 | 113 | 124 | 105 | 116 | 132 |
| 14 | 14 | 65 | 103 | 113 | 124 | 105 | 116 | 132 |
| 15 | 15 | 66 | 103 | 113 | 124 | 105 | 116 | 132 |
| 16 | 16 | 67 | 104 | 115 | 125 | 107 | 120 | 133 |
| 17 | 17 | 68 | 104 | 115 | 126 | 107 | 120 | 133 |
| 18 | 18 | 69 | 104 | 115 | 127 | 107 | 120 | 133 |
| 19 | 19 | 70 | 104 | 115 | 128 | 107 | 120 | 133 |
| 20 | 20 | 71 | 104 | 115 | 129 | 107 | 120 | 133 |
| 21 | 21 | 72 | 104 | 115 | 130 | 107 | 120 | 133 |
| 22 | 22 | 73 | 104 | 115 | 125 | 108 | 120 | 133 |
| 23 | 23 | 74 | 104 | 115 | 125 | 109 | 120 | 133 |
| 24 | 24 | 75 | 104 | 115 | 125 | 110 | 120 | 133 |
| 25 | 25 | 76 | 104 | 115 | 125 | 111 | 120 | 133 |
| 26 | 26 | 77 | 104 | 115 | 125 | 112 | 120 | 133 |
| 27 | 27 | 78 | 104 | 115 | 125 | 107 | 121 | 133 |
| 28 | 28 | 79 | 104 | 115 | 125 | 107 | 122 | 133 |
| 29 | 29 | 80 | 104 | 115 | 125 | 107 | 123 | 133 |
| 30 | 30 | 81 | 104 | 115 | 125 | 107 | 120 | 133 |
| 31 | 31 | 82 | 104 | 115 | 125 | 107 | 120 | 133 |
| 32 | 32 | 83 | 104 | 115 | 125 | 107 | 120 | 133 |
| 33 | 33 | 84 | 104 | 115 | 125 | 107 | 120 | 133 |
| 34 | 34 | 85 | 104 | 115 | 125 | 107 | 120 | 133 |
| 35 | 35 | 86 | 104 | 115 | 125 | 107 | 120 | 133 |
| 36 | 36 | 87 | 104 | 115 | 125 | 107 | 120 | 133 |
| 37 | 37 | 88 | 104 | 115 | 125 | 107 | 120 | 133 |
| 38 | 38 | 89 | 104 | 115 | 125 | 107 | 120 | 133 |
| 39 | 39 | 90 | 104 | 115 | 125 | 107 | 120 | 133 |
| 40 | 40 | 91 | 103 | 113 | 124 | 105 | 116 | 132 |
| 41 | 41 | 92 | 103 | 113 | 124 | 105 | 116 | 132 |
| 42 | 42 | 93 | 103 | 113 | 124 | 105 | 116 | 132 |
| 43 | 43 | 94 | 104 | 115 | 125 | 107 | 120 | 133 |
| 44 | 44 | 95 | 104 | 115 | 125 | 107 | 120 | 133 |
| 45 | 45 | 96 | 104 | 115 | 125 | 107 | 120 | 133 |
| 46 | 46 | 97 | 104 | 115 | 125 | 107 | 120 | 133 |
| 47 | 47 | 98 | 103 | 113 | 124 | 105 | 116 | 132 |
| 48 | 48 | 99 | 103 | 113 | 124 | 105 | 118 | 132 |
| 49 | 49 | 100 | 104 | 115 | 127 | 107 | 120 | 133 |
| 50 | 50 | 101 | 104 | 115 | 131 | 107 | 120 | 133 |
| 51 | 51 | 102 | 104 | 115 | 131 | 111 | 120 | 133 |

In some embodiments, the heavy chain variable region comprises a heavy chain complementary determining region 1 (HCDR1), a HCDR2, and a HCDR3. In some embodiments, the light chain variable region comprises a light chain complementary determining region 1 (LCDR1), a LCDR2, and a LCDR3. In some embodiments, the HCDR1 is a HCDR1 of a heavy chain variable region (VH) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VH SEQ ID Nos listed in Table 1. In some embodiments, the HCDR2 is a HCDR2 of a heavy chain variable region (VH) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VH SEQ ID Nos listed in Table 1. In some embodiments, the HCDR3 is a HCDR3 of a heavy chain variable region (VH) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VH SEQ ID Nos listed in Table 1.

In some embodiments, the LCDR1 is a LCDR1 of a light chain variable region (VL) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VL SEQ ID Nos listed in Table 1. In some embodiments, the LCDR2 is a LCDR2 of a light chain variable region (VL) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VL SEQ ID Nos listed in Table 1. In some embodiments, the LCDR3 is a LCDR3 of a light chain variable region (VL) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VL SEQ ID Nos listed in Table 1.

In some embodiments, an antibody or antigen-binding fragment thereof disclosed herein comprises an HCDR1, an HCDR2 and an HCDR3 of a heavy chain variable region (VH) comprising an amino acid sequence selected from SEQ ID Nos 1-51, as defined by Kabat.

In some embodiments, an antibody or antigen-binding fragment thereof disclosed herein comprises an LCDR1, an LCDR2 and an LCDR3 of a light chain variable region (VL) comprising an amino acid sequence selected from SEQ ID Nos 52-102, as defined by Kabat.

In some embodiments, an antibody or antigen-binding fragment thereof disclosed herein comprises an HCDR1, an HCDR2 and an HCDR3 of a heavy chain variable region (VH) comprising an amino acid sequence selected from SEQ ID Nos 1-51, as defined by Kabat, and an LCDR1, an LCDR2 and an LCDR3 of a light chain variable region (VL) comprising an amino acid sequence selected from SEQ ID Nos 52-102, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 1, as defined by Kabat, and an LCDR1, an LCDR2 and an LCDR3 of a light chain variable region (VL) comprising the amino acid sequence amino acid sequence of SEQ ID NO: 52, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 2, as defined by Kabat, and an LCDR1, an LCDR2 and an LCDR3 of a light chain variable region (VL) comprising the amino acid sequence amino acid sequence of SEQ ID NO: 53, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 3, as defined by Kabat, and an LCDR1, an LCDR2 and an LCDR3 of a light chain variable region (VL) comprising the amino acid sequence amino acid sequence of SEQ ID NO: 54, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 4, as defined by Kabat, and an LCDR1, an LCDR2 and an LCDR3 of a light chain variable region (VL) comprising the amino acid sequence amino acid sequence of SEQ ID NO: 55, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 5, as defined by Kabat, and an LCDR1, an LCDR2 and an LCDR3 of a light chain variable region (VL) comprising the amino acid sequence amino acid sequence of SEQ ID NO: 56, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 6, as defined by Kabat, and an LCDR1, an LCDR2 and an LCDR3 of a light chain variable region (VL) comprising the amino acid sequence amino acid sequence of SEQ ID NO: 57, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 7, as defined by Kabat, and an LCDR1, an LCDR2 and an LCDR3 of a light chain variable region (VL) comprising the amino acid sequence amino acid sequence of SEQ ID NO: 58, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 8, as defined by Kabat, and an LCDR1, an LCDR2 and an LCDR3 of a light chain variable region (VL) comprising the amino acid sequence amino acid sequence of SEQ ID NO: 59, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 9, as defined by Kabat, and an LCDR1, an LCDR2 and an LCDR3 of a light chain variable region (VL) comprising the amino acid sequence amino acid sequence of SEQ ID NO: 60, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 10, as defined by Kabat, and an LCDR1, an LCDR2 and an LCDR3 of a light chain variable region (VL) comprising the amino acid sequence amino acid sequence of SEQ ID NO: 61, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 11, as defined by Kabat, and an LCDR1, an LCDR2 and an LCDR3 of a light chain variable region (VL) comprising the amino acid sequence amino acid sequence of SEQ ID NO: 62, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 12, as defined by Kabat, and an LCDR1, an LCDR2 and an LCDR3 of a light chain variable region (VL) comprising the amino acid sequence amino acid sequence of SEQ ID NO: 63, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 13, as defined by Kabat, and an LCDR1, an LCDR2 and an LCDR3 of a light chain variable region (VL) comprising the amino acid sequence amino acid sequence of SEQ ID NO: 64, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 14, as defined by Kabat, and an LCDR1, an LCDR2 and an LCDR3 of a light chain variable region (VL) comprising the amino acid sequence amino acid sequence of SEQ ID NO: 65, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 15, as defined by Kabat, and an LCDR1, an LCDR2 and an LCDR3 of a light chain variable region (VL) comprising the amino acid sequence amino acid sequence of SEQ ID NO: 66, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 16, as defined by Kabat, and an LCDR1, an LCDR2 and an LCDR3 of a light chain variable region (VL) comprising the amino acid sequence amino acid sequence of SEQ ID NO: 67, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 17, as defined by Kabat, and an LCDR1, an LCDR2 and an LCDR3 of a light chain variable region (VL) comprising the amino acid sequence amino acid sequence of SEQ ID NO: 68, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 18, as defined by Kabat, and an LCDR1, an LCDR2 and an LCDR3 of a light chain variable region (VL) comprising the amino acid sequence amino acid sequence of SEQ ID NO: 69, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 19, as defined by Kabat, and an LCDR1, an LCDR2 and an LCDR3 of a light chain variable region (VL) comprising the amino acid sequence amino acid sequence of SEQ ID NO: 70, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 20, as defined by Kabat, and an LCDR1, an LCDR2 and an LCDR3 of a light chain variable region (VL) comprising the amino acid sequence amino acid sequence of SEQ ID NO: 71, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 21, as defined by Kabat, and an LCDR1, an LCDR2 and an LCDR3 of a light chain variable region (VL) comprising the amino acid sequence amino acid sequence of SEQ ID NO: 72, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 22, as defined by Kabat, and an LCDR1, an LCDR2 and an LCDR3 of a light chain variable region (VL) comprising the amino acid sequence amino acid sequence of SEQ ID NO: 73, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 23, as defined by Kabat, and an LCDR1, an LCDR2 and an LCDR3 of a light chain variable region (VL) comprising the amino acid sequence amino acid sequence of SEQ ID NO: 74, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 24, as defined by Kabat, and an LCDR1, an LCDR2 and an LCDR3 of a light chain variable region (VL) comprising the amino acid sequence amino acid sequence of SEQ ID NO: 75, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 25, as defined by Kabat, and an LCDR1, an LCDR2 and an LCDR3 of a light chain variable region (VL) comprising the amino acid sequence amino acid sequence of SEQ ID NO: 76, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 26, as defined by Kabat, and an LCDR1, an LCDR2 and an LCDR3 of a light chain variable region (VL) comprising the amino acid sequence amino acid sequence of SEQ ID NO: 77, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 27, as defined by Kabat, and an LCDR1, an LCDR2 and an LCDR3 of a light chain variable region (VL) comprising the amino acid sequence amino acid sequence of SEQ ID NO: 78, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 28, as defined by Kabat, and an LCDR1, an LCDR2 and an LCDR3 of a light chain variable region (VL) comprising the amino acid sequence amino acid sequence of SEQ ID NO: 79, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 29, as defined by Kabat, and an LCDR1, an LCDR2 and an LCDR3 of a light chain variable region (VL) comprising the amino acid sequence amino acid sequence of SEQ ID NO: 80, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 30, as defined by Kabat, and an LCDR1, an LCDR2 and an LCDR3 of a light chain variable region (VL) comprising the amino acid sequence amino acid sequence of SEQ ID NO: 81, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 31, as defined by Kabat, and an LCDR1, an LCDR2 and an LCDR3 of a light chain variable region (VL) comprising the amino acid sequence amino acid sequence of SEQ ID NO: 82, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 32, as defined by Kabat, and an LCDR1, an LCDR2 and an LCDR3 of a light chain variable region (VL) comprising the amino acid sequence amino acid sequence of SEQ ID NO: 83, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 33, as defined by Kabat, and an LCDR1, an LCDR2 and an LCDR3 of a light chain variable region (VL) comprising the amino acid sequence amino acid sequence of SEQ ID NO: 84, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 34, as defined by Kabat, and an LCDR1, an LCDR2 and an LCDR3 of a light chain variable region (VL) comprising the amino acid sequence amino acid sequence of SEQ ID NO: 85, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 35, as defined by Kabat, and an LCDR1, an LCDR2 and an LCDR3 of a light chain variable region (VL) comprising the amino acid sequence amino acid sequence of SEQ ID NO: 86, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 36, as defined by Kabat, and an LCDR1, an LCDR2 and an LCDR3 of a light chain variable region (VL) comprising the amino acid sequence amino acid sequence of SEQ ID NO: 87, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 37, as defined by Kabat, and an LCDR1, an LCDR2 and an LCDR3 of a light chain variable region (VL) comprising the amino acid sequence amino acid sequence of SEQ ID NO: 88, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 38, as defined by Kabat, and an LCDR1, an LCDR2 and an LCDR3 of a light chain variable region (VL) comprising the amino acid sequence amino acid sequence of SEQ ID NO: 89, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 39, as defined by Kabat, and an LCDR1, an LCDR2 and an LCDR3 of a light chain variable region (VL) comprising the amino acid sequence amino acid sequence of SEQ ID NO: 90, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 40, as defined by Kabat, and an LCDR1, an LCDR2 and an LCDR3 of a light chain variable region (VL) comprising the amino acid sequence amino acid sequence of SEQ ID NO: 91, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 41, as defined by Kabat, and an LCDR1, an LCDR2 and an LCDR3 of a light chain variable region (VL) comprising the amino acid sequence amino acid sequence of SEQ ID NO: 92, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 42, as defined by Kabat, and an LCDR1, an LCDR2 and an LCDR3 of a light chain variable region (VL) comprising the amino acid sequence amino acid sequence of SEQ ID NO: 93, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 43, as defined by Kabat, and an LCDR1, an LCDR2 and an LCDR3 of a light chain variable region (VL) comprising the amino acid sequence amino acid sequence of SEQ ID NO: 94, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 44, as defined by Kabat, and an LCDR1, an LCDR2 and an LCDR3 of a light chain variable region (VL) comprising the amino acid sequence amino acid sequence of SEQ ID NO: 95, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 45, as defined by Kabat, and an LCDR1, an LCDR2 and an LCDR3 of a light chain variable region (VL) comprising the amino acid sequence amino acid sequence of SEQ ID NO: 96, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 46, as defined by Kabat, and an LCDR1, an LCDR2 and an LCDR3 of a light chain variable region (VL) comprising the amino acid sequence amino acid sequence of SEQ ID NO: 97, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 47, as defined by Kabat, and an LCDR1, an LCDR2 and an LCDR3 of a light chain variable region (VL) comprising the amino acid sequence amino acid sequence of SEQ ID NO: 98, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 48, as defined by Kabat, and an LCDR1, an LCDR2 and an LCDR3 of a light chain variable region (VL) comprising the amino acid sequence amino acid sequence of SEQ ID NO: 99, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 49, as defined by Kabat, and an LCDR1, an LCDR2 and an LCDR3 of a light chain variable region (VL) comprising the amino acid sequence amino acid sequence of SEQ ID NO: 100, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 50, as defined by Kabat, and an LCDR1, an LCDR2 and an LCDR3 of a light chain variable region (VL) comprising the amino acid sequence amino acid sequence of SEQ ID NO: 101, as defined by Kabat.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 51, as defined by Kabat, and an LCDR1, an LCDR2 and an LCDR3 of a light chain variable region (VL) comprising the amino acid sequence amino acid sequence of SEQ ID NO: 102, as defined by Kabat.

In some embodiments, an antibody or antigen-binding fragment thereof disclosed herein comprises an HCDR1, an HCDR2 and an HCDR3 of a heavy chain variable region (VH) comprising an amino acid sequence selected from SEQ ID Nos 1-51, as defined by Chothia, and an LCDR1, an LCDR2 and an LCDR3 of a light chain variable region (VL) comprising an amino acid sequence selected from SEQ ID Nos 52-102, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 1, as defined by Chothia, and an LCDR1, an LCDR2 and an LCDR3 of a light chain variable region (VL) comprising the amino acid sequence amino acid sequence of SEQ ID NO: 52, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 2, as defined by Chothia, and an LCDR1, an LCDR2 and an LCDR3 of a light chain variable region (VL) comprising the amino acid sequence amino acid sequence of SEQ ID NO: 53, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 3, as defined by Chothia, and an LCDR1, an LCDR2 and an LCDR3 of a light chain variable region (VL) comprising the amino acid sequence amino acid sequence of SEQ ID NO: 54, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 4, as defined by Chothia, and an LCDR1, an LCDR2 and an LCDR3 of a light chain variable region (VL) comprising the amino acid sequence amino acid sequence of SEQ ID NO: 55, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 5, as defined by Chothia, and an LCDR1, an LCDR2 and an LCDR3 of a light chain variable region (VL) comprising the amino acid sequence amino acid sequence of SEQ ID NO: 56, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 6, as defined by Chothia, and an LCDR1, an LCDR2 and an LCDR3 of a light chain variable region (VL) comprising the amino acid sequence amino acid sequence of SEQ ID NO: 57, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 7, as defined by Chothia, and an LCDR1, an LCDR2 and an LCDR3 of a light chain variable region (VL) comprising the amino acid sequence amino acid sequence of SEQ ID NO: 58, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 8, as defined by Chothia, and an LCDR1, an LCDR2 and an LCDR3 of a light chain variable region (VL) comprising the amino acid sequence amino acid sequence of SEQ ID NO: 59, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 9, as defined by Chothia, and an LCDR1, an LCDR2 and an LCDR3 of a light chain variable region (VL) comprising the amino acid sequence amino acid sequence of SEQ ID NO: 60, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 10, as defined by Chothia, and an LCDR1, an LCDR2 and an LCDR3 of a light chain variable region (VL) comprising the amino acid sequence amino acid sequence of SEQ ID NO: 61, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 11, as defined by Chothia, and an LCDR1, an LCDR2 and an LCDR3 of a light chain variable region (VL) comprising the amino acid sequence amino acid sequence of SEQ ID NO: 62, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 12, as defined by Chothia, and an LCDR1, an LCDR2 and an LCDR3 of a light chain variable region (VL) comprising the amino acid sequence amino acid sequence of SEQ ID NO: 63, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 13, as defined by Chothia, and an LCDR1, an LCDR2 and an LCDR3 of a light chain variable region (VL) comprising the amino acid sequence amino acid sequence of SEQ ID NO: 64, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 14, as defined by Chothia, and an LCDR1, an LCDR2 and an LCDR3 of a light chain variable region (VL) comprising the amino acid sequence amino acid sequence of SEQ ID NO: 65, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 15, as defined by Chothia, and an LCDR1, an LCDR2 and an LCDR3 of a light chain variable region (VL) comprising the amino acid sequence amino acid sequence of SEQ ID NO: 66, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 16, as defined by Chothia, and an LCDR1, an LCDR2 and an LCDR3 of a light chain variable region (VL) comprising the amino acid sequence amino acid sequence of SEQ ID NO: 67, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 17, as defined by Chothia, and an LCDR1, an LCDR2 and an LCDR3 of a light chain variable region (VL) comprising the amino acid sequence amino acid sequence of SEQ ID NO: 68, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 18, as defined by Chothia, and an LCDR1, an LCDR2 and an LCDR3 of a light chain variable region (VL) comprising the amino acid sequence amino acid sequence of SEQ ID NO: 69, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 19, as defined by Chothia, and an LCDR1, an LCDR2 and an LCDR3 of a light chain variable region (VL) comprising the amino acid sequence amino acid sequence of SEQ ID NO: 70, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 20, as defined by Chothia, and an LCDR1, an LCDR2 and an LCDR3 of a light chain variable region (VL) comprising the amino acid sequence amino acid sequence of SEQ ID NO: 71, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 21, as defined by Chothia, and an LCDR1, an LCDR2 and an LCDR3 of a light chain variable region (VL) comprising the amino acid sequence amino acid sequence of SEQ ID NO: 72, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 22, as defined by Chothia, and an LCDR1, an LCDR2 and an LCDR3 of a light chain variable region (VL) comprising the amino acid sequence amino acid sequence of SEQ ID NO: 73, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 23, as defined by Chothia, and an LCDR1, an LCDR2 and an LCDR3 of a light chain variable region (VL) comprising the amino acid sequence amino acid sequence of SEQ ID NO: 74, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 24, as defined by Chothia, and an LCDR1, an LCDR2 and an LCDR3 of a light chain variable region (VL) comprising the amino acid sequence amino acid sequence of SEQ ID NO: 75, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 25, as defined by Chothia, and an LCDR1, an LCDR2 and an LCDR3 of a light chain variable region (VL) comprising the amino acid sequence amino acid sequence of SEQ ID NO: 76, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 26, as defined by Chothia, and an LCDR1, an LCDR2 and an LCDR3 of a light chain variable region (VL) comprising the amino acid sequence amino acid sequence of SEQ ID NO: 77, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 27, as defined by Chothia, and an LCDR1, an LCDR2 and an LCDR3 of a light chain variable region (VL) comprising the amino acid sequence amino acid sequence of SEQ ID NO: 78, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 28, as defined by Chothia, and an LCDR1, an LCDR2 and an LCDR3 of a light chain variable region (VL) comprising the amino acid sequence amino acid sequence of SEQ ID NO: 79, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 29, as defined by Chothia, and an LCDR1, an LCDR2 and an LCDR3 of a light chain variable region (VL) comprising the amino acid sequence amino acid sequence of SEQ ID NO: 80, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 30, as defined by Chothia, and an LCDR1, an LCDR2 and an LCDR3 of a light chain variable region (VL) comprising the amino acid sequence amino acid sequence of SEQ ID NO: 81, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 31, as defined by Chothia, and an LCDR1, an LCDR2 and an LCDR3 of a light chain variable region (VL) comprising the amino acid sequence amino acid sequence of SEQ ID NO: 82, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 32, as defined by Chothia, and an LCDR1, an LCDR2 and an LCDR3 of a light chain variable region (VL) comprising the amino acid sequence amino acid sequence of SEQ ID NO: 83, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 33, as defined by Chothia, and an LCDR1, an LCDR2 and an LCDR3 of a light chain variable region (VL) comprising the amino acid sequence amino acid sequence of SEQ ID NO: 84, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 34, as defined by Chothia, and an LCDR1, an LCDR2 and an LCDR3 of a light chain variable region (VL) comprising the amino acid sequence amino acid sequence of SEQ ID NO: 85, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 35, as defined by Chothia, and an LCDR1, an LCDR2 and an LCDR3 of a light chain variable region (VL) comprising the amino acid sequence amino acid sequence of SEQ ID NO: 86, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 36, as defined by Chothia, and an LCDR1, an LCDR2 and an LCDR3 of a light chain variable region (VL) comprising the amino acid sequence amino acid sequence of SEQ ID NO: 87, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 37, as defined by Chothia, and an LCDR1, an LCDR2 and an LCDR3 of a light chain variable region (VL) comprising the amino acid sequence amino acid sequence of SEQ ID NO: 88, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 38, as defined by Chothia, and an LCDR1, an LCDR2 and an LCDR3 of a light chain variable region (VL) comprising the amino acid sequence amino acid sequence of SEQ ID NO: 89, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 39, as defined by Chothia, and an LCDR1, an LCDR2 and an LCDR3 of a light chain variable region (VL) comprising the amino acid sequence amino acid sequence of SEQ ID NO: 90, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 40, as defined by Chothia, and an LCDR1, an LCDR2 and an LCDR3 of a light chain variable region (VL) comprising the amino acid sequence amino acid sequence of SEQ ID NO: 91, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 41, as defined by Chothia, and an LCDR1, an LCDR2 and an LCDR3 of a light chain variable region (VL) comprising the amino acid sequence amino acid sequence of SEQ ID NO: 92, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 42, as defined by Chothia, and an LCDR1, an LCDR2 and an LCDR3 of a light chain variable region (VL) comprising the amino acid sequence amino acid sequence of SEQ ID NO: 93, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 43, as defined by Chothia, and an LCDR1, an LCDR2 and an LCDR3 of a light chain variable region (VL) comprising the amino acid sequence amino acid sequence of SEQ ID NO: 94, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 44, as defined by Chothia, and an LCDR1, an LCDR2 and an LCDR3 of a light chain variable region (VL) comprising the amino acid sequence amino acid sequence of SEQ ID NO: 95, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 45, as defined by Chothia, and an LCDR1, an LCDR2 and an LCDR3 of a light chain variable region (VL) comprising the amino acid sequence amino acid sequence of SEQ ID NO: 96, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 46, as defined by Chothia, and an LCDR1, an LCDR2 and an LCDR3 of a light chain variable region (VL) comprising the amino acid sequence amino acid sequence of SEQ ID NO: 97, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 47, as defined by Chothia, and an LCDR1, an LCDR2 and an LCDR3 of a light chain variable region (VL) comprising the amino acid sequence amino acid sequence of SEQ ID NO: 98, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 48, as defined by Chothia, and an LCDR1, an LCDR2 and an LCDR3 of a light chain variable region (VL) comprising the amino acid sequence amino acid sequence of SEQ ID NO: 99, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 49, as defined by Chothia, and an LCDR1, an LCDR2 and an LCDR3 of a light chain variable region (VL) comprising the amino acid sequence amino acid sequence of SEQ ID NO: 100, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 50, as defined by Chothia, and an LCDR1, an LCDR2 and an LCDR3 of a light chain variable region (VL) comprising the amino acid sequence amino acid sequence of SEQ ID NO: 101, as defined by Chothia.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 51, as defined by Chothia, and an LCDR1, an LCDR2 and an LCDR3 of a light chain variable region (VL) comprising the amino acid sequence amino acid sequence of SEQ ID NO: 102, as defined by Chothia.

In some embodiments, an antibody or antigen-binding fragment thereof disclosed herein comprises an HCDR1, an HCDR2 and an HCDR3 of a heavy chain variable region (VH) comprising an amino acid sequence selected from SEQ ID Nos 1-51, as defined by AbM, and an LCDR1, an LCDR2 and an LCDR3 of a light chain variable region (VL) comprising an amino acid sequence selected from SEQ ID Nos 52-102, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 1, as defined by AbM, and an LCDR1, an LCDR2 and an LCDR3 of a light chain variable region (VL) comprising the amino acid sequence amino acid sequence of SEQ ID NO: 52, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 2, as defined by AbM, and an LCDR1, an LCDR2 and an LCDR3 of a light chain variable region (VL) comprising the amino acid sequence amino acid sequence of SEQ ID NO: 53, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 3, as defined by AbM, and an LCDR1, an LCDR2 and an LCDR3 of a light chain variable region (VL) comprising the amino acid sequence amino acid sequence of SEQ ID NO: 54, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 4, as defined by AbM, and an LCDR1, an LCDR2 and an LCDR3 of a light chain variable region (VL) comprising the amino acid sequence amino acid sequence of SEQ ID NO: 55, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 5, as defined by AbM, and an LCDR1, an LCDR2 and an LCDR3 of a light chain variable region (VL) comprising the amino acid sequence amino acid sequence of SEQ ID NO: 56, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 6, as defined by AbM, and an LCDR1, an LCDR2 and an LCDR3 of a light chain variable region (VL) comprising the amino acid sequence amino acid sequence of SEQ ID NO: 57, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 7, as defined by AbM, and an LCDR1, an LCDR2 and an LCDR3 of a light chain variable region (VL) comprising the amino acid sequence amino acid sequence of SEQ ID NO: 58, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 8, as defined by AbM, and an LCDR1, an LCDR2 and an LCDR3 of a light chain variable region (VL) comprising the amino acid sequence amino acid sequence of SEQ ID NO: 59, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 9, as defined by AbM, and an LCDR1, an LCDR2 and an LCDR3 of a light chain variable region (VL) comprising the amino acid sequence amino acid sequence of SEQ ID NO: 60, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 10, as defined by AbM, and an LCDR1, an LCDR2 and an LCDR3 of a light chain variable region (VL) comprising the amino acid sequence amino acid sequence of SEQ ID NO: 61, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 11, as defined by AbM, and an LCDR1, an LCDR2 and an LCDR3 of a light chain variable region (VL) comprising the amino acid sequence amino acid sequence of SEQ ID NO: 62, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 12, as defined by AbM, and an LCDR1, an LCDR2 and an LCDR3 of a light chain variable region (VL) comprising the amino acid sequence amino acid sequence of SEQ ID NO: 63, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 13, as defined by AbM, and an LCDR1, an LCDR2 and an LCDR3 of a light chain variable region (VL) comprising the amino acid sequence amino acid sequence of SEQ ID NO: 64, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 14, as defined by AbM, and an LCDR1, an LCDR2 and an LCDR3 of a light chain variable region (VL) comprising the amino acid sequence amino acid sequence of SEQ ID NO: 65, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 15, as defined by AbM, and an LCDR1, an LCDR2 and an LCDR3 of a light chain variable region (VL) comprising the amino acid sequence amino acid sequence of SEQ ID NO: 66, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 16, as defined by AbM, and an LCDR1, an LCDR2 and an LCDR3 of a light chain variable region (VL) comprising the amino acid sequence amino acid sequence of SEQ ID NO: 67, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 17, as defined by AbM, and an LCDR1, an LCDR2 and an LCDR3 of a light chain variable region (VL) comprising the amino acid sequence amino acid sequence of SEQ ID NO: 68, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 18, as defined by AbM, and an LCDR1, an LCDR2 and an LCDR3 of a light chain variable region (VL) comprising the amino acid sequence amino acid sequence of SEQ ID NO: 69, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 19, as defined by AbM, and an LCDR1, an LCDR2 and an LCDR3 of a light chain variable region (VL) comprising the amino acid sequence amino acid sequence of SEQ ID NO: 70, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 20, as defined by AbM, and an LCDR1, an LCDR2 and an LCDR3 of a light chain variable region (VL) comprising the amino acid sequence amino acid sequence of SEQ ID NO: 71, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 21, as defined by AbM, and an LCDR1, an LCDR2 and an LCDR3 of a light chain variable region (VL) comprising the amino acid sequence amino acid sequence of SEQ ID NO: 72, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 22, as defined by AbM, and an LCDR1, an LCDR2 and an LCDR3 of a light chain variable region (VL) comprising the amino acid sequence amino acid sequence of SEQ ID NO: 73, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 23, as defined by AbM, and an LCDR1, an LCDR2 and an LCDR3 of a light chain variable region (VL) comprising the amino acid sequence amino acid sequence of SEQ ID NO: 74, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 24, as defined by AbM, and an LCDR1, an LCDR2 and an LCDR3 of a light chain variable region (VL) comprising the amino acid sequence amino acid sequence of SEQ ID NO: 75, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 25, as defined by AbM, and an LCDR1, an LCDR2 and an LCDR3 of a light chain variable region (VL) comprising the amino acid sequence amino acid sequence of SEQ ID NO: 76, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 26, as defined by AbM, and an LCDR1, an LCDR2 and an LCDR3 of a light chain variable region (VL) comprising the amino acid sequence amino acid sequence of SEQ ID NO: 77, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 27, as defined by AbM, and an LCDR1, an LCDR2 and an LCDR3 of a light chain variable region (VL) comprising the amino acid sequence amino acid sequence of SEQ ID NO: 78, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 28, as defined by AbM, and an LCDR1, an LCDR2 and an LCDR3 of a light chain variable region (VL) comprising the amino acid sequence amino acid sequence of SEQ ID NO: 79, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 29, as defined by AbM, and an LCDR1, an LCDR2 and an LCDR3 of a light chain variable region (VL) comprising the amino acid sequence amino acid sequence of SEQ ID NO: 80, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 30, as defined by AbM, and an LCDR1, an LCDR2 and an LCDR3 of a light chain variable region (VL) comprising the amino acid sequence amino acid sequence of SEQ ID NO: 81, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 31, as defined by AbM, and an LCDR1, an LCDR2 and an LCDR3 of a light chain variable region (VL) comprising the amino acid sequence amino acid sequence of SEQ ID NO: 82, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 32, as defined by AbM, and an LCDR1, an LCDR2 and an LCDR3 of a light chain variable region (VL) comprising the amino acid sequence amino acid sequence of SEQ ID NO: 83, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 33, as defined by AbM, and an LCDR1, an LCDR2 and an LCDR3 of a light chain variable region (VL) comprising the amino acid sequence amino acid sequence of SEQ ID NO: 84, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 34, as defined by AbM, and an LCDR1, an LCDR2 and an LCDR3 of a light chain variable region (VL) comprising the amino acid sequence amino acid sequence of SEQ ID NO: 85, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 35, as defined by AbM, and an LCDR1, an LCDR2 and an LCDR3 of a light chain variable region (VL) comprising the amino acid sequence amino acid sequence of SEQ ID NO: 86, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 36, as defined by AbM, and an LCDR1, an LCDR2 and an LCDR3 of a light chain variable region (VL) comprising the amino acid sequence amino acid sequence of SEQ ID NO: 87, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 37, as defined by AbM, and an LCDR1, an LCDR2 and an LCDR3 of a light chain variable region (VL) comprising the amino acid sequence amino acid sequence of SEQ ID NO: 88, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 38, as defined by AbM, and an LCDR1, an LCDR2 and an LCDR3 of a light chain variable region (VL) comprising the amino acid sequence amino acid sequence of SEQ ID NO: 89, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 39, as defined by AbM, and an LCDR1, an LCDR2 and an LCDR3 of a light chain variable region (VL) comprising the amino acid sequence amino acid sequence of SEQ ID NO: 90, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 40, as defined by AbM, and an LCDR1, an LCDR2 and an LCDR3 of a light chain variable region (VL) comprising the amino acid sequence amino acid sequence of SEQ ID NO: 91, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 41, as defined by AbM, and an LCDR1, an LCDR2 and an LCDR3 of a light chain variable region (VL) comprising the amino acid sequence amino acid sequence of SEQ ID NO: 92, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 42, as defined by AbM, and an LCDR1, an LCDR2 and an LCDR3 of a light chain variable region (VL) comprising the amino acid sequence amino acid sequence of SEQ ID NO: 93, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 43, as defined by AbM, and an LCDR1, an LCDR2 and an LCDR3 of a light chain variable region (VL) comprising the amino acid sequence amino acid sequence of SEQ ID NO: 94, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 44, as defined by AbM, and an LCDR1, an LCDR2 and an LCDR3 of a light chain variable region (VL) comprising the amino acid sequence amino acid sequence of SEQ ID NO: 95, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 45, as defined by AbM, and an LCDR1, an LCDR2 and an LCDR3 of a light chain variable region (VL) comprising the amino acid sequence amino acid sequence of SEQ ID NO: 96, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 46, as defined by AbM, and an LCDR1, an LCDR2 and an LCDR3 of a light chain variable region (VL) comprising the amino acid sequence amino acid sequence of SEQ ID NO: 97, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 47, as defined by AbM, and an LCDR1, an LCDR2 and an LCDR3 of a light chain variable region (VL) comprising the amino acid sequence amino acid sequence of SEQ ID NO: 98, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 48, as defined by AbM, and an LCDR1, an LCDR2 and an LCDR3 of a light chain variable region (VL) comprising the amino acid sequence amino acid sequence of SEQ ID NO: 99, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 49, as defined by AbM, and an LCDR1, an LCDR2 and an LCDR3 of a light chain variable region (VL) comprising the amino acid sequence amino acid sequence of SEQ ID NO: 100, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 50, as defined by AbM, and an LCDR1, an LCDR2 and an LCDR3 of a light chain variable region (VL) comprising the amino acid sequence amino acid sequence of SEQ ID NO: 101, as defined by AbM.

In some embodiments, an antibody or antigen-binding fragment thereof disclosed herein comprises an HCDR1, an HCDR2 and an HCDR3 of a heavy chain variable region (VH) comprising the amino acid sequence of SEQ ID NO: 51, as defined by AbM, and an LCDR1, an LCDR2 and an LCDR3 of a light chain variable region (VL) comprising the amino acid sequence amino acid sequence of SEQ ID NO: 102, as defined by AbM.

In some embodiments, the HCDR1 of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the HCDR1 SEQ ID Nos listed in Table 1. In some embodiments, HCDR1 comprises an amino acid sequence having at least about 90% identity to any one of the HCDR1 SEQ ID Nos listed in Table 1.

In some embodiments, the HCDR2 of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the HCDR2 SEQ ID Nos listed in Table 1. In some embodiments, HCDR2 comprises an amino acid sequence having at least about 90% identity to any one of the HCDR2 SEQ ID Nos listed in Table 1.

In some embodiments, the HCDR3 of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the HCDR3 SEQ ID Nos listed in Table 1. In some embodiments, HCDR3 comprises an amino acid sequence having at least about 90% identity to any one of the HCDR3 SEQ ID Nos listed in Table 1.

In some embodiments, the LCDR1 of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the LCDR1 SEQ ID Nos listed in Table 1. In some embodiments, LCDR1 comprises an amino acid sequence having at least about 90% identity to any one of the LCDR1 SEQ ID Nos listed in Table 1.

In some embodiments, the LCDR2 of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the LCDR2 SEQ ID Nos listed in Table 1. In some embodiments, LCDR2 comprises an amino acid sequence having at least about 90% identity to any one of the LCDR2 SEQ ID Nos listed in Table 1.

In some embodiments, the LCDR3 of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the LCDR3 SEQ ID Nos listed in Table 1. In some embodiments, LCDR3 comprises an amino acid sequence having at least about 90% identity to any one of the LCDR3 SEQ ID Nos listed in Table 1.

The disclosure provides antibodies that were developed to target CSP-1. In some embodiments, the heavy chain variable region (VH) of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VH sequences listed in Table 2. In some embodiments, the heavy chain variable region of the antibodies or antigen binding fragments disclosed herein comprises an amino acid sequence having at least about 90% identity to any one of the VH sequences listed in Table 2. In some embodiments, the light chain variable region (VL) of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VL sequences listed in Table 2. In some embodiments, the light chain variable region (VL) of the antibodies or antigen binding fragments disclosed herein comprises an amino acid sequence having about 90% identity to any one of the VL sequences listed in Table 2.

In some embodiments, the HCDR1 is a HCDR1 of a heavy chain variable region (VH) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VH SEQ ID Nos listed in Table 2. In some embodiments, the HCDR2 is a HCDR2 of a heavy chain variable region (VH) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VH SEQ ID Nos listed in Table 2. In some embodiments, the HCDR3 is a HCDR3 of a heavy chain variable region (VH) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VH SEQ ID Nos listed in Table 2.

In some embodiments, the LCDR1 is a LCDR1 of a light chain variable region (VL) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VL SEQ ID Nos listed in Table 2. In some embodiments, the LCDR2 is a LCDR2 of a light chain variable region (VL) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VL SEQ ID Nos listed in Table 2. In some embodiments, the LCDR3 is a LCDR3 of a light chain variable region (VL) comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of the VL SEQ ID Nos listed in Table 2.

TABLE 2
lists the VH an VL sequences of other antibodies that were developed to target CSP-1.

| Antibody | | |
|---|---|---|
| No: | VH sequence | VL sequence |
| 52 | QIQLVQSGPELKKPGETVKISCKASGYTFTDYSM | DIVMTQAAPSVPVTPGESVSISCRSSKSLLH |
| | HWVKQAPGKGLKWMGWISTETGEPTYADDFKG | SNGNTYLYWFLQRPGQSPQLLIYRMSNLAS |
| | RFAFSLETSASTAYLQINNLKNEDTATYFCAPGGF | GVPDRFSGSGSGTAFTLRISRVEAEDVGVY |
| | AYWGQGTLVTVSA (SEQ ID NO: 195) | YCMQHLEYPLTFGAGTKLELK (SEQ ID NO: |
| | | 295) |
| 53 | QIQLVQSGPELKKPGETVKISCKASGYTFTDYSM | DIVMTQAAPSVPVTPGESVSISCRSSKSLLH |
| | HWVKQAPGKGLKWMGWIVTETGEPTYADDFKG | SNGNTYLYWFLQRPGQSPQLLIYRMSNLAS |
| | RFAFSLETSASTAYLQINNLKNEDTATYFCAPGGF | GVPDRFSGSGSGTAFTLRISRVEAEDVGVY |
| | AYWGQGTLVTVSA (SEQ ID NO: 196) | YCMQHLEYPLTFGAGTKLELK (SEQ ID NO: |
| | | 296) |
| 54 | QIQLVQSGPELKKPGETVKISCKASGYTFTDYSM | DIVMTQAAPSVPVTPGESVSISCRSSKSLLH |
| | HWVKQAPGKGLKWMGWIDTETGEPTYADDFKG | SNGNTYLYWFLQRPGQSPQLLIYRMSNLAS |
| | RFAFSLETSASTAYLQINNLKNEDTATYFCAPGGF | GVPDRFSGSGSGTAFTLRISRVEAEDVGVY |
| | AYWGQGTLVTVSA (SEQ ID NO: 197) | YCMQHLEYPLTFGAGTKLELK (SEQ ID NO: |
| | | 297) |
| 55 | QIQLVQSGPELKKPGETVKISCKASGYTFTDYSM | DIVMTQAAPSVPVTPGESVSISCRSSKSLLH |
| | HWVKQAPGKGLKWMGWIETETGEPTYADDFKG | SNGNTYLYWFLQRPGQSPQLLIYRMSNLAS |
| | RFAFSLETSASTAYLQINNLKNEDTATYFCAPGGF | GVPDRFSGSGSGTAFTLRISRVEAEDVGVY |
| | AYWGQGTLVTVSA (SEQ ID NO: 198) | YCMQHLEYPLTFGAGTKLELK (SEQ ID NO: |
| | | 298) |
| 56 | QIQLVQSGPELKKPGETVKISCKASGYTFTDYSM | DIVMTQAAPSVPVTPGESVSISCRSSKSLLH |
| | HWVKQAPGKGLKWMGWINTETGEPTYADDFKG | SNGNTYLYWFLQRPGQSPQLLIYRMSNLAS |
| | RFAFSLETSASTAYLQINNLKNEDTATYFCARGG | GVPDRFSGSGSGTAFTLRISRVEAEDVGVY |
| | FAYWGQGTLVTVSA (SEQ ID NO: 199) | YCMQHLEYPLTFGAGTKLELK (SEQ ID NO: |
| | | 299) |
| 57 | QIQLVQSGPELKKPGETVKISCKASGYTFTDYSM | DIVMTQAAPSVPVTPGESVSISCRSSKSLLH |
| | HWVKQAPGKGLKWMGWINTETGEPTYADDFKG | SVGNTYLYWFLQRPGQSPQLLIYRMSNLAS |
| | RFAFSLETSASTAYLQINNLKNEDTATYFCAPGGF | GVPDRFSGSGSGTAFTLRISRVEAEDVGVY |
| | AYWGQGTLVTVSA (SEQ ID NO: 200) | YCMQHLEYPLTFGAGTKLELK (SEQ ID NO: |
| | | 300) |
| 58 | QIQLVQSGPELKKPGETVKISCKASGYTFTDYSM | DIVMTQAAPSVPVTPGESVSISCRSSKSLLH |
| | HWVKQAPGKGLKWMGWINTETGEPTYADDFKG | SDGNTYLYWFLQRPGQSPQLLIYRMSNLAS |
| | RFAFSLETSASTAYLQINNLKNEDTATYFCAPGGF | GVPDRFSGSGSGTAFTLRISRVEAEDVGVY |
| | AYWGQGTLVTVSA (SEQ ID NO: 201) | YCMQHLEYPLTFGAGTKLELK (SEQ ID NO: |
| | | 301) |
| 59 | QIQLVQSGPELKKPGETVKISCKASGYTFTDYSM | DIVMTQAAPSVPVTPGESVSISCRSSKSLLH |
| | HWVKQAPGKGLKWMGWINTETGEPTYADDFKG | SQGNTYLYWFLQRPGQSPQLLIYRMSNLAS |
| | RFAFSLETSASTAYLQINNLKNEDTATYFCAPGGF | GVPDRFSGSGSGTAFTLRISRVEAEDVGVY |
| | AYWGQGTLVTVSA (SEQ ID NO: 202) | YCMQHLEYPLTFGAGTKLELK (SEQ ID NO: |
| | | 302) |
| 60 | QIQLVQSGPELKKPGETVKISCKASGYTFTDYSM | DIVMTQAAPSVPVTPGESVSISCRSSKSLLH |
| | HWVKQAPGKGLKWMGWINTETGEPTYADDFKG | SEGNTYLYWFLQRPGQSPQLLIYRMSNLAS |
| | RFAFSLETSASTAYLQINNLKNEDTATYFCAPGGF | GVPDRFSGSGSGTAFTLRISRVEAEDVGVY |
| | AYWGQGTLVTVSA (SEQ ID NO: 203) | YCMQHLEYPLTFGAGTKLELK (SEQ ID NO: |
| | | 303) |
| 61 | QIQLVQSGPGLKQPGGSVRISCAASGYTFTDYSM | DIVMTQAAPSVPVTPGESVSISCRSSKSLLH |
| | HWVKQAPGKGLKWMGWINTETGEPTYADDFKG | SNGNTYLYWFLQRPGQSPQLLIYRMSNLAS |
| | RFTFSLDTSKSTAYLQINSLRAEDTATYFCAPGGF | GVPDRFSGSGSGTAFTLRISRVEAEDVGVY |
| | AYWGQGTLVTVSS (SEQ ID NO: 204) | YCMQHLEYPLTFGAGTKLELK (SEQ ID NO: |
| | | 304) |
| 62 | EIQLVQSGPGLVQPGGSVRISCAASGYTFTDYSM | DIVMTQAAPSVPVTPGESVSISCRSSKSLLH |
| | HWVRQAPGKGLEWMGWINTETGEPTYADSFKG | SNGNTYLYWFLQRPGQSPQLLIYRMSNLAS |
| | RFTFSLDNSKNTAYLQINSLRAEDTAVYFCAPGG | GVPDRFSGSGSGTAFTLRISRVEAEDVGVY |
| | FAYWGQGTLVTVSS (SEQ ID NO: 205) | YCMQHLEYPLTFGAGTKLELK (SEQ ID NO: |
| | | 305) |
| 63 | QIQLVQSGPEVKKPGASVKISCKASGYTFTDYSM | DIVMTQSAPSLPVTPGQSVSISCRSSKSLLHS |
| | HWVRQAPGQGLEWMGWINTETGEPTYAQKFQG | NGNTYLYWFLQKPGQSPQLLIYRMSNRAS |
| | RFTFTLDTSTSTAYLEISSLRSEDTAVYFCAPGGF | GVPDRFSGSGSGTDFTLKISRVEAEDVGVY |
| | AYWGQGTLVTVSS (SEQ ID NO: 206) | YCMQHLEYPLTFGQGTKLEIK (SEQ ID NO: |
| | | 306) |
| 64 | EIQLVQSGPGLVQPGGSVRISCAASGYTFTDYSM | DIVMTQSAPSVPVTPGQSVSISCRSSKSLLH |
| | HWVRQAPGKGLEWMGWINTETGEPTYADSFKG | SNGNTYLYWFLQKPGQSPQLLIYRMSNRAS |
| | RFTFSLDNSKNTAYLQINSLRAEDTAVYFCAPGG | GVPDRFSGSGSGTDFTLKISRVEAEDVGVY |
| | FAYWGQGTLVTVSS (SEQ ID NO: 207) | YCMQHLEYPLTFGQGTKLEIK (SEQ ID NO: |
| | | 307) |
| 65 | EIQLVQSGPGLVQPGGSVRISCAASGYTFTDYSM | DIVMTQSAPSLPVTPGQSVSISCRSSKSLLHS |
| | HWVRQAPGKGLEWMGWINTETGEPTYADSFKG | NGNTYLYWFLQKPGQSPQLLIYRMSNRAS |
| | RFTFSLDNSKNTAYLQINSLRAEDTAVYFCAPGG | GVPDRFSGSGSGTDFTLKISRVEAEDVGVY |
| | FAYWGQGTLVTVSS (SEQ ID NO: 208) | YCMQHLEYPLTFGQGTKLEIK (SEQ ID NO: |
| | | 308) |
| 66 | EVQLVESGGDLVKPGGSLKLSCAASGFTFSRYGM | DIVMTQAAFSNPVTLGTSASISCRSSKSLLH |
| | SWVRQTPDKRLEWVATISIGGTYTYYPDSMKGR | SNGITYLYWYLQKPGQSPQLLIYQMSNLAS |
| | FTISRDNAKNTLYLQMSSLKSEDTAMYYCARRG | GVPDRFSSSGSGTDFTLRISRVEAEDVGVY |
| | YGVYSYYGMDYWGQGTSVTVSS (SEQ ID NO: | YCAQNLELPLTFGAGTKLELK (SEQ ID NO: |
| | 209) | 309) |
| 67 | EVQLVESGGDLVKPGGSLKLSCAASGFTFSRYGM | DIVMTQAAFSNPVTLGTSASISCRSSKSLLH |
| | SWVRQTPDKRLEWVATISIGGTYTYYPDSMKGR | SNGITYLYWYLQKPGQSPQLLIYQMSNLAS |
| | FTISRDNAKNTLYLQMSSLKSEDTAMYYCARRG | GVPDRFSSSGSGTDFTLRISRVEAEDVGVY |
| | YGDYSYYGMDYWGQGTSVTVSS (SEQ ID NO: | YCAQNLELPLTFGAGTKLELK (SEQ ID NO: |
| | 210) | 310) |
| 68 | EVQLVESGGDLVKPGGSLKLSCAASGFTFSRYGM | DIVMTQAAFSNPVTLGTSASISCRSSKSLLH |
| | SWVRQTPDKRLEWVATISIGGTYTYYPDSMKGR | SNGITYLYWYLQKPGQSPQLLIYQMSNLAS |
| | FTISRDNAKNTLYLQMSSLKSEDTAMYYCARRG | GVPDRFSSSGSGTDFTLRISRVEAEDVGVY |
| | YGEYSYYGMDYWGQGTSVTVSS (SEQ ID NO: | YCAQNLELPLTFGAGTKLELK (SEQ ID NO: |
| | 211) | 311) |
| 69 | EVQLVESGGEVVKPGASLKLSCKASGFTFSRYGM | DIVMTQSALSLPVTLGQSASISCRSSKSLLH |
| | SWVRQAPGQRLEWVATISIGGTYTYYPDSMKGR | SNGITYLYWYLQKPGQSPQLLIYQMSNRAS |
| | FTITRDNSTNTLYLEMSSLRSEDTAMYYCARRGY | GVPDRFSSSGSGTDFTLKISRVEAEDVGVY |
| | GNYSYYGMDYWGQGTLVTVSS (SEQ ID NO: | YCAQNLELPLTFGQGTKLEIK (SEQ ID NO: |
| | 212) | 312) |
| 70 | QVQLVQSGSELKKPGASVKVSCKASGYTFTDYS | DIVMTQSPLSLPVTPGEPASISCRSSKSLLHS |
| | MNWVRQAPGQGLEWMGWINTETGEPTYAQGFT | NGNTYLDWYLQKPGQSPQLLIYRMSNRAS |
| | GRFVFSLDTSVSTAYLQISSLKAEDTAVYYCAPG | GVPDRFSGSGSGTDFTLKISRVEAEDVGVY |
| | GFAYWGQGTLVTVSS (SEQ ID NO: 213) | YCMQHLEYPLTFGQGTKLEIK (SEQ ID NO: |
| | | 313) |
| 71 | QVQLVQSGSELKKPGASVKVSCKASGYTFTDYS | DIVMTQSPLSLPVTPGEPASISCRSSKSLLHS |
| | MNWVRQAPGQGLEWMGWINTETGEPTYAQGFT | NGNTYLYWFLQKPGQSPQLLIYRMSNLAS |
| | GRFVFSLDTSVSTAYLQISSLKAEDTAVYYCAPG | GVPDRFSGSGSGTDFTLKISRVEAEDVGVY |
| | GFAYWGQGTLVTVSS (SEQ ID NO: 214) | YCMQHLEYPLTFGAGTKLELK (SEQ ID NO: |
| | | 314) |
| 72 | QIQLVQSGSELKKPGASVKVSCKASGYTFTDYSM | DIVMTQSPLSLPVTPGEPASISCRSSKSLLHS |
| | HWVRQAPGQGLEWMGWINTETGEPTYAQGFTG | NGNTYLDWYLQKPGQSPQLLIYRMSNRAS |
| | RFVFSLDTSVSTAYLQISSLKAEDTAVYFCAPGGF | GVPDRFSGSGSGTDFTLKISRVEAEDVGVY |
| | AYWGQGTLVTVSS (SEQ ID NO: 215) | YCMQHLEYPLTFGQGTKLEIK (SEQ ID NO: |
| | | 315) |
| 73 | EVQLVESGGGLVKPGGSLRLSCAASGFTFSRYGM | DIVMTQSPLSLPVTPGEPASISCRSSKSLLHS |
| | NWVRQAPGKGLEWVSSISIGGTYTYYADSVKGR | NGITYLDWYLQKPGQSPQLLIYQMSNRASG |
| | FTISRDNAKNSLYLQMNSLRAEDTAVYYCARRG | VPDRFSGSGSGTDFTLKISRVEAEDVGVYY |
| | YGNYSYYGMDYWGQGTLVTVSS (SEQ ID NO: | CAQNLELPLTFGQGTKLEIK (SEQ ID NO: |
| | 216) | 316) |
| 74 | EVQLVESGGGLVKPGGSLRLSCAASGFTFSRYGM | DIVMTQSPLSLPVTPGEPASISCRSSKSLLHS |
| | SWVRQAPGKGLEWVSSISIGGTYTYYADSVKGRF | NGITYLDWYLQKPGQSPQLLIYQMSNRASG |
| | TISRDNAKNSLYLQMNSLRAEDTAVYYCARRGY | VPDRFSGSGSGTDFTLKISRVEAEDVGVYY |
| | GNYSYYGMDYWGQGTLVTVSS (SEQ ID NO: | CAQNLELPLTFGQGTKLEIK (SEQ ID NO: |
| | 217) | 317) |
| 75 | EVQLVESGGGLVKPGGSLRLSCAASGFTFSRYGM | DIVMTQSPLSLPVTPGEPASISCRSSKSLLHS |
| | SWVRQAPGKGLEWVSSISIGGTYTYYADSVKGRF | NGITYLYWYLQKPGQSPQLLIYQMSNRASG |
| | TISRDNAKNSLYLQMNSLRAEDTAVYYCARRGY | VPDRFSGSGSGTDFTLKISRVEAEDVGVYY |
| | GNYSYYGMDYWGQGTLVTVSS (SEQ ID NO: | CAQNLELPLTFGQGTKLEIK (SEQ ID NO: |
| | 218) | 318) |
| 76 | EVQLVESGGGLVKPGGSLRLSCAASGFTFSRYGM | DIVMTQSPLSLPVTPGEPASISCRSSKSLLHS |
| | SWVRQAPGKGLEWVSSISIGGTYTYYADSVKGRF | NGITYLYWYLQKPGQSPQLLIYQMSNLASG |
| | TISRDNAKNSLYLQMNSLRAEDTAVYYCARRGY | VPDRFSGSGSGTDFTLKISRVEAEDVGVYY |
| | GNYSYYGMDYWGQGTLVTVSS (SEQ ID NO: | CAQNLELPLTFGQGTKLEIK (SEQ ID NO: |
| | 219) | 319) |
| 77 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTNYADDF | NGYTYLYWYLQKPGQSPQLLIYRVSNLAS |
| | KGRVTMTRDTSISTAYMELSRLRSDDTATYFCAP | GVPDRFSGSGSGSDFTLKISWVEAEDVGVY |
| | GGFAYWGQGTLVTVSS (SEQ ID NO: 220) | YCMQATQFPLTFGQGTKLEIK (SEQ ID NO: |
| | | 320) |
| 78 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTNYADDF | NGYTYLYWYLQKPGQSPQLLIYRVSNHLS |
| | KGRVTMTRDTSISTAYMELSRLRSDDTATYFCAP | GVPDRFSGSGSGSDFTLKISWVEAEDVGVY |
| | GGFAYWGQGTLVTVSS (SEQ ID NO: 221) | YCMQATQFPLTFGAGTKLEIK (SEQ ID NO: |
| | | 321) |
| 79 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTNYADDF | NGYTYLYWYLQKPGQSPQLLIYRVSNLLSG |
| | KGRVTMTRDTSISTAYMELSRLRSDDTATYFCAP | VPDRFSGSGSGSDFTLKISWVEAEDVGVYY |
| | GGFAYWGQGTLVTVSS (SEQ ID NO: 222) | CMQATQFPLTFGQGTKLEIK (SEQ ID NO: |
| | | 322) |
| 80 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTNYADDF | NGYTYLYWFLQKPGQSPQLLIYRVSNLASG |
| | KGRVTMTRDTSISTAYMELSRLRSDDTATYFCAP | VPDRFSGSGSGSDFTLKISWVEAEDVGVYY |
| | GGFAYWGQGTLVTVSS (SEQ ID NO: 223) | CMQATQFPLTFGAGTKLEIK (SEQ ID NO: |
| | | 323) |
| 81 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTNYADDF | NGYTYLYWYLQKPGQSPQLLIYRVSNLAS |
| | QGRVTMTRDTSISTAYMELSRLRSDDTATYFCAP | GVPDRFSGSGSGSDFTLKISWVEAEDVGVY |
| | GGFAYWGQGTLVTVSS (SEQ ID NO: 224) | YCMQATQFPLTFGQGTKLEIK (SEQ ID NO: |
| | | 324) |
| 82 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTNYADDF | NGYTYLYWYLQKPGQSPQLLIYRVSNHLS |
| | QGRVTMTRDTSISTAYMELSRLRSDDTATYFCAP | GVPDRFSGSGSGSDFTLKISWVEAEDVGVY |
| | GGFAYWGQGTLVTVSS (SEQ ID NO: 225) | YCMQATQFPLTFGAGTKLEIK (SEQ ID NO: |
| | | 325) |
| 83 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTNYADDF | NGYTYLYWYLQKPGQSPQLLIYRVSNLLSG |
| | QGRVTMTRDTSISTAYMELSRLRSDDTATYFCAP | VPDRFSGSGSGSDFTLKISWVEAEDVGVYY |
| | GGFAYWGQGTLVTVSS (SEQ ID NO: 226) | CMQATQFPLTFGQGTKLEIK (SEQ ID NO: |
| | | 326) |
| 84 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTNYADDF | NGYTYLYWFLQKPGQSPQLLIYRVSNLASG |
| | QGRVTMTRDTSISTAYMELSRLRSDDTATYFCAP | VPDRFSGSGSGSDFTLKISWVEAEDVGVYY |
| | GGFAYWGQGTLVTVSS (SEQ ID NO: 227) | CMQATQFPLTFGAGTKLEIK (SEQ ID NO: |
| | | 327) |
| 85 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTNYADDF | NGYTYLYWYLQKPGQSPQLLIYRVSNLAS |
| | KGRVTMTRDTSISTAYMELSRLRSDDTATYFCAP | GVPDRFSGSGSGSDFTLKISWVEAEDVGVY |
| | GGFAYWGQGTLVTVSS (SEQ ID NO: 228) | YCMQATQFPLTFGQGTKLEIK (SEQ ID NO: |
| | | 328) |
| 86 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTNYADDF | NGYTYLYWYLQKPGQSPQLLIYRVSNHLS |
| | KGRVTMTRDTSISTAYMELSRLRSDDTATYFCAP | GVPDRFSGSGSGSDFTLKISWVEAEDVGVY |
| | GGFAYWGQGTLVTVSS (SEQ ID NO: 229) | YCMQATQFPLTFGAGTKLEIK (SEQ ID NO: |
| | | 329) |
| 87 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTNYADDF | NGYTYLYWYLQKPGQSPQLLIYRVSNLLSG |
| | KGRVTMTRDTSISTAYMELSRLRSDDTATYFCAP | VPDRFSGSGSGSDFTLKISWVEAEDVGVYY |
| | GGFAYWGQGTLVTVSS (SEQ ID NO: 230) | CMQATQFPLTFGQGTKLEIK (SEQ ID NO: |
| | | 330) |
| 88 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTNYADDF | NGYTYLYWFLQKPGQSPQLLIYRVSNLASG |
| | KGRVTMTRDTSISTAYMELSRLRSDDTATYFCAP | VPDRFSGSGSGSDFTLKISWVEAEDVGVYY |
| | GGFAYWGQGTLVTVSS (SEQ ID NO: 231) | CMQATQFPLTFGAGTKLEIK (SEQ ID NO: |
| | | 331) |
| 89 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFQ | NGYTYLYWYLQKPGQSPQLLIYRVSNLAS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 232) | YCMQATQFPLTFGQGTKLEIK (SEQ ID NO: |
| | | 332) |
| 90 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFQ | NGYTYLYWYLQKPGQSPQLLIYRVSNHLS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 233) | YCMQATQFPLTFGAGTKLEIK (SEQ ID NO: |
| | | 333) |
| 91 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFQ | NGYTYLYWYLQKPGQSPQLLIYRVSNLLSG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 234) | CMQATQFPLTFGQGTKLEIK (SEQ ID NO: |
| | | 334) |
| 92 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFQ | NGYTYLYWFLQKPGQSPQLLIYRVSNLASG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 235) | CMQATQFPLTFGAGTKLEIK (SEQ ID NO: |
| | | 335) |
| 93 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFK | NGYTYLYWYLQKPGQSPQLLIYRVSNLAS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 236) | YCMQATQFPLTFGQGTKLEIK (SEQ ID NO: |
| | | 336) |
| 94 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFK | NGYTYLYWYLQKPGQSPQLLIYRVSNHLS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 237) | YCMQATQFPLTFGAGTKLEIK (SEQ ID NO: |
| | | 337) |
| 95 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFK | NGYTYLYWYLQKPGQSPQLLIYRVSNLLSG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 238) | CMQATQFPLTFGQGTKLEIK (SEQ ID NO: |
| | | 338) |
| 96 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFK | NGYTYLYWFLQKPGQSPQLLIYRVSNLASG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 239) | CMQATQFPLTFGAGTKLEIK (SEQ ID NO: |
| | | 339) |
| 97 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFQ | NGYTYLYWYLQKPGQSPQLLIYRVSNLAS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 240) | YCMQATQFPLTFGQGTKLEIK (SEQ ID NO: |
| | | 340) |
| 98 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFQ | NGYTYLYWYLQKPGQSPQLLIYRVSNHLS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 241) | YCMQATQFPLTFGAGTKLEIK (SEQ ID NO: |
| | | 341) |
| 99 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFQ | NGYTYLYWYLQKPGQSPQLLIYRVSNLLSG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 242) | CMQATQFPLTFGQGTKLEIK (SEQ ID NO: |
| | | 342) |
| 100 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFQ | NGYTYLYWFLQKPGQSPQLLIYRVSNLASG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 243) | CMQATQFPLTFGAGTKLEIK (SEQ ID NO: |
| | | 343) |
| 101 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFK | NGYTYLYWYLQKPGQSPQLLIYRVSNLAS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 244) | YCMQATQFPLTFGQGTKLEIK (SEQ ID NO: |
| | | 344) |
| 102 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFK | NGYTYLYWYLQKPGQSPQLLIYRVSNHLS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 245) | YCMQATQFPLTFGAGTKLEIK (SEQ ID NO: |
| | | 345) |
| 103 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFK | NGYTYLYWYLQKPGQSPQLLIYRVSNLLSG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 246) | CMQATQFPLTFGQGTKLEIK (SEQ ID NO: |
| | | 346) |
| 104 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFK | NGYTYLYWFLQKPGQSPQLLIYRVSNLASG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 247) | CMQATQFPLTFGAGTKLEIK (SEQ ID NO: |
| | | 347) |
| 105 | EVQLVESGGGLVKPGGSLRLSCAASGFTFSSYSM | DIVMTQSPLSLPVTPGEPASISCRSSQSLLHS |
| | SWVRQAPDKGLEWVATISSSSSYIYYPDSVKGRF | NGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GNYSYYGFDYWGQGTLVTVSS (SEQ ID NO: 248) | YCMQALQTPYTFGQGTKLEIK (SEQ ID NO: |
| | | 348) |
| 106 | EVQLVESGGGLVKPGGSLRLSCAASGFTFSSYSM | DIVMTQSPLSLPVTPGEPASISCRSSQSLLHS |
| | SWVRQAPDKGLEWVATISSSSSYIYYPDSVKGRF | NGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GNYSYYGFDYWGQGTLVTVSS (SEQ ID NO: 249) | YCMQALQTPLTFGQGTKLEIK (SEQ ID NO: |
| | | 349) |
| 107 | EVQLVESGGGLVKPGGSLRLSCAASGFTFSSYSM | DIVMTQSPLSLPVTPGEPASISCRSSQSLLHS |
| | NWVRQAPDKGLEWVSSISSSSSYIYYPDSVKGRF | NGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GNYSYYGFDYWGQGTLVTVSS (SEQ ID NO: 250) | YCMQALQTPYTFGQGTKLEIK (SEQ ID NO: |
| | | 350) |
| 108 | EVQLVESGGGLVKPGGSLRLSCAASGFTFSSYSM | DIVMTQSPLSLPVTPGEPASISCRSSQSLLHS |
| | NWVRQAPDKGLEWVSSISSSSSYIYYPDSVKGRF | NGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GNYSYYGFDYWGQGTLVTVSS (SEQ ID NO: 251) | YCMQALQTPLTFGQGTKLEIK (SEQ ID NO: |
| | | 351) |
| 109 | EVQLVESGGGLVKPGGSLRLSCAASGFTFSSYSM | DIVMTQSPLSLPVTPGEPASISCRSSQSLLHS |
| | SWVRQAPDKGLEWVSSISSSSSYIYYADSVKGRF | NGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GNYSYYGFDYWGQGTLVTVSS (SEQ ID NO: 252) | YCMQALQTPYTFGQGTKLEIK (SEQ ID NO: |
| | | 352) |
| 110 | EVQLVESGGGLVKPGGSLRLSCAASGFTFSSYSM | DIVMTQSPLSLPVTPGEPASISCRSSQSLLHS |
| | SWVRQAPDKGLEWVSSISSSSSYIYYADSVKGRF | NGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GNYSYYGFDYWGQGTLVTVSS (SEQ ID NO: 253) | YCMQALQTPLTFGQGTKLEIK (SEQ ID NO: |
| | | 353) |
| 111 | QIQLVQSGSELKKPGASVKVSCKASGYTFTDYSM | DIVMTQSPLSLPVTPGEPASISCRSSKSLLHS |
| | HWVRQAPGQGLEWMGWINTETGEPTYAQGFTG | NGNTYLYWFLQKPGQSPQLLIYRMSNLAS |
| | RFVFSLDTSVSTAYLQISSLKAEDTAVYFCARGGF | GVPDRFSGSGSGTDFTLKISRVEAEDVGVY |
| | AYWGQGTLVTVSS (SEQ ID NO: 254) | YCMQHLEYPLTFGAGTKLELK (SEQ ID NO: |
| | | 354) |
| 112 | QIQLVQSGSELKKPGASVKVSCKASGYTFTDYSM | DIVMTQSPLSLPVTPGEPASISCRSSKSLLHS |
| | HWVRQAPGQGLEWMGWINTETGEPTYAQGFTG | NGNTYLYWFLQKPGQSPQLLIYRLSNLASG |
| | RFVFSLDTSVSTAYLQISSLKAEDTAVYFCARGGF | VPDRFSGSGSGTDFTLKISRVEAEDVGVYY |
| | AYWGQGTLVTVSS (SEQ ID NO: 255) | CMQHLEYPLTFGAGTKLELK (SEQ ID NO: |
| | | 355) |
| 113 | QIQLVQSGSELKKPGASVKVSCKASGYTFTDYSM | DIVMTQSPLSLPVTPGEPASISCRSSKSLLHS |
| | HWVRQAPGQGLEWMGWIQTETGEPTYAQGFTG | NGNTYLYWFLQKPGQSPQLLIYRMSNLAS |
| | RFVFSLDTSVSTAYLQISSLKAEDTAVYFCAPGGF | GVPDRFSGSGSGTDFTLKISRVEAEDVGVY |
| | AYWGQGTLVTVSS (SEQ ID NO: 256) | YCMQHLEYPLTFGAGTKLELK (SEQ ID NO: |
| | | 356) |
| 114 | QIQLVQSGSELKKPGASVKVSCKASGYTFTDYSM | DIVMTQSPLSLPVTPGEPASISCRSSKSLLHS |
| | HWVRQAPGQGLEWMGWIQTETGEPTYAQGFTG | NGNTYLYWFLQKPGQSPQLLIYRLSNLASG |
| | RFVFSLDTSVSTAYLQISSLKAEDTAVYFCAPGGF | VPDRFSGSGSGTDFTLKISRVEAEDVGVYY |
| | AYWGQGTLVTVSS (SEQ ID NO: 257) | CMQHLEYPLTFGAGTKLELK (SEQ ID NO: |
| | | 357) |
| 115 | QIQLVQSGSELKKPGASVKVSCKASGYTFTDYSM | DIVMTQSPLSLPVTPGEPASISCRSSKSLLHS |
| | HWVRQAPGQGLEWMGWIQTETGEPTYAQGFTG | NGNTYLYWFLQKPGQSPQLLIYRMSNLAS |
| | RFVFSLDTSVSTAYLQISSLKAEDTAVYFCARGGF | GVPDRFSGSGSGTDFTLKISRVEAEDVGVY |
| | AYWGQGTLVTVSS (SEQ ID NO: 258) | YCMQHLEYPLTFGAGTKLELK (SEQ ID NO: |
| | | 358) |
| 116 | QIQLVQSGSELKKPGASVKVSCKASGYTFTDYSM | DIVMTQSPLSLPVTPGEPASISCRSSKSLLHS |
| | HWVRQAPGQGLEWMGWIQTETGEPTYAQGFTG | NGNTYLYWFLQKPGQSPQLLIYRLSNLASG |
| | RFVFSLDTSVSTAYLQISSLKAEDTAVYFCARGGF | VPDRFSGSGSGTDFTLKISRVEAEDVGVYY |
| | AYWGQGTLVTVSS (SEQ ID NO: 259) | CMQHLEYPLTFGAGTKLELK (SEQ ID NO: |
| | | 359) |
| 117 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFK | NGYTYLYWYLQKPGQSPQLLIYRVSNHLS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 260) | YCMQATQFPLTFGAGTKLEIK (SEQ ID NO: |
| | | 360) |
| 118 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFK | NGYTYLYWYLQKPGQSPQLLIYRLSNHLSG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 261) | CMQATQFPLTFGAGTKLEIK (SEQ ID NO: |
| | | 361) |
| 119 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFK | NGYTYLYWYLQKPGQSPQLLIYRVSNHLS |
| | GRVTMTRDTSISTAYMELSRLRSDDTATYFCARG | GVPDRFSGSGSGSDFTLKISWVEAEDVGVY |
| | GFAYWGQGTLVTVSS (SEQ ID NO: 262) | YCMQATQFPLTFGAGTKLEIK (SEQ ID NO: |
| | | 362) |
| 120 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFK | NGYTYLYWYLQKPGQSPQLLIYRLSNHLSG |
| | GRVTMTRDTSISTAYMELSRLRSDDTATYFCARG | VPDRFSGSGSGSDFTLKISWVEAEDVGVYY |
| | GFAYWGQGTLVTVSS (SEQ ID NO: 263) | CMQATQFPLTFGAGTKLEIK (SEQ ID NO: |
| | | 363) |
| 121 | QVQLVQSGAEVKKPGASVKVSCKASGYTFTDYS | DIVMTQTPPSLPVNPGEPASISCRSSQSLLHS |
| | MHWVRQAPGQGLKWMGWIQTETGEPTYADDFK | NGYTYLYWYLQKPGQSPQLLIYRVSNHLS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 264) | YCMQATQFPLTFGAGTKLEIK (SEQ ID NO: |
| | | 364) |
| 122 | QVQLVQSGAEVKKPGASVKVSCKASGYTFTDYS | DIVMTQTPPSLPVNPGEPASISCRSSQSLLHS |
| | MHWVRQAPGQGLKWMGWIQTETGEPTYADDFK | NGYTYLYWYLQKPGQSPQLLIYRLSNHLSG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 265) | CMQATQFPLTFGAGTKLEIK (SEQ ID NO: |
| | | 365) |
| 123 | QVQLVQSGAEVKKPGASVKVSCKASGYTFTDYS | DIVMTQTPPSLPVNPGEPASISCRSSQSLLHS |
| | MHWVRQAPGQGLKWMGWIQTETGEPTYADDFK | NGYTYLYWYLQKPGQSPQLLIYRVSNHLS |
| | GRVTMTRDTSISTAYMELSRLRSDDTATYFCARG | GVPDRFSGSGSGSDFTLKISWVEAEDVGVY |
| | GFAYWGQGTLVTVSS (SEQ ID NO: 266) | YCMQATQFPLTFGAGTKLEIK (SEQ ID NO: |
| | | 366) |
| 124 | QVQLVQSGAEVKKPGASVKVSCKASGYTFTDYS | DIVMTQTPPSLPVNPGEPASISCRSSQSLLHS |
| | MHWVRQAPGQGLKWMGWIQTETGEPTYADDFK | NGYTYLYWYLQKPGQSPQLLIYRLSNHLSG |
| | GRVTMTRDTSISTAYMELSRLRSDDTATYFCARG | VPDRFSGSGSGSDFTLKISWVEAEDVGVYY |
| | GFAYWGQGTLVTVSS (SEQ ID NO: 267) | CMQATQFPLTFGAGTKLEIK (SEQ ID NO: |
| | | 367) |
| 125 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFQ | NGYTYLYWYLQKPGQSPQLLIYRVSNLAS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 268) | YCMQATQFPLTFGQGTKLEIK (SEQ ID NO: |
| | | 368) |
| 126 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFQ | NGYTYLYWYLQKPGQSPQLLIYRLSNLASG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 269) | CMQATQFPLTFGQGTKLEIK (SEQ ID NO: |
| | | 369) |
| 127 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFQ | NGYTYLYWYLQKPGQSPQLLIYRVSNLAS |
| | GRVTMTRDTSISTAYMELSRLRSDDTATYFCARG | GVPDRFSGSGSGSDFTLKISWVEAEDVGVY |
| | GFAYWGQGTLVTVSS (SEQ ID NO: 270) | YCMQATQFPLTFGQGTKLEIK (SEQ ID NO: |
| | | 370) |
| 128 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFQ | NGYTYLYWYLQKPGQSPQLLIYRLSNLASG |
| | GRVTMTRDTSISTAYMELSRLRSDDTATYFCARG | VPDRFSGSGSGSDFTLKISWVEAEDVGVYY |
| | GFAYWGQGTLVTVSS (SEQ ID NO: 271) | CMQATQFPLTFGQGTKLEIK (SEQ ID NO: |
| | | 371) |
| 129 | QVQLVQSGAEVKKPGASVKVSCKASGYTFTDYS | DIVMTQTPPSLPVNPGEPASISCRSSQSLLHS |
| | MHWVRQAPGQGLEWMGWIQTETGEPTYADDFQ | NGYTYLYWYLQKPGQSPQLLIYNLASGVP |
| | GRVTMTRDTSISTAYMELSRLRSDDTATYFCAPG | DRFSGSGSGSDFTLKISWVEAEDVGVYYC |
| | GFAYWGQGTLVTVSS (SEQ ID NO: 272) | MQATQFPLTFRVSGQGTKLEIK (SEQ ID |
| | | NO: 372) |
| 130 | QVQLVQSGAEVKKPGASVKVSCKASGYTFTDYS | DIVMTQTPPSLPVNPGEPASISCRSSQSLLHS |
| | MHWVRQAPGQGLEWMGWIQTETGEPTYADDFQ | NGYTYLYWYLQKPGQSPQLLIYRLSNLASG |
| | GRVTMTRDTSISTAYMELSRLRSDDTATYFCAPG | VPDRFSGSGSGSDFTLKISWVEAEDVGVYY |
| | GFAYWGQGTLVTVSS (SEQ ID NO: 273) | CMQATQFPLTFGQGTKLEIK (SEQ ID NO: |
| | | 373) |
| 131 | QVQLVQSGAEVKKPGASVKVSCKASGYTFTDYS | DIVMTQTPPSLPVNPGEPASISCRSSQSLLHS |
| | MHWVRQAPGQGLEWMGWIQTETGEPTYADDFQ | NGYTYLYWYLQKPGQSPQLLIYRVSNLAS |
| | GRVTMTRDTSISTAYMELSRLRSDDTATYFCARG | GVPDRFSGSGSGSDFTLKISWVEAEDVGVY |
| | GFAYWGQGTLVTVSS (SEQ ID NO: 274) | YCMQATQFPLTFGQGTKLEIK (SEQ ID NO: |
| | | 374) |
| 132 | QVQLVQSGAEVKKPGASVKVSCKASGYTFTDYS | DIVMTQTPPSLPVNPGEPASISCRSSQSLLHS |
| | MHWVRQAPGQGLEWMGWIQTETGEPTYADDFQ | NGYTYLYWYLQKPGQSPQLLIYRLSNLASG |
| | GRVTMTRDTSISTAYMELSRLRSDDTATYFCARG | VPDRFSGSGSGSDFTLKISWVEAEDVGVYY |
| | GFAYWGQGTLVTVSS (SEQ ID NO: 275) | CMQATQFPLTFGQGTKLEIK (SEQ ID NO: |
| | | 375) |
| 133 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFK | NGYTYLYWYLQKPGQSPQLLIYRMSNHLS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 276) | YCMQATQFPLTFGAGTKLEIK (SEQ ID NO: |
| | | 376) |
| 134 | QVQLVQSGAEVKKPGASVKVSCKASGYTFTDYS | DIVMTQTPPSLPVNPGEPASISCRSSQSLLHS |
| | MHWVRQAPGQGLKWMGWINTETGEPTYADDFK | NGYTYLYWYLQKPGQSPQLLIYRMSNHLS |
| | GRVTMTRDTSISTAYMELSRLRSDDTATYFCARG | GVPDRFSGSGSGSDFTLKISWVEAEDVGVY |
| | GFAYWGQGTLVTVSS (SEQ ID NO: 277) | YCMQATQFPLTFGAGTKLEIK (SEQ ID NO: |
| | | 377) |
| 135 | QVQLVQSGAEVKKPGASVKVSCKASGYTFTDYS | DIVMTQTPPSLPVNPGEPASISCRSSQSLLHS |
| | MHWVRQAPGQGLKWMGWIQTETGEPTYADDFK | NGYTYLYWYLQKPGQSPQLLIYRMSNHLS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 278) | YCMQATQFPLTFGAGTKLEIK (SEQ ID NO: |
| | | 378) |
| 136 | QVQLVQSGAEVKKPGASVKVSCKASGYTFTDYS | DIVMTQTPPSLPVNPGEPASISCRSSQSLLHS |
| | MHWVRQAPGQGLKWMGWIQTETGEPTYADDFK | NGYTYLYWYLQKPGQSPQLLIYRMSNHLS |
| | GRVTMTRDTSISTAYMELSRLRSDDTATYFCARG | GVPDRFSGSGSGSDFTLKISWVEAEDVGVY |
| | GFAYWGQGTLVTVSS (SEQ ID NO: 279) | YCMQATQFPLTFGAGTKLEIK (SEQ ID NO: |
| | | 379) |
| 137 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFQ | NGYTYLYWYLQKPGQSPQLLIYRMSNLAS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 280) | YCMQATQFPLTFGQGTKLEIK (SEQ ID NO: |
| | | 380) |
| 138 | QVQLVQSGAEVKKPGASVKVSCKASGYTFTDYS | DIVMTQTPPSLPVNPGEPASISCRSSQSLLHS |
| | MHWVRQAPGQGLEWMGWINTETGEPTYADDFQ | NGYTYLYWYLQKPGQSPQLLIYRMSNLAS |
| | GRVTMTRDTSISTAYMELSRLRSDDTATYFCARG | GVPDRFSGSGSGSDFTLKISWVEAEDVGVY |
| | GFAYWGQGTLVTVSS (SEQ ID NO: 281) | YCMQATQFPLTFGQGTKLEIK (SEQ ID NO: |
| | | 381) |
| 139 | QVQLVQSGAEVKKPGASVKVSCKASGYTFTDYS | DIVMTQTPPSLPVNPGEPASISCRSSQSLLHS |
| | MHWVRQAPGQGLEWMGWIQTETGEPTYADDFQ | NGYTYLYWYLQKPGQSPQLLIYRMSNLAS |
| | GRVTMTRDTSISTAYMELSRLRSDDTATYFCAPG | GVPDRFSGSGSGSDFTLKISWVEAEDVGVY |
| | GFAYWGQGTLVTVSS (SEQ ID NO: 282) | YCMQATQFPLTFGQGTKLEIK (SEQ ID NO: |
| | | 382) |
| 140 | QVQLVQSGAEVKKPGASVKVSCKASGYTFTDYS | DIVMTQTPPSLPVNPGEPASISCRSSQSLLHS |
| | MHWVRQAPGQGLEWMGWIQTETGEPTYADDFQ | NGYTYLYWYLQKPGQSPQLLIYRMSNLAS |
| | GRVTMTRDTSISTAYMELSRLRSDDTATYFCARG | GVPDRFSGSGSGSDFTLKISWVEAEDVGVY |
| | GFAYWGQGTLVTVSS (SEQ ID NO: 283) | YCMQATQFPLTFGQGTKLEIK (SEQ ID NO: |
| | | 383) |
| 141 | EVQLVESGGGLVKPGGSLRLSCAASGFTFSRYGM | DIVMTQSPLSLPVTPGEPASISCRSSKSLLHS |
| | SWVRQAPGKGLEWVSSISIGGTYTYYADSVKGRF | NGITYLYWYLQKPGQSPQLLIYQMSNLASG |
| | TISRDNAKNSLYLQMNSLRAEDTAVYYCARRGY | VPDRFSGSGSGTDFTLKISRVEAEDVGVYY |
| | GQYSYYGMDYWGQGTLVTVSS (SEQ ID NO: | CAQNLELPLTFGQGTKLEIK (SEQ ID NO: |
| | 284) | 384) |
| 142 | EVQLVESGGGLVKPGGSLRLSCAASGFTFSRYGM | DIVMTQSPLSLPVTPGEPASISCRSSKSLLHS |
| | SWVRQAPGKGLEWVSSISIGGTYTYYADSVKGRF | QGITYLYWYLQKPGQSPQLLIYQMSNLASG |
| | TISRDNAKNSLYLQMNSLRAEDTAVYYCARRGY | VPDRFSGSGSGTDFTLKISRVEAEDVGVYY |
| | GQYSYYGMDYWGQGTLVTVSS (SEQ ID NO: | CAQNLELPLTFGQGTKLEIK (SEQ ID NO: |
| | 285) | 385) |
| 143 | EVQLVESGGGLVKPGGSLRLSCAASGFTFSRYGM | DIVMTQSPLSLPVTPGEPASISCRSSKSLLHS |
| | SWVRQAPGKGLEWVSSISIGGTYTYYADSVKGRF | NGITYLYWYLQKPGQSPQLLIYQMSNLASG |
| | TISRDNAKNSLYLQMNSLRAEDTAVYYCARRGY | VPDRFSGSGSGTDFTLKISRVEAEDVGVYY |
| | GQYSYYGLDYWGQGTLVTVSS (SEQ ID NO: 286) | CAQNLELPLTFGQGTKLEIK (SEQ ID NO: |
| | | 386) |
| 144 | EVQLVESGGGLVKPGGSLRLSCAASGFTFSRYGM | DIVMTQSPLSLPVTPGEPASISCRSSKSLLHS |
| | SWVRQAPGKGLEWVSSISIGGTYTYYADSVKGRF | QGITYLYWYLQKPGQSPQLLIYQMSNLASG |
| | TISRDNAKNSLYLQMNSLRAEDTAVYYCARRGY | VPDRFSGSGSGTDFTLKISRVEAEDVGVYY |
| | GQYSYYGLDYWGQGTLVTVSS (SEQ ID NO: 287) | CAQNLELPLTFGQGTKLEIK (SEQ ID NO: |
| | | 387) |
| 145 | EVQLVESGGGLVKPGGSLRLSCAASGFTFSSYSM | DIVMTQSPLSLPVTPGEPASISCRSSQSLLHS |
| | SWVRQAPDKGLEWVSSISSSSSYIYYADSVKGRF | NGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GQYSYYGFDYWGQGTLVTVSS (SEQ ID NO: 288) | YCMQALQTPYTFGQGTKLEIK (SEQ ID NO: |
| | | 388) |
| 146 | EVQLVESGGGLVKPGGSLRLSCAASGFTFSSYSM | DIVMTQSPLSLPVTPGEPASISCRSSQSLLHS |
| | SWVRQAPDKGLEWVSSISSSSSYIYYADSVKGRF | QGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GQYSYYGFDYWGQGTLVTVSS (SEQ ID NO: 289) | YCMQALQTPYTFGQGTKLEIK (SEQ ID NO: |
| | | 389) |
| 147 | EVQLVESGGGLVKPGGSLRLSCAASGFTFSSYSM | DIVMTQSPLSLPVTPGEPASISCRSSQSLLHS |
| | SWVRQAPDKGLEWVSSISSSSSYIYYADSVKGRF | NGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GQYSYYGLDYWGQGTLVTVSS (SEQ ID NO: 290) | YCMQALQTPYTFGQGTKLEIK (SEQ ID NO: |
| | | 390) |
| 148 | EVQLVESGGGLVKPGGSLRLSCAASGFTFSSYSM | DIVMTQSPLSLPVTPGEPASISCRSSQSLLHS |
| | SWVRQAPDKGLEWVSSISSSSSYIYYADSVKGRF | QGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GQYSYYGLDYWGQGTLVTVSS (SEQ ID NO: 291) | YCMQALQTPYTFGQGTKLEIK (SEQ ID NO: |
| | | 391) |
| 149 | EVQLVESGGGLVQPGGSLRLSCAASGFTFSRYGM | DIVMTQSALSNPVTLGQSASISCRSSKSLLH |
| | SWVRQAPGKRLEWVATISIGGTYTYYPDSMKGR | SQGITYLYWYLQKPGQSPQLLIYQMSNRAS |
| | FTISRDNSKNTLYLQMNSLRAEDTAMYYCARRG | GVPDRFSSSGSGTDFTLKISRVEAEDVGVY |
| | YGQYSYYGMDYWGQGTLVTVSS (SEQ ID NO: | YCAQNLELPLTFGQGTKLEIK (SEQ ID NO: |
| | 292) | 392) |
| 150 | EVQLVESGGGLVKPGGSLRLSCAASGFTFSSYSM | DIVMTQSPLSLPVTPGEPASISCRSSQSLLHS |
| | SWVRQAPDKGLEWVSSISSSSSYIYYADSVKGRF | NGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GQYSYYGMDYWGQGTLVTVSS (SEQ ID NO: | YCMQALQTPYTFGQGTKLEIK (SEQ ID NO: |
| | 293) | 393) |
| 151 | EVQLVESGGGLVKPGGSLRLSCAASGFTFSSYSM | DIVMTQSPLSLPVTPGEPASISCRSSQSLLHS |
| | SWVRQAPDKGLEWVSSISSSSSYIYYADSVKGRF | QGYNYLYWYLQKPGQSPQLLIYLGSNLAS |
| | TISRDNAKNSLYLQMNSLRAEDTAVYYCARRGY | GVPDRFSGSGSGTDFTLKISRVEAEDVGVY |
| | GQYSYYGMDYWGQGTLVTVSS (SEQ ID NO: | YCMQALQTPYTFGQGTKLEIK (SEQ ID NO: |
| | 294) | 394) |

In some embodiments, the HCDR1 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 104 (GFTFSRYG). In some embodiments, the HCDR1 comprises an amino acid sequence according to SEQ ID NO: 104 (GFTFSRYG). In some embodiments, the HCDR2 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 115 (ISIGGTYT). In some embodiments, the HCDR2 comprises an amino acid sequence according to SEQ ID NO: 115 (ISIGGTYT). In some embodiments, the HCDR3 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 134 (ARRGYGX₅YSYYGX₁₁DY, wherein X₅ is N, S, or Q; and X₁₁ is M, L, I, or V). In some embodiments, the HCDR3 comprises an amino acid sequence according to SEQ ID NO: 134 (ARRGYGX₅YSYYGX₁₁DY, wherein X₅ is N, S, or Q; and X₁₁ is M, L, I, orV).

In some embodiments, the LCDR1 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 135 (KSLLHSX₁₀GITY, wherein X₁₀ is N, V, D, Q, S or E). In some embodiments, the LCDR1 comprises an amino acid sequence according to SEQ ID NO: 135 (KSLLHSX₁₀GITY, wherein X₁₀ is N, V, D, Q, S or E). In some embodiments, the LCDR2 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 136 (QX₂S, wherein X₂ is M, L, V, or I). In some embodiments, the LCDR2 comprises an amino acid sequence according to SEQ ID NO: 136 (QX₂S, wherein X₂ is M, L, V, or I). In some embodiments, the LCDR3 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 133 (AQNLELPLT). In some embodiments, the LCDR3 comprises an amino acid sequence according to SEQ ID NO: 133 (AQNLELPLT).

In some embodiments, the HCDR3 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of SEQ ID NOs: 125-131. In some embodiments, the HCDR3 comprises an amino acid sequence of any one of SEQ ID NOs: 125-131. In some embodiments, the LCDR1 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of SEQ ID NOs: 107-112. In some embodiments, the LCDR1 comprises an amino acid sequence according to any one of SEQ ID NOs: 107-112. In some embodiments, the LCDR2 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of SEQ ID NOs: 120-123. In some embodiments, the LCDR2 comprises an amino acid sequence according to any one of SEQ ID NOs: 120-123.

In some embodiments, the HCDR3 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 131, the LCDR1 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 111, and the LCDR2 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 120. In some embodiments, the HCDR3 comprises an amino acid sequence according to SEQ ID NO: 131, the LCDR1 comprises an amino acid sequence according to SEQ ID NO: 111, and the LCDR2 comprises an amino acid sequence according to SEQ ID NO: 120.

In some embodiments, the HCDR1 comprises an amino acid sequence according to SEQ ID NO: 104 (GFTFSRYG), the HCDR2 comprises an amino acid sequence according to SEQ ID NO: 115 (ISIGGTYT), the HCDR3 comprises an amino acid sequence according to SEQ ID NO: 131 (ARRGYGQYSYYGLDY), the LCDR1 comprises an amino acid sequence according to SEQ ID NO: 111 (KSLLHSQGITY), the LCDR2 comprises an amino acid sequence according to SEQ ID NO: 120 (QMS), and the LCDR3 comprises an amino acid sequence according to SEQ ID NO: 133 (AQNLELPLT).

In some embodiments, the antibody or antigen binding fragment comprises a heavy chain variable region comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of SEQ ID Nos: 16-39, 43-46, and 49-51. In some embodiments, the antibody or antigen binding fragment comprises a heavy chain variable region having at least about 90% identity to any one of SEQ ID Nos: 16-39, 43-46, and 49-51.

In some embodiments, the antibody or antigen binding fragment comprises a light chain variable region comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of SEQ ID Nos: 67-90, 94-97, and 100-102. In some embodiments, the antibody or antigen binding fragment comprises a light chain variable region having at least about 90% identity to any one of SEQ ID Nos: 67-90, 94-97, and 100-102.

In some embodiments, the antibody or antigen binding fragment comprises a heavy chain variable region comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 51. In some embodiments, the antibody or antigen binding fragment comprises a heavy chain variable region having at least about 90% identity to SEQ ID NO: 51.

In some embodiments, the antibody or antigen binding fragment comprises a light chain variable region comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 102. In some embodiments, the antibody or fragment comprises a light chain variable region having at least about 90% identity to SEQ ID NO: 102.

In some embodiments, the antibody or antigen binding fragment comprises a heavy chain variable region comprising an amino acid sequence having at least about 90% identity to SEQ ID NO: 51, and a light chain variable region comprising an amino acid sequence having at least about 90% identity to SEQ ID NO: 102. In some embodiments, the antibody or antigen binding fragment comprises a heavy chain variable region comprising the amino acid sequence of SEQ ID NO: 51, and a light chain variable region comprising the amino acid sequence of SEQ ID NO: 102.

In some embodiments, the antibody or fragment comprises a heavy chain amino acid sequence according to SEQ ID NO: 186 and a light chain amino acid sequence according to SEQ ID NO: 187.

In some embodiments, the HCDR1 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 103 (GYTFTDYS). In some embodiments, the HCDR2 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 139 (IX₁₂TETGEP, wherein X₁₂ is N or Q). In some embodiments, the HCDR3 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 124 (APGGFAY). In some embodiments, the LCDR1 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 140 (KSLLHSX₁₃GNTY, wherein X₁₃ is N or S). In some embodiments, the LCDR2 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 141 (RX₁₄S, wherein X₁₄ is M, V, L, or I). In some embodiments, LCDR3 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 132 (MQHLEYPLT).

In some embodiments, the HCDR1 comprises an amino acid sequence according to SEQ ID NO: 103 (GYTFTDYS). In some embodiments, the HCDR2 comprises an amino acid sequence according to SEQ ID NO: 139 (IX₁₂TETGEP, wherein X₁₂ is N or Q). In some embodiments, the HCDR3 comprises an amino acid sequence according to SEQ ID NO: 124 (APGGFAY). In some embodiments, the LCDR1 comprises an amino acid sequence according to SEQ ID NO: 140 (KSLLHSX₁₃GNTY, wherein X₁₃ is N or S). In some embodiments, the LCDR2 comprises an amino acid sequence according to SEQ ID NO: 141 (RX₁₄S, wherein X₁₄ is M, V, L, or I). In some embodiments, LCDR3 comprises an amino acid sequence according to SEQ ID NO: 132 (MQHLEYPLT).

In some embodiments, the HCDR2 comprises the amino acid sequence of SEQ ID NO: 113 or 114. In some embodiments, the LCDR1 comprises the amino acid sequence of SEQ ID NO: 105 or 106. In some embodiments, the LCDR2 comprises the amino acid sequence of any one of SEQ ID NOs: 116-119.

In some embodiments, the antibody or antigen binding fragment comprises a heavy chain variable region comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of SEQ ID Nos: 1-16, 40-42, 47 and 48. In some embodiments, the antibody or antigen binding fragment comprises a heavy chain variable region having at least about 90% identity to any one of SEQ ID Nos: 1-16, 40-42, 47 and 48.

In some embodiments, the antibody or antigen binding fragment comprises a light chain variable region comprising an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of SEQ ID Nos: 52-66, 91-93, 98 and 99. In some embodiments, the antibody or antigen binding fragment comprises a light chain variable region having at least about 90% identity to any one of SEQ ID Nos: 52-66, 91-93, 98 and 99.

In some embodiments, the HCDR1 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 103 or 104. In some embodiments, the HCDR2 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 113, 114, or 115. In some embodiments, the HCDR3 comprises an amino acid sequence an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of SEQ ID Nos: 124-131. In some embodiments, the LCDR1 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of SEQ ID Nos: 105-112. In some embodiments, the LCDR2 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to any one of SEQ ID Nos 116-123. In some embodiments, the LCDR3 comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 132 or 133.

In some embodiments, the HCDR1 comprises the amino acid sequence of SEQ ID NO: 103 or 104. In some embodiments, the HCDR2 comprises the amino acid sequence of SEQ ID NO: 113, 114, or 115. In some embodiments, the HCDR3 comprises an amino acid sequence selected from the group consisting of SEQ ID Nos: 124-131. In some embodiments, the LCDR1 comprises an amino acid sequence selected from the group consisting of SEQ ID Nos: 105-112.

In some embodiments, the LCDR2 comprises an amino acid sequence selected from the group consisting of SEQ ID Nos 116-123. In some embodiments, the LCDR3 comprises the amino acid sequence of SEQ ID NO: 132 or 133.

In some embodiments, the HCDR1 comprises the amino acid sequence of SEQ ID NO: 103 or 104, the HCDR2 comprises the amino acid sequence of SEQ ID NO: 113, 114, or 115, the HCDR3 comprises an amino acid sequence selected from the group consisting of SEQ ID Nos: 124-131, the LCDR1 comprises an amino acid sequence selected from the group consisting of SEQ ID Nos: 105-112, the LCDR2 comprises an amino acid sequence selected from the group consisting of SEQ ID Nos 116-123, and the LCDR3 comprises the amino acid sequence of SEQ ID NO: 132 or 133.

In some embodiments, the HCDR1, HCDR2, and HCDR3 comprise the amino acid sequence of SEQ ID NOs: 103, 113, and 124, respectively, and LCDR1, LCDR2, and LCDR3 comprise the amino acid sequence of SEQ ID NOs: 105, 116, and 132, respectively. In some embodiments, the HCDR1, HCDR2, and HCDR3 comprise the amino acid sequence of SEQ ID NOs: 103, 114, and 124, respectively, and LCDR1, LCDR2, and LCDR3 comprise the amino acid sequence of SEQ ID NOs: 105, 116, and 132, respectively. In some embodiments, the HCDR1, HCDR2, and HCDR3 comprise the amino acid sequence of SEQ ID NOs: 103, 113, and 124, respectively, and LCDR1, LCDR2, and LCDR3 comprise the amino acid sequence of SEQ ID NOs: 106, 116, and 132, respectively.

In some embodiments, the HCDR1, HCDR2, and HCDR3 comprise the amino acid sequence of SEQ ID NOs: 103, 113, and 124, respectively, and LCDR1, LCDR2, and LCDR3 comprise the amino acid sequence of SEQ ID NOs: 105, 117, and 132, respectively. In some embodiments, the HCDR1, HCDR2, and HCDR3 comprise the amino acid sequence of SEQ ID NOs: 103, 113, and 124, respectively, and LCDR1, LCDR2, and LCDR3 comprise the amino acid sequence of SEQ ID NOs: 105, 118, and 132, respectively. In some embodiments, the HCDR1, HCDR2, and HCDR3 comprise the amino acid sequence of SEQ ID NOs: 103, 113, and 124, respectively, and LCDR1, LCDR2, and LCDR3 comprise the amino acid sequence of SEQ ID NOs: 105, 119, and 132, respectively.

In some embodiments, the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 107, 120, and 133, respectively. In some embodiments, the HCDR1, HCDR2, and HCDR3 comprise the amino acid sequence of SEQ ID NOs: 104, 115, and 126, respectively, and LCDR1, LCDR2, and LCDR3 comprise the amino acid sequence of SEQ ID NOs: 107, 120, and 133, respectively. In some embodiments, the HCDR1, HCDR2, and HCDR3 comprise the amino acid sequence of SEQ ID NOs: 104, 115, and 127, respectively, and LCDR1, LCDR2, and LCDR3 comprise the amino acid sequence of SEQ ID NOs: 107, 120, and 133, respectively.

In some embodiments, the HCDR1, HCDR2, and HCDR3 comprise the amino acid sequence of SEQ ID NOs: 104, 115, and 128, respectively, and LCDR1, LCDR2, and LCDR3 comprise the amino acid sequence of SEQ ID NOs: 107, 120, and 133, respectively. In some embodiments, the HCDR1, HCDR2, and HCDR3 comprise the amino acid sequence of SEQ ID NOs: 104, 115, and 129, respectively, and LCDR1, LCDR2, and LCDR3 comprise the amino acid sequence of SEQ ID NOs: 107, 120, and 133, respectively. In some embodiments, the HCDR1, HCDR2, and HCDR3 comprise the amino acid sequence of SEQ ID NOs: 104, 115, and 130, respectively, and LCDR1, LCDR2, and LCDR3 comprise the amino acid sequence of SEQ ID NOs: 107, 120, and 133, respectively.

In some embodiments, the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 108, 120, and 133, respectively. In some embodiments, the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 109, 120, and 133, respectively. In some embodiments, the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 110, 120, and 133, respectively.

In some embodiments, the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 111, 120, and 133, respectively. In some embodiments, the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 112, 120, and 133, respectively. In some embodiments, the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 107, 121, and 133, respectively.

In some embodiments, the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 107, 122, and 133, respectively. In some embodiments, the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 107, 123, and 133, respectively. In some embodiments, the HCDR1, HCDR2, and HCDR3 comprise the amino acid sequence of SEQ ID NOs: 104, 115, and 131, respectively, and LCDR1, LCDR2, and LCDR3 comprise the amino acid sequence of SEQ ID NOs: 107, 120, and 133, respectively. In some embodiments, the HCDR1, HCDR2, and HCDR3 comprise the amino acid sequence of SEQ ID NOs: 104, 115, and 131, respectively, and LCDR1, LCDR2, and LCDR3 comprise the amino acid sequence of SEQ ID NOs: 111, 120, and 133, respectively.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 1 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 52. In some embodiments, the heavy chain variable region of the antibodies or antigen binding fragments disclosed herein comprises the amino acid sequence of SEQ ID NO: 1, and the light chain variable region of the antibodies or antigen binding fragments disclosed herein comprises an amino acid sequence of SEQ ID NO: 52.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 2 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 53. In some embodiments, the heavy chain variable region of the antibodies or antigen binding fragments disclosed herein comprises the amino acid sequence of SEQ ID NO: 2, and the light chain variable region of the antibodies or antigen binding fragments disclosed herein comprises an amino acid sequence of SEQ ID NO: 53.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 3 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 54. In some embodiments, the heavy chain variable region of the antibodies or antigen binding fragments disclosed herein comprises the amino acid sequence of SEQ ID NO: 3, and the light chain variable region of the antibodies or antigen binding fragments disclosed herein comprises an amino acid sequence of SEQ ID NO: 54.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 4 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 55. In some embodiments, the heavy chain variable region of the antibodies or antigen binding fragments disclosed herein comprises the amino acid sequence of SEQ ID NO: 4, and the light chain variable region of the antibodies or antigen binding fragments disclosed herein comprises an amino acid sequence of SEQ ID NO: 55.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 5 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 56. In some embodiments, the heavy chain variable region of the antibodies or antigen binding fragments disclosed herein comprises the amino acid sequence of SEQ ID NO: 5, and the light chain variable region of the antibodies or antigen binding fragments disclosed herein comprises an amino acid sequence of SEQ ID NO: 56.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 6 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 57. In some embodiments, the heavy chain variable region of the antibodies or antigen binding fragments disclosed herein comprises the amino acid sequence of SEQ ID NO: 6, and the light chain variable region of the antibodies or antigen binding fragments disclosed herein comprises an amino acid sequence of SEQ ID NO: 57.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 7 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 58. In some embodiments, the heavy chain variable region of the antibodies or antigen binding fragments disclosed herein comprises the amino acid sequence of SEQ ID NO: 7, and the light chain variable region of the antibodies or antigen binding fragments disclosed herein comprises an amino acid sequence of SEQ ID NO: 58.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 8 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 59. In some embodiments, the heavy chain variable region of the antibodies or antigen binding fragments disclosed herein comprises the amino acid sequence of SEQ ID NO: 8, and the light chain variable region of the antibodies or antigen binding fragments disclosed herein comprises an amino acid sequence of SEQ ID NO: 59.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 9 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 60. In some embodiments, the heavy chain variable region of the antibodies or antigen binding fragments disclosed herein comprises the amino acid sequence of SEQ ID NO: 9, and the light chain variable region of the antibodies or antigen binding fragments disclosed herein comprises an amino acid sequence of SEQ ID NO: 60.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 10 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 61. In some embodiments, the heavy chain variable region of the antibodies or antigen binding fragments disclosed herein comprises the amino acid sequence of SEQ ID NO: 10, and the light chain variable region of the antibodies or antigen binding fragments disclosed herein comprises an amino acid sequence of SEQ ID NO: 61.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 11 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 62. In some embodiments, the heavy chain variable region of the antibodies or antigen binding fragments disclosed herein comprises the amino acid sequence of SEQ ID NO: 11, and the light chain variable region of the antibodies or antigen binding fragments disclosed herein comprises an amino acid sequence of SEQ ID NO: 62.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 12 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 63. In some embodiments, the heavy chain variable region of the antibodies or antigen binding fragments disclosed herein comprises the amino acid sequence of SEQ ID NO: 12, and the light chain variable region of the antibodies or antigen binding fragments disclosed herein comprises an amino acid sequence of SEQ ID NO: 63.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 13 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 64. In some embodiments, the heavy chain variable region of the antibodies or antigen binding fragments disclosed herein comprises the amino acid sequence of SEQ ID NO: 13, and the light chain variable region of the antibodies or antigen binding fragments disclosed herein comprises an amino acid sequence of SEQ ID NO: 64.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 14 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 65. In some embodiments, the heavy chain variable region of the antibodies or antigen binding fragments disclosed herein comprises the amino acid sequence of SEQ ID NO: 14, and the light chain variable region of the antibodies or antigen binding fragments disclosed herein comprises an amino acid sequence of SEQ ID NO: 65.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 15 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 66. In some embodiments, the heavy chain variable region of the antibodies or antigen binding fragments disclosed herein comprises the amino acid sequence of SEQ ID NO: 15, and the light chain variable region of the antibodies or antigen binding fragments disclosed herein comprises an amino acid sequence of SEQ ID NO: 66.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 16 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 67. In some embodiments, the heavy chain variable region of the antibodies or antigen binding fragments disclosed herein comprises the amino acid sequence of SEQ ID NO: 16, and the light chain variable region of the antibodies or antigen binding fragments disclosed herein comprises an amino acid sequence of SEQ ID NO: 67.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 17 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 68. In some embodiments, the heavy chain variable region of the antibodies or antigen binding fragments disclosed herein comprises the amino acid sequence of SEQ ID NO: 17, and the light chain variable region of the antibodies or antigen binding fragments disclosed herein comprises an amino acid sequence of SEQ ID NO: 68.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 18 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 69. In some embodiments, the heavy chain variable region of the antibodies or antigen binding fragments disclosed herein comprises the amino acid sequence of SEQ ID NO: 18, and the light chain variable region of the antibodies or antigen binding fragments disclosed herein comprises an amino acid sequence of SEQ ID NO: 69.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 19 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 70. In some embodiments, the heavy chain variable region of the antibodies or antigen binding fragments disclosed herein comprises the amino acid sequence of SEQ ID NO: 19, and the light chain variable region of the antibodies or antigen binding fragments disclosed herein comprises an amino acid sequence of SEQ ID NO: 70.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 20 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 71. In some embodiments, the heavy chain variable region of the antibodies or antigen binding fragments disclosed herein comprises the amino acid sequence of SEQ ID NO: 20, and the light chain variable region of the antibodies or antigen binding fragments disclosed herein comprises an amino acid sequence of SEQ ID NO: 71.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 21 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 72. In some embodiments, the heavy chain variable region of the antibodies or antigen binding fragments disclosed herein comprises the amino acid sequence of SEQ ID NO: 21, and the light chain variable region of the antibodies or antigen binding fragments disclosed herein comprises an amino acid sequence of SEQ ID NO: 72.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 22 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 73. In some embodiments, the heavy chain variable region of the antibodies or antigen binding fragments disclosed herein comprises the amino acid sequence of SEQ ID NO: 22, and the light chain variable region of the antibodies or antigen binding fragments disclosed herein comprises an amino acid sequence of SEQ ID NO: 73.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 23 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 74. In some embodiments, the heavy chain variable region of the antibodies or antigen binding fragments disclosed herein comprises the amino acid sequence of SEQ ID NO: 23, and the light chain variable region of the antibodies or antigen binding fragments disclosed herein comprises an amino acid sequence of SEQ ID NO: 74.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 24 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 75. In some embodiments, the heavy chain variable region of the antibodies or antigen binding fragments disclosed herein comprises the amino acid sequence of SEQ ID NO: 24, and the light chain variable region of the antibodies or antigen binding fragments disclosed herein comprises an amino acid sequence of SEQ ID NO: 75.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 25 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 76. In some embodiments, the heavy chain variable region of the antibodies or antigen binding fragments disclosed herein comprises the amino acid sequence of SEQ ID NO: 25, and the light chain variable region of the antibodies or antigen binding fragments disclosed herein comprises an amino acid sequence of SEQ ID NO: 76.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 26 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 77. In some embodiments, the heavy chain variable region of the antibodies or antigen binding fragments disclosed herein comprises the amino acid sequence of SEQ ID NO: 26, and the light chain variable region of the antibodies or antigen binding fragments disclosed herein comprises an amino acid sequence of SEQ ID NO: 77.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 27 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 78. In some embodiments, the heavy chain variable region of the antibodies or antigen binding fragments disclosed herein comprises the amino acid sequence of SEQ ID NO: 27, and the light chain variable region of the antibodies or antigen binding fragments disclosed herein comprises an amino acid sequence of SEQ ID NO: 78.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 28 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 79. In some embodiments, the heavy chain variable region of the antibodies or antigen binding fragments disclosed herein comprises the amino acid sequence of SEQ ID NO: 28, and the light chain variable region of the antibodies or antigen binding fragments disclosed herein comprises an amino acid sequence of SEQ ID NO: 79.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 29 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 80. In some embodiments, the heavy chain variable region of the antibodies or antigen binding fragments disclosed herein comprises the amino acid sequence of SEQ ID NO: 29, and the light chain variable region of the antibodies or antigen binding fragments disclosed herein comprises an amino acid sequence of SEQ ID NO: 80.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 30 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 81. In some embodiments, the heavy chain variable region of the antibodies or antigen binding fragments disclosed herein comprises the amino acid sequence of SEQ ID NO: 30, and the light chain variable region of the antibodies or antigen binding fragments disclosed herein comprises an amino acid sequence of SEQ ID NO: 81.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 31 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 82. In some embodiments, the heavy chain variable region of the antibodies or antigen binding fragments disclosed herein comprises the amino acid sequence of SEQ ID NO: 31, and the light chain variable region of the antibodies or antigen binding fragments disclosed herein comprises an amino acid sequence of SEQ ID NO: 82.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 32 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 83. In some embodiments, the heavy chain variable region of the antibodies or antigen binding fragments disclosed herein comprises the amino acid sequence of SEQ ID NO: 32, and the light chain variable region of the antibodies or antigen binding fragments disclosed herein comprises an amino acid sequence of SEQ ID NO: 83.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 33 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 84. In some embodiments, the heavy chain variable region of the antibodies or antigen binding fragments disclosed herein comprises the amino acid sequence of SEQ ID NO: 33, and the light chain variable region of the antibodies or antigen binding fragments disclosed herein comprises an amino acid sequence of SEQ ID NO: 84.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 34 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 85. In some embodiments, the heavy chain variable region of the antibodies or antigen binding fragments disclosed herein comprises the amino acid sequence of SEQ ID NO: 34, and the light chain variable region of the antibodies or antigen binding fragments disclosed herein comprises an amino acid sequence of SEQ ID NO: 85.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 35 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 86. In some embodiments, the heavy chain variable region of the antibodies or antigen binding fragments disclosed herein comprises the amino acid sequence of SEQ ID NO: 35, and the light chain variable region of the antibodies or antigen binding fragments disclosed herein comprises an amino acid sequence of SEQ ID NO: 86.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 36 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 87. In some embodiments, the heavy chain variable region of the antibodies or antigen binding fragments disclosed herein comprises the amino acid sequence of SEQ ID NO: 36, and the light chain variable region of the antibodies or antigen binding fragments disclosed herein comprises an amino acid sequence of SEQ ID NO: 87.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 37 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 88. In some embodiments, the heavy chain variable region of the antibodies or antigen binding fragments disclosed herein comprises the amino acid sequence of SEQ ID NO: 37, and the light chain variable region of the antibodies or antigen binding fragments disclosed herein comprises an amino acid sequence of SEQ ID NO: 88.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 38 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 89. In some embodiments, the heavy chain variable region of the antibodies or antigen binding fragments disclosed herein comprises the amino acid sequence of SEQ ID NO: 38, and the light chain variable region of the antibodies or antigen binding fragments disclosed herein comprises an amino acid sequence of SEQ ID NO: 89.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 39 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 90. In some embodiments, the heavy chain variable region of the antibodies or antigen binding fragments disclosed herein comprises the amino acid sequence of SEQ ID NO: 39, and the light chain variable region of the antibodies or antigen binding fragments disclosed herein comprises an amino acid sequence of SEQ ID NO: 90.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 40 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 91. In some embodiments, the heavy chain variable region of the antibodies or antigen binding fragments disclosed herein comprises the amino acid sequence of SEQ ID NO: 40, and the light chain variable region of the antibodies or antigen binding fragments disclosed herein comprises an amino acid sequence of SEQ ID NO: 91.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 41 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 92. In some embodiments, the heavy chain variable region of the antibodies or antigen binding fragments disclosed herein comprises the amino acid sequence of SEQ ID NO: 41, and the light chain variable region of the antibodies or antigen binding fragments disclosed herein comprises an amino acid sequence of SEQ ID NO: 92.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 42 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 93. In some embodiments, the heavy chain variable region of the antibodies or antigen binding fragments disclosed herein comprises the amino acid sequence of SEQ ID NO: 42, and the light chain variable region of the antibodies or antigen binding fragments disclosed herein comprises an amino acid sequence of SEQ ID NO: 93.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 43 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 94. In some embodiments, the heavy chain variable region of the antibodies or antigen binding fragments disclosed herein comprises the amino acid sequence of SEQ ID NO: 43, and the light chain variable region of the antibodies or antigen binding fragments disclosed herein comprises an amino acid sequence of SEQ ID NO: 94.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 44 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 95. In some embodiments, the heavy chain variable region of the antibodies or antigen binding fragments disclosed herein comprises the amino acid sequence of SEQ ID NO: 44, and the light chain variable region of the antibodies or antigen binding fragments disclosed herein comprises an amino acid sequence of SEQ ID NO: 95.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 45 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 96. In some embodiments, the heavy chain variable region of the antibodies or antigen binding fragments disclosed herein comprises the amino acid sequence of SEQ ID NO: 45, and the light chain variable region of the antibodies or antigen binding fragments disclosed herein comprises an amino acid sequence of SEQ ID NO: 96.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 46 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 97. In some embodiments, the heavy chain variable region of the antibodies or antigen binding fragments disclosed herein comprises the amino acid sequence of SEQ ID NO: 46, and the light chain variable region of the antibodies or antigen binding fragments disclosed herein comprises an amino acid sequence of SEQ ID NO: 97.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 47 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 98. In some embodiments, the heavy chain variable region of the antibodies or antigen binding fragments disclosed herein comprises the amino acid sequence of SEQ ID NO: 47, and the light chain variable region of the antibodies or antigen binding fragments disclosed herein comprises an amino acid sequence of SEQ ID NO: 98.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 48 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 99. In some embodiments, the heavy chain variable region of the antibodies or antigen binding fragments disclosed herein comprises the amino acid sequence of SEQ ID NO: 48, and the light chain variable region of the antibodies or antigen binding fragments disclosed herein comprises an amino acid sequence of SEQ ID NO: 99.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 49 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 100. In some embodiments, the heavy chain variable region of the antibodies or antigen binding fragments disclosed herein comprises the amino acid sequence of SEQ ID NO: 49, and the light chain variable region of the antibodies or antigen binding fragments disclosed herein comprises an amino acid sequence of SEQ ID NO: 100.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 50 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 101. In some embodiments, the heavy chain variable region of the antibodies or antigen binding fragments disclosed herein comprises the amino acid sequence of SEQ ID NO: 50, and the light chain variable region of the antibodies or antigen binding fragments disclosed herein comprises an amino acid sequence of SEQ ID NO: 101.

In some embodiments, the heavy chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 51 and the light chain variable region of the antibodies or antigen binding fragments disclosed herein comprises an amino acid sequence having at least about 70% (for example, about 75%, about 80%, about 85%, about 90%, about 95%, about 96%, about 97%, about 98%, about 99%, about 99.5%, or about 100%, including all subranges and values that lie therebetween) identity to SEQ ID NO: 102. In some embodiments, the heavy chain variable region of the antibodies or antigen binding fragments disclosed herein comprises the amino acid sequence of SEQ ID NO: 51, and the light chain variable region of the antibodies or antigen binding fragments disclosed herein comprises an amino acid sequence of SEQ ID NO: 102.

The disclosure further provides antibodies or antigen binding fragments thereof disclosed herein that specific bind to one or more epitopes that is bound by any one of the antibodies or antigen binding fragments thereon disclosed herein. The disclosure provides antibodies or antigen binding fragments thereof disclosed herein that compete with any one of the antibodies or antigen binding fragments thereon disclosed herein for binding to CSP-1. In some embodiments, the antibodies or antigen binding fragments thereof disclosed herein compete with an antigen binding protein described herein for binding to CSP-1.

In some embodiments, the antibody or antigen binding fragment thereof disclosed herein is a monoclonal antibody, a polyclonal antibody, a recombinant antibody, a human antibody, a humanized antibody, a chimeric antibody, a multispecific antibody, or an antibody fragment thereof. In some embodiments, the antibody or antigen binding fragment thereof disclosed herein is a Fab fragment, a Fab′ fragment, a F(ab′)₂ fragment, a Fv fragment, a single chain Fv (scFv), a diabody, a single domain antibody (sdAb), or a single chain antibody molecule.

In some embodiments, the antibody or antigen binding fragment thereof disclosed herein is a human antibody. In some embodiments, the antibody or antigen binding fragment thereof disclosed herein is a monoclonal antibody. In some embodiments, the antibody or antigen binding fragment thereof disclosed herein is coupled to a labeling group.

In some embodiments, the antibody or antigen binding fragment thereof disclosed herein is of the IgG1-, IgG2-, IgG3- or IgG4-type. In some embodiments, the antibody or antigen binding fragment thereof disclosed herein is of the IgG4- or IgG2-type. In some embodiments, the antibody or antigen binding fragment thereof disclosed herein comprises a human IgG1 or IgG4 domain. In some embodiments, the antibodies or antigen binding fragments thereof disclosed herein comprise an IgG1 domain having a constant heavy domain comprising an amino acid sequence having at least about 90% identity to SEQ ID NO: 188. In some embodiments, the antibodies or antigen binding fragments thereof disclosed herein comprise an IgG1 domain having a constant light domain comprising an amino acid sequence having at least about 90% identity to SEQ ID NO: 188.

In some embodiments, the antibodies or antigen binding fragments thereof disclosed herein comprise an IgG4 domain having a constant heavy domain comprising an amino acid sequence having at least about 90% identity to SEQ ID NO: 190. In some embodiments, the antibodies or antigen binding fragments thereof disclosed herein comprise an IgG4 domain having a constant light domain comprising an amino acid sequence having at least about 90% identity to SEQ ID NO: 190.

In some embodiments, the antibody or antigen binding fragment thereof has a binding affinity (K_{d}) for CSP-1 of about 50 nM or less. In some embodiments, the antibody or antigen binding fragment thereof has a binding affinity for CSP-1 of less than about 100 μM, for example, about 50 μM, about 25 μM, about 10 μM, about 5 μM, about 1 μM, about 500 nM, about 250 nM, about 100 nM, about 50 nM, about 10 nM, about 1 nM, about 500 μM, about 250 μM, about 100 μM, about 50 μM, about 10 μM, or about 1 μM.

In some embodiments, the binding of the antibody or antigen binding fragment thereof to CSP-1 reduces the binding of CSP-1 to one or more binding partners or ligands. The ligand or binding partner of CSP-1 is not limited, and may comprise any ligand or binding partner of plectin. Non-limiting examples of ligands or binding partners of CSP-1 include integrins (e.g., alpha 6-integrin, beta 4-integrin), extracellular matrix proteins (e.g. fibronectin and laminin), vimentin, actin, syn3, spectrin, filamin, dst, fer, tor1a, ank3, ank2, prx, bfsp1, bfsp2, ahnak, col17a1, desmin, gfap, and cytokeratins. In some embodiments, the antibody or antigen binding fragment thereof binds to a location or an epitope on CSP-1 that overlaps with the location or epitope that is bound by one or more binding partners or ligands of CSP-1. In some embodiments, the antibody or antigen binding fragment thereof competes with one or more binding partners or ligands of CSP-1 for binding to CSP-1. In some embodiments, the binding of the antibody or antigen binding fragment thereof to CSP-1 causes internalization of CSP-1 upon binding. In some embodiments, the binding of the antibody or antigen binding fragment thereof to CSP-1 induces immune-related cell death.

The disclosure further provides antibodies or antigen-binding fragments thereof that specifically bind to the epitopes disclosed herein. The disclosure also provides protein complexes, comprising the antibody or antigen-binding fragment thereof bound to CSP-1 on the surface of a cancer cell.

Fc modifications may be amino acid insertions, deletions, or substitutions, or may be chemical modifications. For example, Fc region modifications may be made to increase or decrease complement binding, to increase or decrease antibody-dependent cellular cytoxicity, or to increase or decrease the half-life of the antibody. Some Fc modifications increase or decrease the affinity of the antibody for an Fcγ receptor such as FcγRI, FcγRII, FcγRIII, or FcRn. Various Fc modifications have been described in the art, for example, in Shields et al., J Biol. Chem 276; 6591 (2001); Tai et al. Blood 119; 2074 (2012); Spiekermann et al. J Exp. Med 196; 303 (2002); Moore et al. mAbs 2:2; 181 (2010); Medzihradsky Methods in Molecular Biology 446; 293 (2008); Mannan et al. Drug Metabolism and Disposition 35; 86 (2007); and Idusogie et al. J Immunol 164; 4178 (2000). In some embodiments, Fc region glycosylation patters are altered. In embodiments, the antibody has modifications to increase or enhance effector function. In embodiments, the antibody has modifications to reduce or abolish effector function. Exemplary Fc modifications include modifications at one or more amino acid position selected from the group consisting of 228, 233, 234, 235, 236, 237, 238, 241, 248, 265, 270, 297, 309, 318, 320, 322, 331, and 409 (Kabat numbering; Kabat et al., Sequences of Immunological Interest, Fifth Edition, National Institute of Health, Bethesda, Md. (1991)). In embodiments, the antibody is an IgG1 antibody having one or more Fc modification selected from the group consisting of E233P, L234V, L234A, L235V, L235A, G236 (deleted), D265A, D270A, N297A and N297Q. In embodiments, the antibody is an IgG4 antibody having one or more Fc modification selected from the group consisting of S228P, E233P, F234A, F234V, L235A, L235V, S241P, L248E, D265A, D265T, L309L, and R409K.

The disclosure further provides compositions, comprising any of the antibodies or antigen-binding fragments thereof disclosed herein. In some embodiments, the disclosed compositions comprise any of the antibodies or antigen-binding fragments thereof disclosed herein and a heterologous moiety. In some aspects, the disclosure provides an antibody-drug conjugate comprising an anti-CSP1 antibody described herein conjugated to a heterologous moiety.

The heterologous moiety can be, e.g., a heterologous polypeptide, a therapeutic agent (e.g., a toxin or a drug), or a detectable label such as, but not limited to, a radioactive isotope, a prosthetic group, a magnetic compound, an x-ray absorber, a chemical compound, a radioactive label, an enzymatic label, a fluorescent label, a heavy metal label, a luminescent label, bioluminescent material, positron emitting metal, nonradioactive paramagnetic metal ion, or an affinity tag such as biotin or streptavidin.

Suitable heterologous polypeptides include, e.g., an antigenic tag for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa Fluor® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA).

Non-limiting examples of suitable enzymes include horseradish peroxidase, alkaline phosphatase, β-galactosidase, glucose oxidase, or acetylcholinesterase; non-limiting examples of suitable prosthetic group complexes include streptavidin/biotin and avidin/biotin; non-limiting examples of suitable fluorescent materials include biotin, umbelliferone, fluorescein, fluorescein isothiocyanate, rhodamine, dichlorotriazinylamine fluorescein, dansyl chloride, or phycoerythrin; an example of a luminescent material includes luminol; non-limiting examples of bioluminescent materials include luciferase, luciferin, and aequorin; and examples of suitable radioactive material include a radioactive metal ion, e.g., 131 125 123 121 alpha-emitters or other radioisotopes such as, for example, iodine, carbon (¹⁴C), sulfur (³⁵S), tritium (3H), indium (¹¹⁵mln, ¹¹³mln, ¹¹²In, ^{m}In) and technetium (⁹⁹Tc, ⁹⁹mTc), thallium (²⁰¹Ti), gallium (⁶⁸Ga, ⁶⁷Ga), palladium (¹¹³Pd), molybdenum (⁹⁹Mo), xenon (¹³³Xe), fluorine (¹⁸F), ¹⁵³Sm, Lu, ¹⁵⁹Gd, ¹⁴⁹Pm, ¹⁴⁰ La, ¹⁷⁵Yb, ¹⁶⁶Ho, ⁹⁰Y, ⁴⁷Sc, ⁸⁶R, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, and tin (¹¹³Sn, ¹¹⁷Sn), and radioactive labels including ³²P, ³³P, ¹⁴C, ¹²⁵I, ¹³¹I, ³⁵S and ³H.

In some embodiments, the antibodies or antigen-binding fragments thereof disclosed herein may be conjugated to a therapeutic agent, such as, a chemotherapeutic drug. Non-limiting examples of therapeutic agents include SN38, fludarabine, ibrutinib, fostamatinib, lenalidomide, thalidomide, rituximab, cyclophosphamide, doxorubicin, vincristine, prednisone, rituximab, cyclophosphamide, doxorubicin hydroxydaunomycin, vincristine, oncovin, prednisone, bendamustine, gemcitabine, oxaliplatin, cyclophosphamide, vincristine, vinblastine, anthracycline, daunorubicin, daunomycin, doxorubicin, actinomycin dactinomycin, bleomycin, clofarabine, nelarabine, cladribine, asparaginase, methotrexate, ozogamicin, monomethyl auristatin E (MMAE), Mertansine (DM1), DXd, SN-38, monomethyl auristatin E (MMAF), SG3199 or pralatrexate. In some embodiments, the therapeutic agent is a calicheamicin (e.g. ozogamicin), auristatin (e.g. MMAE, MMAF), maytansinoid (e.g., DM1), camptothecin (e.g., DXd, SN-38), or a pyrrolobenzodiazepine (PBD) dimer (e.g., SG3199).

In some embodiments, the therapeutic agent is a topoisomerase inhibitor, such as a topoisomerase I inhibitor or a topoisomerase II inhibitor. In some embodiments, the therapeutic agent is a microtubule inhibitor. In some embodiments, the therapeutic agent promotes DNA cleavage.

In some embodiments, the antibody is coupled to the heterologous moiety via a linker. As used herein, the term “linker” refers to a molecule or sequence, such as an amino acid sequence, that attaches, as in a bridge, one molecule or sequence to another molecule or sequence.

Antibodies and antigen binding fragments disclosed herein may be linked to the heterologous moiety directly, e.g., as a fusion protein with protein or peptide detectable moieties (with or without an optional linking sequence, e.g., a flexible linker sequence) or via a chemical coupling moiety. A number of such coupling moieties are known in the art, e.g., a peptide linker or a chemical linker, e.g., as described in International Patent Application Publication No. WO2009/036092. In some embodiments, the linker is a flexible amino acid sequence. Examples of flexible amino acid sequences include glycine and serine rich linkers, which comprise a stretch of two or more glycine residues. In some embodiments, the linker is a photolinker. Examples of photolinkers include ketyl-reactive benzophenone (BP), anthraquinone (AQ), nitrene-reactive nitrophenyl azide (NPA), and carbene-reactive phenyl-(trifluoromethyl)diazirine (PTD).

In some embodiments, the heterologous moiety comprises a physiologically inert nanoparticle. Examples of nanoparticles developed and used for imaging cancer cells, include magnetic nanoparticles and their magnetofluorescent analogues (see, e.g., Weissleder et al, Nat. Biotechnol., 19:316-317 (2001); McCarthy et al, Nanomedicine, 2: 153-167 (2007); Hogemann et al, Bioconjug. Chem., 11:941-946 (2000), and Josephson et al, Bioconjug. Chem., 10: 186-191 (1999)) which are contemplated for use with isolated peptide ligands and phage displayed peptides. Multimodal nanoparticles are known that incorporate both magnetic and fluorescent molecules within the same molecule and are used for fluorescent microscopy (which detects the fluorescent part of this very small particle) and MRI (which detects its magnetic portion). In some embodiments, the nanoparticle is magnetic, fluorescent, or radioactive. In some embodiments, the heterologous moiety comprises a fluorochrome.

### Pharmaceutical Compositions Comprising Anti-CSP-1 Antibodies

The disclosure also provides isolated nucleic acid molecules encoding any one of the antibodies or antigen-binding fragments thereof disclosed herein. The disclosure provides expression vectors comprising a nucleic acid segment encoding any one of the antibodies or antigen-binding fragments thereof disclosed herein. The disclosure further provides recombinant host cells comprising any one of the expression vectors.

The disclosure provides compositions comprising any one of the antibodies or antigen-binding fragments thereof disclosed herein. In some embodiments, the compositions are pharmaceutical compositions, comprising any one of the antibodies or antigen-binding fragments thereof disclosed herein and a pharmaceutically acceptable excipient.

In some embodiments, the compositions disclosed herein further comprise at least one pharmaceutically acceptable carrier, excipient, and/or vehicle, for example, solvents, buffers, solutions, dispersion media, coatings, antibacterial and antifungal agents, isotonic and absorption delaying agents. In some embodiments, the pharmaceutically acceptable carrier, excipient, and/or vehicle may comprise saline, buffered saline, dextrose, water, glycerol, sterile isotonic aqueous buffer, and combinations thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like) or suitable mixtures thereof. In some embodiments, the compositions disclosed herein further comprise minor amounts of emulsifying or wetting agents, or pH buffering agents.

In some embodiments, the compositions disclosed herein further comprise other conventional pharmaceutical ingredients, such as preservatives, or chemical stabilizers, such as chlorobutanol, potassium sorbate, sorbic acid, sulfur dioxide, propyl gallate, the parabens, ethyl vanillin, glycerin, phenol, parachlorophenol or albumin. In some embodiments, the compositions disclosed herein may further comprise antibacterial and antifungal agents, such as, parabens, chlorobutanol, phenol, sorbic acid or thimerosal; isotonic agents, such as, sugars or sodium chloride and/or agents delaying absorption, such as, aluminum monostearate and gelatin.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

In some embodiments, the compositions of the present disclosure are formulated in a neutral or salt form. Pharmaceutically-acceptable salts include, for example, acid addition salts (formed with the free amino groups of the protein) derived from inorganic acids, e.g., hydrochloric or phosphoric acids, or from organic acids, e.g., acetic, oxalic, tartaric, mandelic, and the like. In some embodiments, the salts formed with the free carboxyl groups of the protein may be derived from inorganic bases (e.g., sodium, potassium, ammonium, calcium, or ferric hydroxides) or from organic bases (e.g., isopropylamine, trimethylamine, histidine, procaine) and the like.

In some embodiments, the composition is in a solid form, such as a lyophilized powder suitable for reconstitution, a liquid solution, suspension, emulsion, tablet, pill, capsule, sustained release formulation, or powder. In some embodiments, delivery vehicles such as liposomes, nanocapsules, microparticles, microspheres, lipid particles, vesicles, and the like, may be used for the introduction of the compositions disclosed herein into suitable host cells. In some embodiments, compositions disclosed herein may be formulated for delivery either encapsulated in a lipid particle, a liposome, a vesicle, a nanosphere, or a nanoparticle or the like.

### Methods of Using Anti-CSP-1 Antibodies

The disclosure provides methods of forming a protein complex, the method comprising contacting the CSP-1 with any one of the antibodies or antigen-binding fragments thereof disclosed herein. In some embodiments, the contacting step is performed in vitro, ex vivo or in vivo. Thus, in some embodiments, the method comprises administering any one of the antibodies or antigen-binding fragments thereof disclosed herein to a subject. In some embodiments, the subject has a cancer. In some embodiments, the method comprises contacting the CSP-1 with any one of the antibodies or antigen-binding fragments thereof disclosed herein in vivo in a subject with a cancer. In some embodiments, the cancer is associated with cell surface expression of CSP-1.

The disclosure provides methods for treating a cancer comprising administering to a subject in need thereof any one of the antibodies or antigen binding fragments thereof disclosed herein, any one of the compositions disclosed herein, any one of the nucleic acid molecules disclosed herein, or any one of the expression vectors disclosed herein.

The disclosure provides methods for internalization of CSP-1 into cancer cells in a subject having cancer, the method comprising administering to the subject any one of the antibodies or antigen binding fragments thereof disclosed herein, any one of the compositions disclosed herein, any one of the nucleic acid molecules disclosed herein, or any one of the expression vectors disclosed herein.

The disclosure provides methods for inducing immune-related cell death of cancer cells in a subject having cancer, the method comprising administering to the subject any one of the antibodies or antigen binding fragments thereof disclosed herein, any one of the compositions disclosed herein, any one of the nucleic acid molecules disclosed herein, or any one of the expression vectors disclosed herein.

The disclosure provides methods for inhibiting proliferation and/or migration of cancer cells in a subject having cancer, the method comprising administering to the subject any one of the antibodies or antigen binding fragments thereof disclosed herein, any one of the compositions disclosed herein, any one of the nucleic acid molecules disclosed herein, or any one of the expression vectors disclosed herein.

In some embodiments, the cancer comprises a primary solid tumor. In some embodiments, the cancer is selected from the group consisting of breast cancer, bladder cancer, lung cancer, brain cancer, ovarian cancer, pancreatic cancer, colorectal cancer, prostate cancer, liver cancer, hepatocellular carcinoma, kidney cancer, stomach cancer, skin cancer, fibroid cancer, lymphoma, virus-induced cancer, oropharyngeal cancer, testicular cancer, thymus cancer, thyroid cancer, melanoma, and bone cancer.

In some embodiments, the breast cancer comprises ductal carcinoma, lobular carcinoma, medullary carcinoma, colloid carcinoma, tubular carcinoma, or inflammatory breast cancer. In some embodiments, the breast cancer comprises ductal carcinoma. In some embodiments, the breast cancer comprises lobular carcinoma. In some embodiments, the breast cancer comprises medullary carcinoma. In some embodiments, the breast cancer comprises colloid carcinoma. In some embodiments, the breast cancer comprises tubular carcinoma. In some embodiments, the breast cancer comprises inflammatory breast cancer. In some embodiments, the breast cancer is triple-negative breast cancer. In some embodiments, the breast cancer does not respond to hormonal therapy or therapeutics that target the HER2 protein receptors.

In some embodiments, the cancer is selected from the group consisting of pancreatic cancer, ovarian cancer, gastric cancer, colorectal carcinoma, cholangiocarcinoma, head and neck cancer (such as, for example, head and neck squamous cell carcinoma (HNSCC)), triple negative breast cancer (TNBR), prostate cancer, and lung cancer (such as, non-small cell lung cancer (NSCLC) and small cell lung cancer (SCLC)). In some embodiments, pancreatic cancer is exocrine pancreatic cancer (such as, for example, adenocarcinoma, squamous cell carcinoma, adenosquamous carcinoma, and colloid carcinoma) or neuroendocrine pancreatic cancer. In some embodiments, the ovarian cancer is epithelial tumor, stromal tumor or germ cell tumor. In some embodiments, the gastric cancer is adenocarcinoma, primary gastric lymphoma, gastrointestinal stromal tumor (GIST), or neuroendocrine (carcinoid) tumors in the stomach. In some embodiments, the colorectal cancer is adenocarcinoma, gastrointestinal stromal tumors (GIST), lymphoma, carcinoids, turcot syndrome, Peutz-Jeghers syndrome (PJS), familial colorectal cancer (FCC), or juvenile polyposis coli. In some embodiments, head and neck cancer is laryngeal cancer, hypopharyngeal cancer, nasal cavity and paranasal sinus cancer, nasopharyngeal cancer, oral cancer, oropharyngeal cancer, or salivary gland cancer.

In some embodiments, the immune-related cell death induced by the administration of the antibody or antigen binding fragment thereof increases anti-cancer T cell responses in the subject. Anti-cancer T cell activity may be evaluated by observing the induction of pyroptosis (via Caspase-1 cleavage, gasedermin cleavage, secretion of cytokines), induction of stress pathways, immune subset analysis, and/or co-culture studies.

In some embodiments, the administration of the antibody or antigen binding fragment thereof elicits an increase in effector memory T cells in the subject. In some embodiments, the administration of the antibody or antigen binding fragment thereof elicits at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, or about 10 fold, including all values and subranges that lie therebetween) increase in effector memory T cells in the subject. In some embodiments, the administration of the antibody or antigen binding fragment thereof elicits at least a 2 fold increase in effector memory T cells in the subject.

In some embodiments, the administration of the antibody or antigen binding fragment thereof increases macrophage infiltration of tumors in the subject. Macrophage infiltration may be evaluated using immune subset analysis and immunohistochemistry (IHC) for F480.

In some embodiments, the methods disclosed herein may comprise administering to the subject a therapeutically effective amount of any one of the antibodies or antigen binding fragments thereof, any one of the compositions, any one of the nucleic acid molecules, or any one of the expression vectors disclosed herein in combination with one or more secondary therapies targeting cancer. In some embodiments, the methods of treating cancer in a subject disclosed herein may further comprise administering one or more secondary therapies targeting cancer. In some embodiments, the secondary therapy comprises chemotherapy, immunotherapy, targeted therapy, an immune checkpoint inhibitor, radiation therapy, hormone therapy, phototherapy, virotherapy, or any combination thereof. In some embodiments, the chemotherapeutic agent is fludarabine, ibrutinib, fostamatinib, lenalidomide, thalidomide, rituximab, cyclophosphamide, doxorubicin, vincristine, prednisone, rituximab, cyclophosphamide, doxorubicin hydroxydaunomycin, vincristine, oncovin, prednisone, or any combination thereof.

The term administered “in combination,” as used herein, is understood to mean that two (or more) different treatments are delivered to the subject during the course of the subject's affliction with the disorder (such as, cancer), such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent” delivery. In other embodiments, the delivery of one treatment ends before the delivery of the other treatment begins, which may be referred to as “sequential” delivery.

In some embodiments, the treatment is more effective because of combined administration. For example, the second treatment is more effective; for e.g., an equivalent effect is seen with less of the second treatment, or the second treatment reduces symptoms to a greater extent, than would be seen if the second treatment were administered in the absence of the first treatment, or the analogous situation is seen with the first treatment. The effect of the two treatments can be partially additive, wholly additive, or greater than additive (synergistic).

### EXAMPLES

It is to be understood that the description above as well as the examples that follow are intended to illustrate, and not limit, the scope of the invention. Other aspects, advantages and modifications within the scope of the invention will be apparent to those skilled in the art to which the invention pertains.

### Example 1: Humanization of Mouse Anti-CSP-1 Antibodies

The murine anti-CSP-1 hybridoma clone, 1D7, was used to make humanized versions of anti-CSP-1 antibodies. The heavy chain variable region of 1D7 comprises the amino acid sequence of SEQ ID NO: 137, and the light chain variable region of 1D7 comprises the amino acid sequence of SEQ ID NO: 138. The HCDR1, HCDR2, and HCDR3 of 1D7 comprise the amino acid sequence of SEQ ID NO: 104, 115, and 125, respectively. The LCDR1, LCDR2, and LCDR3 of 1D7 comprise the amino acid sequences of SEQ ID Nos 107, 120, and 133, respectively.

Humanization was performed in silico using the human-engineering (HE) method. The constant domains were replaced with the homologous domains from human IgG1. The surface of the VH and VL domain were humanized by substituting human framework residues in positions that are known to be solvent exposed in the immunoglobulin fold. A total of 17 variants were synthesized to explore different combinations of human vs. murine sequences. These variants were purified by Protein A affinity chromatography and tested for binding to recombinant CSP to determine if they possessed similar binding affinity and specificity to CSP as muZB131. The lead candidate, huZB131, was chosen due to its equivalence to muZB131 for binding to CSP. This sequence also eliminates a N-linked glycosylation site, two potential asparagine deamidation sites, and one site for potential methionine oxidation. Further details on the humanization method is provided in Studnicka, G. M., Soares, S., Better, M., Williams, R. E., Nadell, R., and Horwitz, A. H. (1994) Human-engineered monoclonal antibodies retain full specific binding activity by preserving non-CDR complementarity-modulating residues. Protein Eng. 7, 805-814, which is incorporated herein by reference in its entirety for all purposes.

After humanization, the antibodies were tested for their capability to bind to an antigenic region of CSP-1 called section 8 (Sec8). Antigen binding was assessed by single-shot Octet analysis. Antibodies were loaded onto anti-human IgG tips, and the loaded tips were dipped into wells containing the Sec8-His antigen. Scouting experiments were used to determine ideal conditions for loading (10 minute incubation in 1 μg/mL IgG) and antigen binding (5 minute incubation in 24 nM Sec8-His, followed by a 5 minute dissociation step in buffer). Two negative controls were included with each experiment. First, a tip was loaded with irrelevant IgG dipped into 24 nM Sec8-His to control for non-specific interactions between the tip and the antigen. Next, tips loaded with humanized 1D7 variants were dipped into 24 nM GST-His to control for off-target binding of the antibody (a significant risk if a given variant is destabilized and/or exposes significant hydrophobic surface). The data from the irrelevant IgG control were subtracted from the data obtained with the anti-plectin-loaded tips. The mean and standard deviation of the signal change values for the GST-His control wells were used to calculate a threshold for positive binding (mean+3 standard deviations of the GST-His signal). Variants that were below this threshold are considered to have no specific binding to Sec8. Humanized anti-CSP1 antibodies that showed high affinity to CSP-1 were tested further to evaluate their ability to suppress OVCAR8 and MiaPaca-2 cell proliferation, and/or suppress the growth of tumors in vivo.

The SEQ ID Nos of the heavy chain variable region (VH) sequences and the light chain variable region (VL) sequences of the humanized antibodies that were generated using the methods described above and that showed successful binding to CSP-1 are listed in Table 1. In addition, the SEQ ID Nos of the three complementarity determining region (CDR) sequences of the VH and VL regions are also listed in Table 1.

### Example 2: Humanized Anti-CSP-1 Antibody, Antibody 51 (Ab51), Specifically Binds to CSP-1

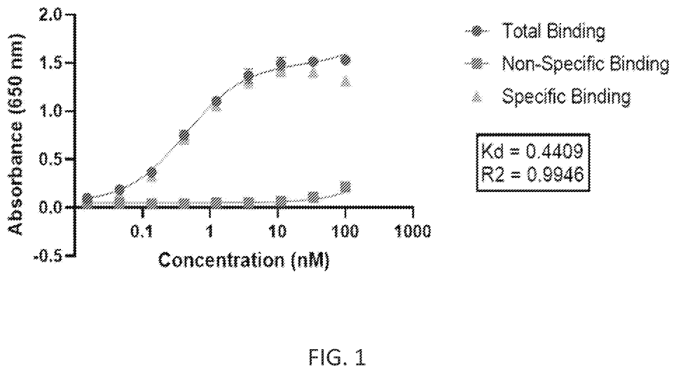

The binding of humanized anti-CSP-1 antibody (Ab51) to an antigenic region of CSP-1 called section 8 (Sec8) was measured using ELISA. As shown in FIG. 1, Ab51 shows specific binding to Sec8-His (Sec8 tagged with a polyhistidine (His) tag, comprising the amino acid sequence of SEQ ID NO: 142), and minimal non-specific binding to GST-His. The Kd of binding was calculated to be 0.4409 nM.

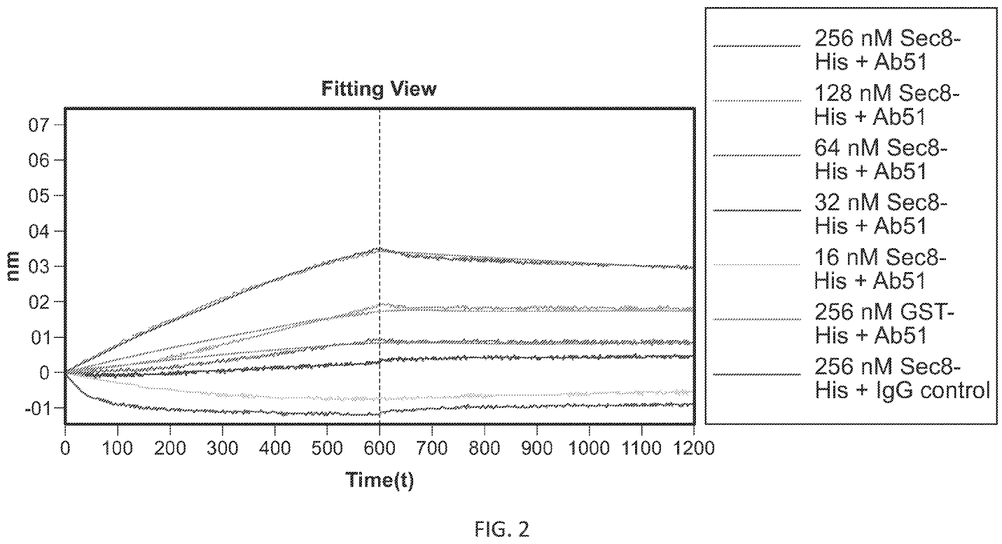

Binding of Ab51 to Sec8-His was further investigated using bio-layer interferometry, as shown in FIG. 2. Antibodies were loaded onto anti-human IgG tips (10 minutes incubation with 1 μg/mL Ab51), and the loaded tips were dipped into wells containing the Sec8-His antigen at the indicated concentrations. Antigen association was monitored for 600 seconds. The tips were moved to wells containing buffer, and dissociation was monitored for 600 seconds. The data were fit to a 1:1 model to determine on and off rates.

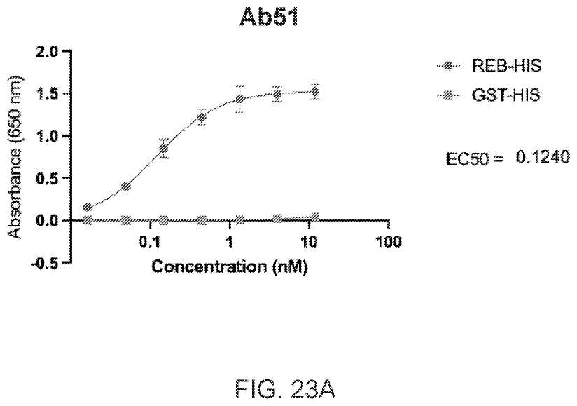

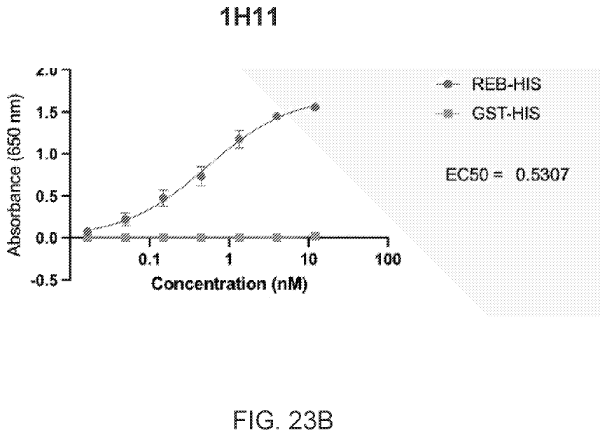

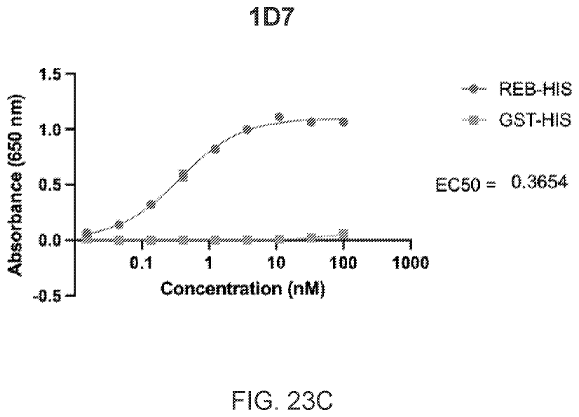

Furthermore, the binding of Ab51 to a sub-fragment of Sec8, called Reb, was measured using ELISA. As shown in FIG. 23A, Ab51 shows specific binding to Reb-His (Reb tagged with a polyhistidine (His) tag, comprising the amino acid sequence of SEQ ID NO: 395, and minimal non-specific binding to GST-His. Notably, the binding characteristics of Ab51 was comparable to the binding of the murine anti-CSP-1 antibodies 1H11 (FIG. 23B) and 1D7 (FIG. 23C), indicating that the murine antibodies serve as a surrogate in studying the functional properties of the humanized anti-CSP antibodies disclosed herein, such as Ab51.

### Example 3: Administration of a Humanized Anti-CSP-1 Antibody Suppresses the Growth of Ovarian Cancer Cells In Vivo

To test whether humanized anti-CSP-1 antibodies (such as, Ab51) have therapeutic effects on cancer, the following experiment was performed.

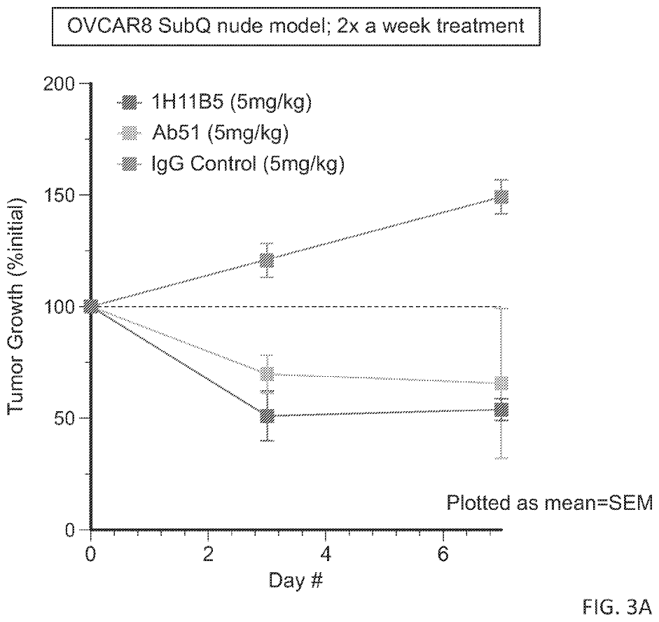

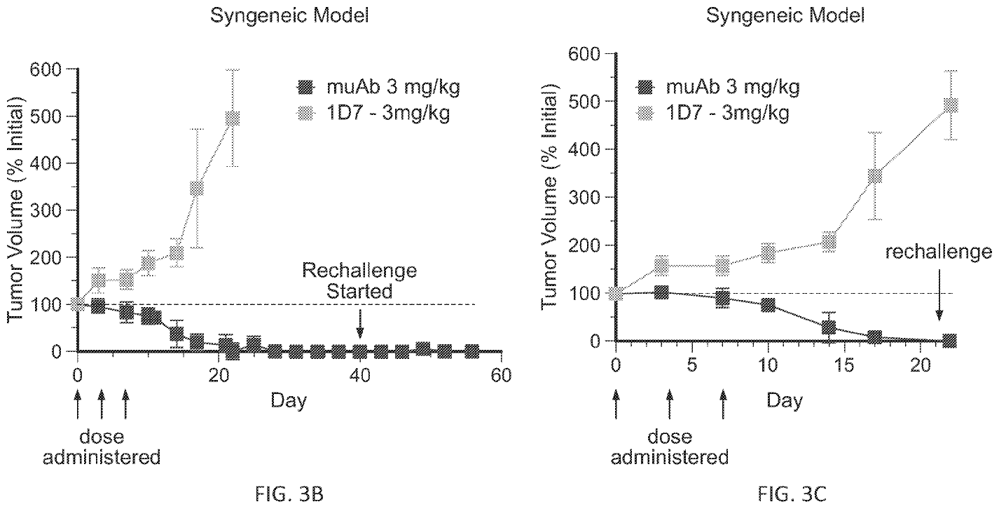

The ovarian cancer cell line (OVCAR8) was implanted into nude mice using subcutaneous injections, and tumors were allowed to form. Mice with OVCAR8 tumors were administered 5 mg/kg of either Ab51 or the control IgG antibody twice a week, and tumor growth was measured over time. As shown in FIG. 3A, the tumor volume decreased over time in mice, which were administered Ab51, as compared to control mice, which were administered IgG antibody.

These results demonstrate that administration of humanized anti-CSP-1 antibodies (such as, Ab51) suppresses the growth of tumors from ovarian cancer cells implanted into murine models.

### Example 4: Treatment with Anti-CSP-1 Antibody Results in Tumor Regression Via Immune-Related Tumor Cell Death

To further investigate mechanisms by which anti-CSP-1 antibodies inhibit tumor growth, the following experiments were performed, which demonstrated that administration of anti-CSP-1 antibodies can activate the immune system to drive tumor regression by promoting immune-related tumor cell death.

Syngeneic KPC915 pancreatic tumor cells were implanted into immunocompetent mice to generate murine models of pancreatic cancer. The mice were administered 3 mg/kg of either murine anti-CSP-1 antibody (referred to herein as “muAb”) or control IgG antibody. As shown in FIGS. 3B and 3C, while administration of control IgG antibody did not influence tumor growth, strikingly, administration of murine anti-CSP-1 antibody was able to drive tumor regression.

Without being bound to a theory, it is thought that the tumor regression in these immunocompetent mice is driven by immune-related tumor cell death. In particular, it is thought that the binding of the anti-CSP-1 antibodies disclosed herein to CSP-1 on tumor cell membrane can result in reactive oxygen species (ROS) accumulation and thereby, the formation of the inflammasome complex. The inflammasome complex causes cleavage of Caspase 1 and Gasdermin C, which in turn results in pores in the tumor cell membrane. Cytokines, such as, IL-1β and IL-18, are released from these pores and attract T-cells and macrophages to the tumor site. Tumor cells are destroyed by this localized anti-tumor immune response resulting in tumor regression.

To confirm that administration of anti-CSP-1 antibody results in tumor regression via immune-related tumor cell death, the following experiments were performed. The results described below show that anti-CSP-1 antibodies are capable of promoting each step of the signaling pathway that leads to immune-related tumor cell death.

Treatment with Anti-CSP-1 Antibodies Upregulates Stress Pathways

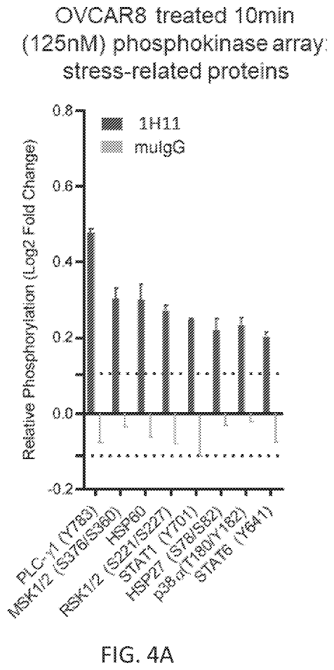

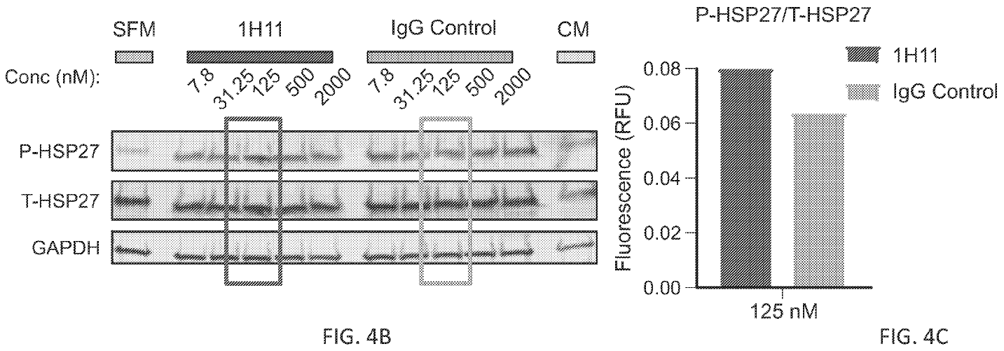

To investigate whether anti-CSP-1 antibodies can promote the immune-related cell death of tumor cells, it was evaluated whether treatment of cancer cells with anti-CSP-1 antibodies can upregulate stress pathways. As shown in FIG. 4A, OVCAR8 cells treated for 10 mins with 125 nM of a murine anti-CSP-1 antibody showed elevated phosphorylation levels of stress-related proteins, such as PLC-γ1 and MSK1/2, relative to a comparable treatment with a control IgG antibody. Additionally, FIGS. 4B and 4C show the elevated ratio of phosphorylated HSP27 to unphosphorylated HSP-27 in OVCAR8 cells after treatment with 125 nM murine anti-CSP-1 antibody, as compared to a control IgG antibody. These results show that treatment with anti-CSP-1 antibodies can increase the phosphorylation of stress-related proteins, which can lead to the induction of immune-related tumor cell death.

Treatment with Anti-CSP-1 Antibodies Increases ROS Accumulation

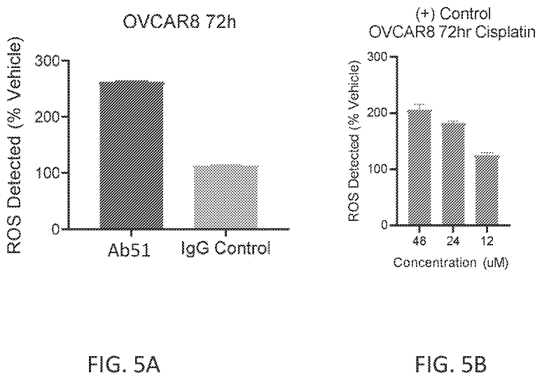

To investigate whether anti-CSP-1 antibodies can promote the production of reactive oxygen species (ROS), the amount of ROS accumulated within ovarian cancer OVCAR8 cells was measured after treatment with a 2 uM of control IgG antibody or a murine anti-CSP-1 antibody for 72 hours. As shown in FIG. 5A, the amount of ROS was higher in cells treated with the murine anti-CSP-1 antibody, as compared to the control antibody. As a positive control, OVCAR8 cells were treated for 72 hours with different concentrations of cisplatin, a chemotherapeutic drug. As shown in FIG. 5B, the amount of ROS accumulated was seen to increase in a manner dependent on the concentration of cisplatin used to treat the cells. Furthermore, ROS accumulation was also observed in anti-CSP-1-treated cholangiocarinoma and pancreatic cancer cell lines (such as, WITT, HuH28, HuCCT1, KPC915 and other KPC murine model derived cell lines). Overall, these results show that anti-CSP-1 antibodies can promote the production of reactive oxygen species (ROS) in different types of cancer cells leading to inflammasome complex formation and the induction of immune-related tumor cell death.

Treatment with Anti-CSP-1 Antibodies Promotes Cleavage of Caspase 1 and Gasdermin

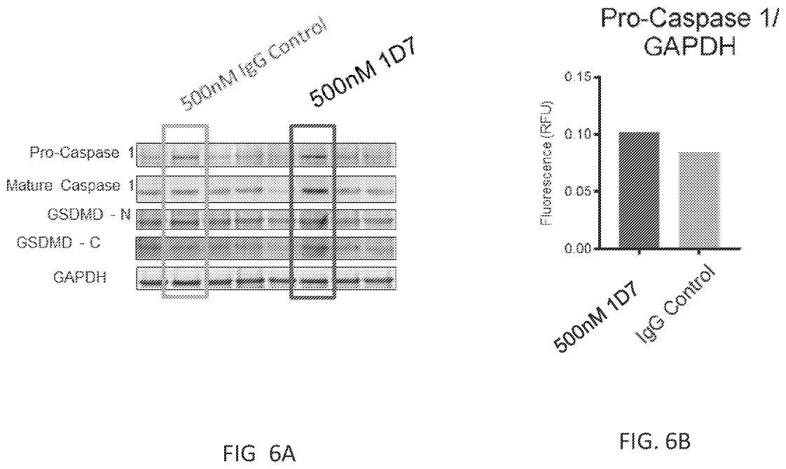

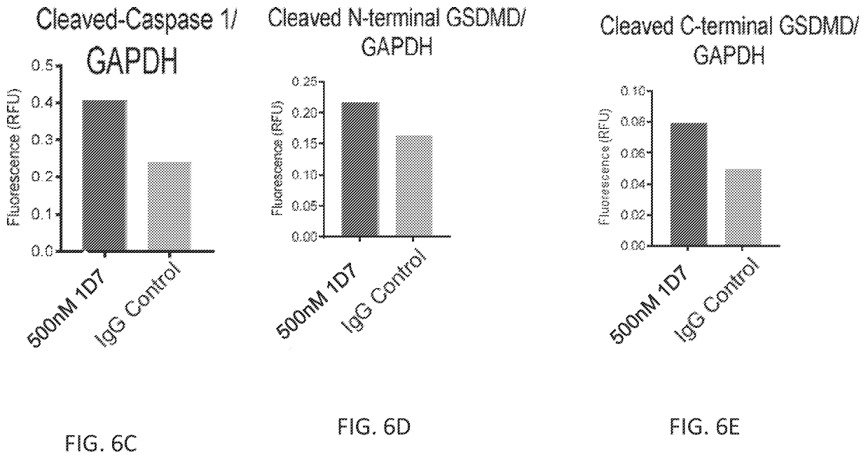

To investigate whether anti-CSP-1 antibodies can promote the cleavage of caspase-1 and gasdermin, the murine pancreatic cancer cell line KPC915 was treated for 24 hours with 500 nM of either control IgG antibody, or a murine anti-CSP-1 antibody and the levels of uncleaved pro-caspase 1, cleaved caspase 1, cleaved N-terminal region of gasdermin D (GSDMD) and cleaved C-terminal region of gasdermin D was measured relative to the housekeeping protein GAPDH using Western Blot, as shown in FIG. 6A, and as quantitated in FIGS. 6B-6E. The results show that treatment of cancer cells with anti-CSP-1 antibodies increases the cleavage of caspase 1 and gasdermin by the inflammasome complex, resulting in the opening of tumor cell membrane pores and cytokine secretion, thereby resulting in immune-related tumor cell death.

Treatment with Anti-CSP-1 Antibodies Promotes Cytokine Secretion

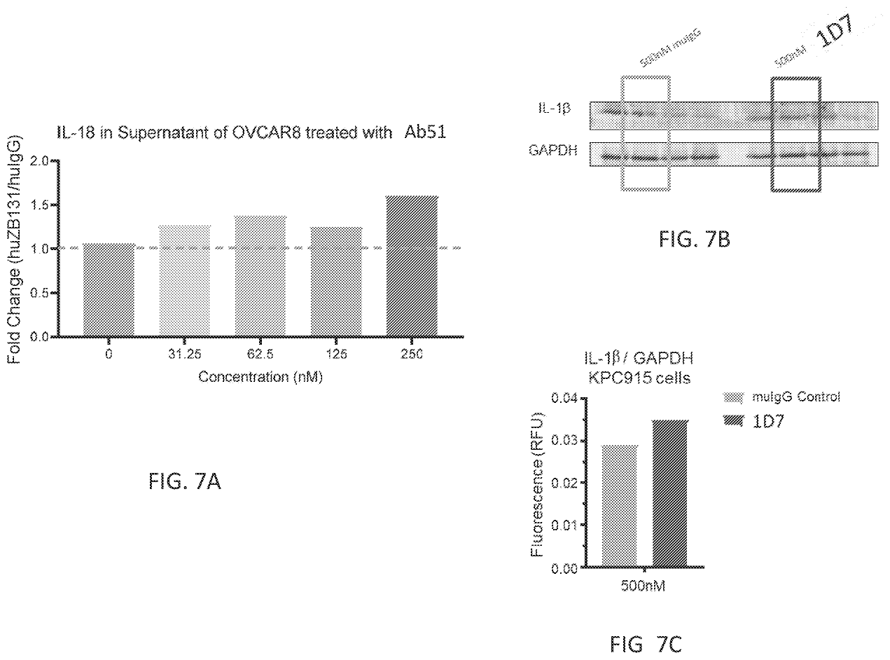

To investigate whether anti-CSP-1 antibodies can promote the secretion of cytokines, such as IL-18, OVCAR8 cells were treated for 24 hours with increasing concentrations of Ab51 or the control antibody. The increase in IL-18 secretion in the supernatant of Ab51-treated OVCAR8 cells, relative to control antibody-treated cells is shown in FIG. 7A. Murine pancreatic cancer KPC915 cells were also treated with 500 nM of murine anti-CSP-1 antibody or the control antibody, and the level of secreted IL-18 was measured using Western Blot as shown in FIG. 7B, and as quantitated in FIG. 7C. These results show that treatment of cancer cells with anti-CSP-1 antibodies results in secretion of cytokines, which induces immune-related tumor cell death.

The results described above indicate that the treatment of cancer cells with anti-CSP-1 antibodies results in induction of the stress pathways and ROS accumulation, which in turn results in cleavage of caspase 1 and gasdermin, leading to cytokine secretion. Without bound by a theory, it is thought that cytokine secretion can attract T-cells and macrophages to the tumor, resulting in the immune-related tumor cell death.

Treatment with Anti-CSP-1 Antibodies Attracts T-Cells to the Tumor Site

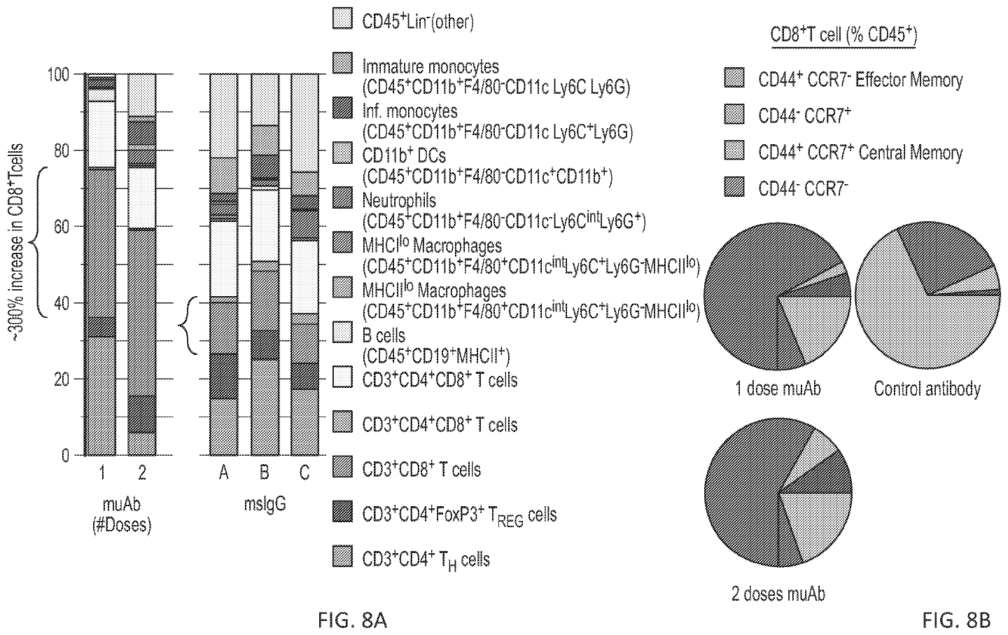

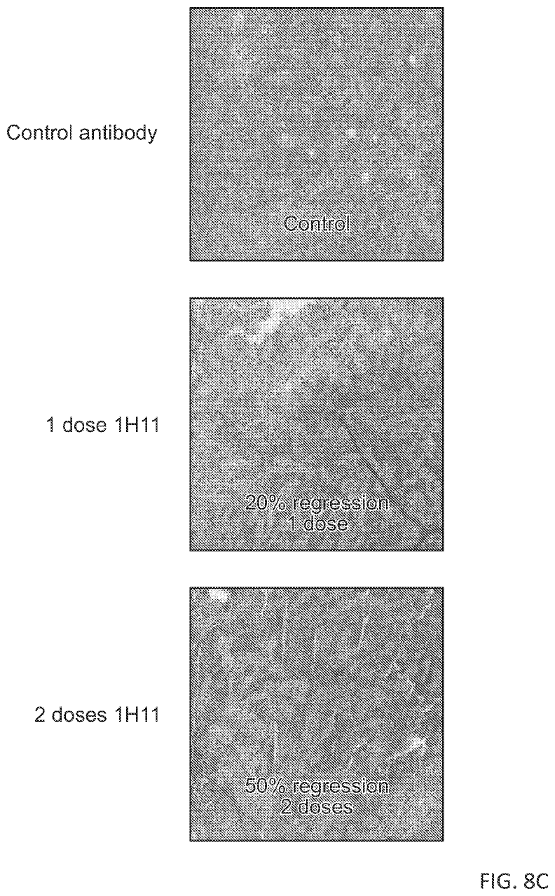

To investigate whether administration of anti-CSP-1 antibodies can promote the migration of T-cells to the tumor site, the following experiment was done. Murine models of KPC915-derived pancreatic ductal adenocarcinoma (PDAC) tumors were treated with one or two doses of murine anti-CSP-1 antibody, or a control IgG antibody. After treatment, the subset of immune cells at the tumor site was analyzed. Strikingly, as shown in FIG. 8A, there was about 300% increase in the proportion of CD8+ T cells in the tumors isolated from mice treated with the murine anti-CSP-1 antibody, as compared to the control antibody. Furthermore, it was seen that among CD8+ T cells, the percentage of CD44⁺ CCR7⁻ Effector Memory T-cells was dramatically increased by at least 2-fold, as compared to other subtypes of CD8+ T cells (FIG. 8B). Additionally, histological analysis of the PDAC tumor tissue from mice treated with one or two doses of murine anti-CSP-1 antibody, or a control IgG antibody shows that there is significant lymphocyte infiltration in anti-CSP-1 antibody-treated mice tumors (FIG. 8C). These results demonstrate that treatment with anti-CSP-1 antibodies can initiate a T-cell response against the tumors, leading to immune-related tumor cell death and thereby, tumor regression.

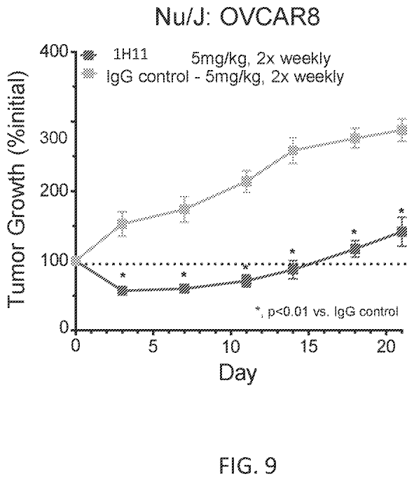

To further highlight the role played by T-cells in promoting tumor regression, the following experiment was performed using a nu/J murine model that lacks T-cells and has a partial defect in B-cells. These mice were implanted with OVCAR8 ovarian cancer cells and treated with 5 mg/kg anti-CSP-1 antibody or control IgG antibody twice a week. Tumor growth was measured over time. As shown in FIG. 9, the tumor growth in anti-CSP-1 antibody-treated mice did not completely regress, but rather halted for a period of time, and considerably slowed down. These results further support that treatment with anti-CSP-1 antibodies causes tumor regression by attracting T-cells to the tumor site, which then results in immune-related tumor cell death.

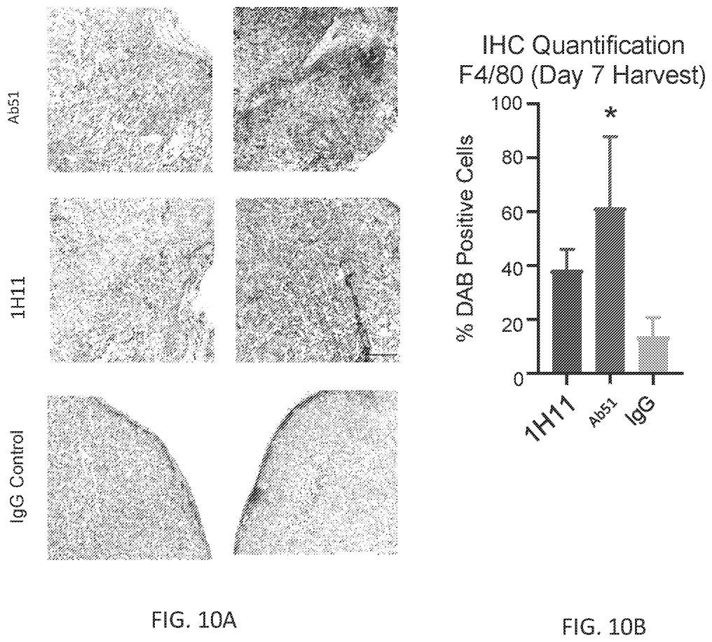

Furthermore, tumor tissue from these mice was also analyzed for the presence of macrophages. Immunohistochemistry using marker F4/80, a glycoprotein expressed by murine macrophages showed that there was a higher percentage of 3,3′-diaminobenzidine (DAB)-positive cells in tumor tissue from Ab51-treated or murine anti-CSP-1 antibody-treated mice, as compared to control antibody-treated mice (see images in FIG. 10A, and quantitation in FIG. 10B). The proportion of macrophages was also particularly higher in Ab51-treated mice, indicating that there is a significant accumulation of macrophages in OVCAR8 tumors in the absence of T-cells.

### Example 5: Treatment with Anti-CSP-1 Antibodies Attracts Myeloid Cells to the Tumor Site

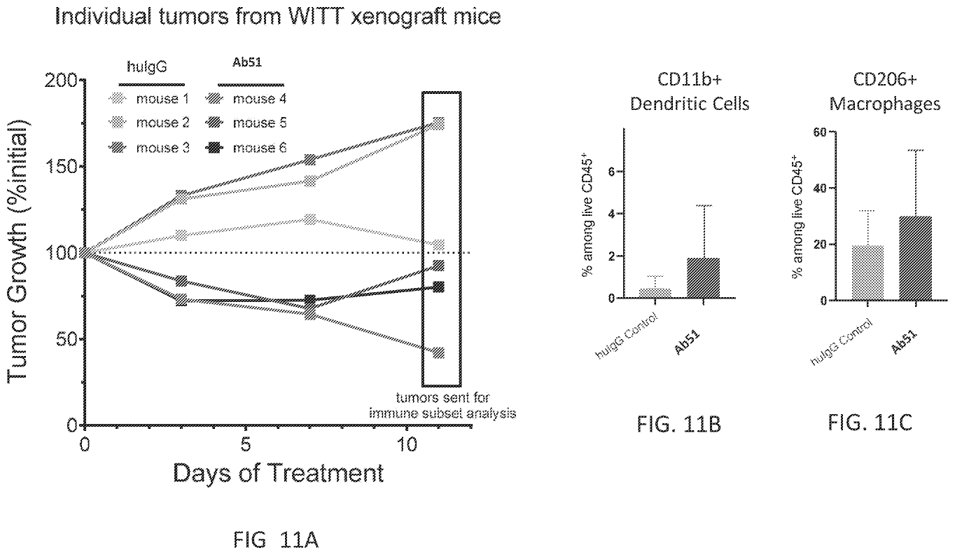

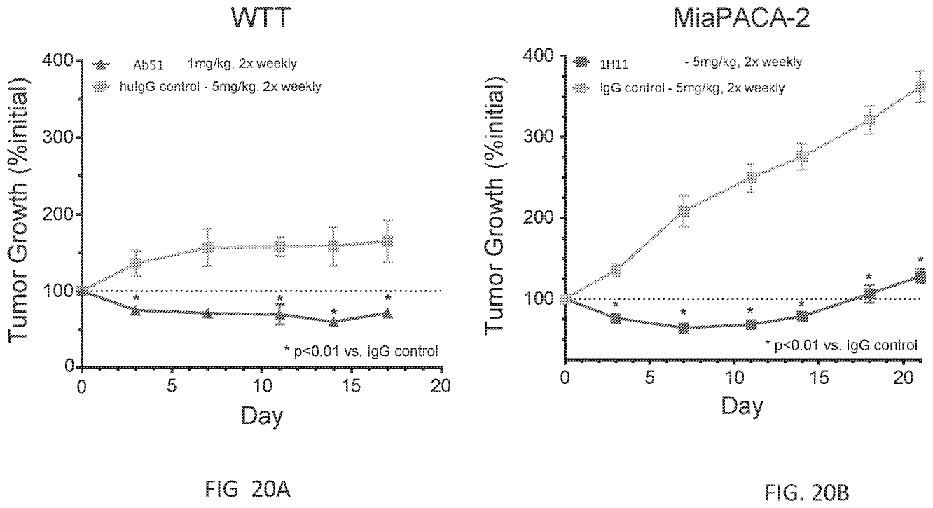

To investigate whether administration of anti-CSP-1 antibodies can promote the migration of dendritic cells and macrophages to the tumor site, extrahepatic cholangiocarcinoma (ECC) WITT cells were implanted into mice to generate a cholangiocarcinoma xenograft mouse model. These mice were treated with either 5 mg/kg control IgG antibody or 1 mg/kg Ab51 twice a week, and tumor growth was measured over time. As shown in FIG. 11A and FIG. 20A, the tumor growth decreased over time in mice, which were administered Ab51, as compared to control mice, which were administered IgG antibody. These results show that the anti-tumor effects of humanized anti-CSP-1 antibodies extend to other cancer types.

Tumors from the IgG-treated mice or Ab51-treated mice were isolated and analyzed for the presence of myeloid cells, such as, dendritic cells and macrophages. As shown in FIGS. 11B and 11C, the proportion of dendritic cells and macrophages in tumors isolated from Ab51-treated mice was higher than the proportion of dendritic cells and macrophages in tumors isolated from IgG-treated mice. As described above, without being bound by a theory, it is thought that the increased presence of myeloid cells in the tumor microenvironment is associated with the induction of immune-related tumor cell death leading to the regression of tumor growth in Ab51-treated mice.

### Example 6: Treatment with Humanized Anti-CSP-1 Antibody Arrests the Proliferation of Cancer Cells

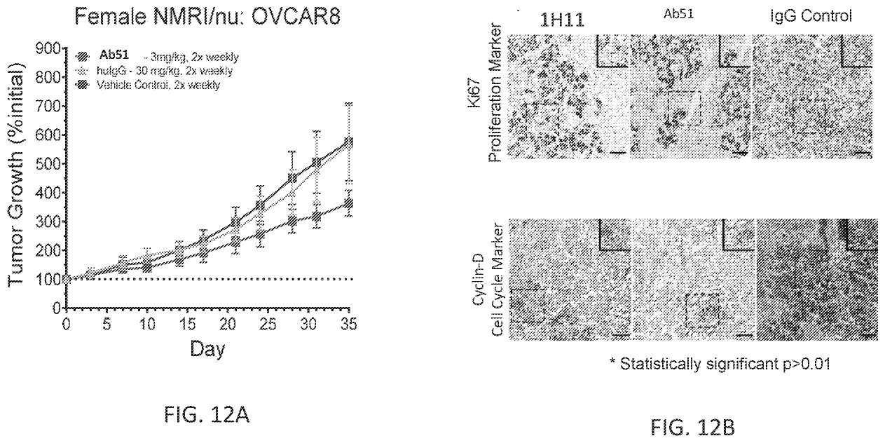

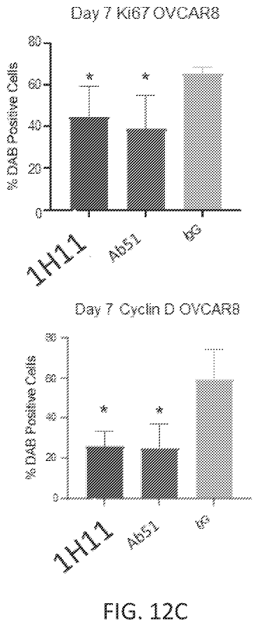

The ovarian cancer cell line (OVCAR8) was implanted into a female NMRI/nu murine model that lacks T-cells, has partially defective B-cells, and reduced monocytes/macrophages using subcutaneous injections, and tumors were allowed to form. Mice with OVCAR8 tumors were administered 3 mg/kg of Ab51 or 30 mg/kg control IgG antibody twice a week, and tumor growth was measured over time. As shown in FIG. 12A, tumors did not completely regress, but tumor growth was slower in Ab51-treated mice, as compared to control antibody-treated mice. Similar results were seen with the implantation of extrahepatic cholangiocarcinoma (ECC) WITT cells (FIG. 20A) and MiaPACA-2 cells (FIG. 20B). As described in Example 4, anti-CSP-1 antibodies are thought to promote tumor regression by inducing immune-related tumor cell death via the action of T-cells and macrophages. Without being bound by a theory, it is thought that since these mice lack T-cells and have reduced macrophages, tumor regression is not seen upon administration of anti-CSP-1 antibodies. However, tumor growth arrest is still observed in these mice lacking an effective immune system, suggesting that anti-CSP-1 antibodies likely have other direct effects on tumor growth independent of the immune system.

To investigate whether anti-CSP-1 antibodies affect tumor cell proliferation and thereby lead to tumor growth arrest, the levels of protein markers of cell proliferation and cell cycle, such as, Ki67 and cyclin D were evaluated in tumors isolated from murine OVCAR8 xenograft models. The results show that Ki67 and cyclin D were reduced in mice treated with Ab51 or murine anti-CSP1 antibody, as compared to mice treated with the control IgG antibody (FIGS. 12B and 12C).

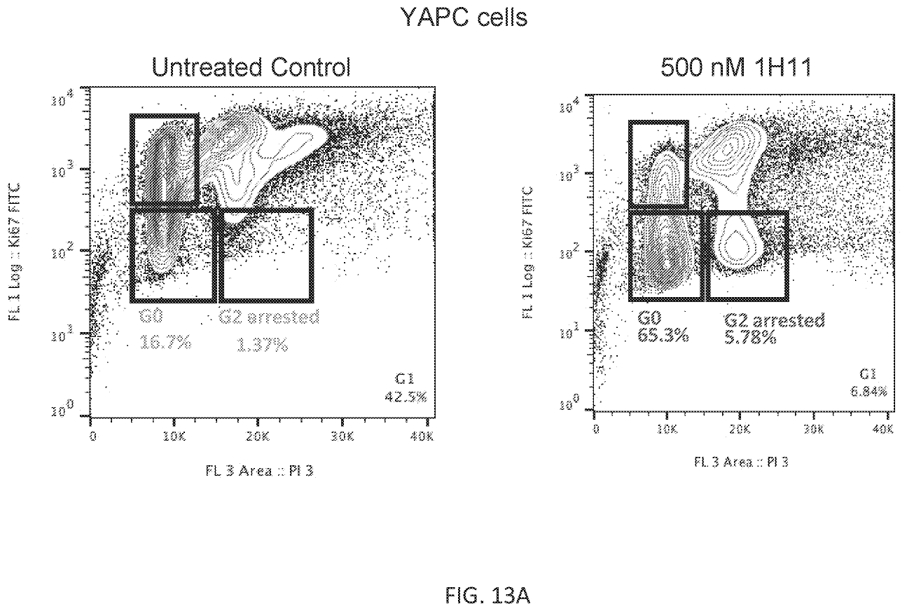

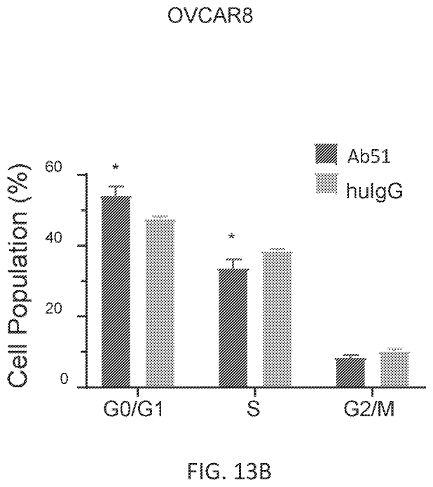

Furthermore, analysis of untreated or Ab51-treated OVCAR8 cells using flow cytometry showed that while only 16.7% of untreated cancer cells were in G0 stage of the cell cycle, more than 65% of Ab51-treated cancer cells were in G0 stage (FIG. 13A). These results indicate that the majority of Ab51-treated cancer cells were arrested in the growth (G0) phase of the cell cycle, and were not proliferating. Treatment of ovarian cancer cell line OVCAR8 with Ab51 for 72 hours also resulted in an increase in the proportion of growth phase (G0/G1)-arrested cells, as compared to control IgG-treated cells, as shown in FIG. 13B. Treatment of cholangiocarinoma and pancreatic cancer cell lines (such as, WITT, HuH28, HuCCT1, and MIA PaCa-2) also resulted in the arrest of cells in the growth phase.

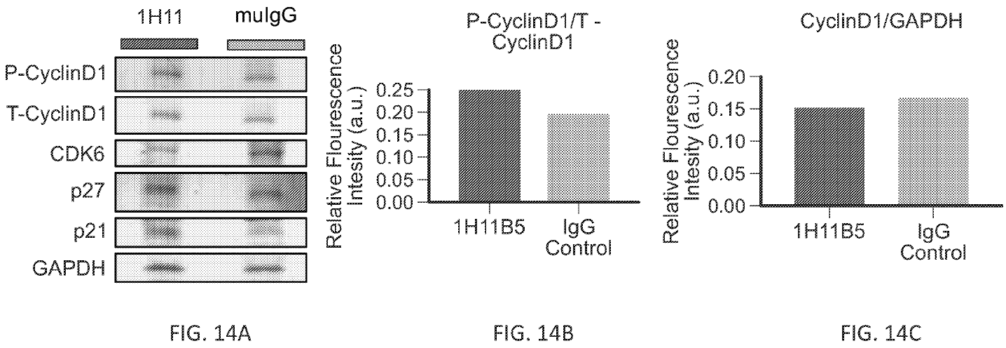

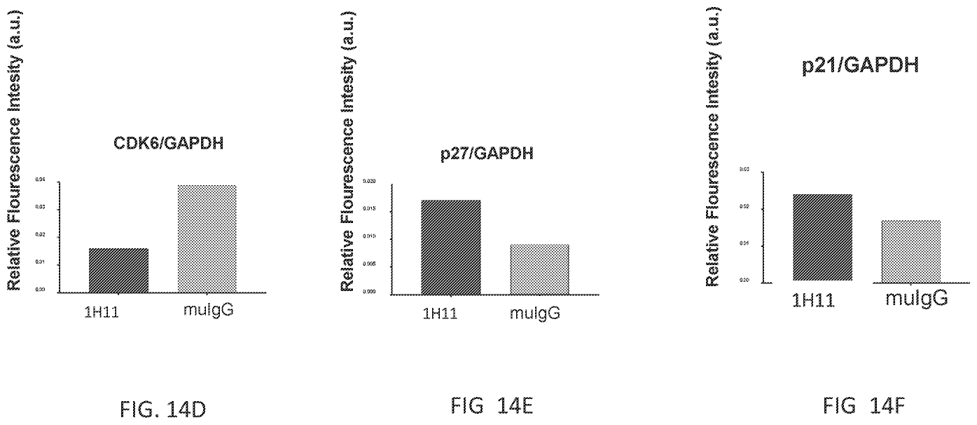

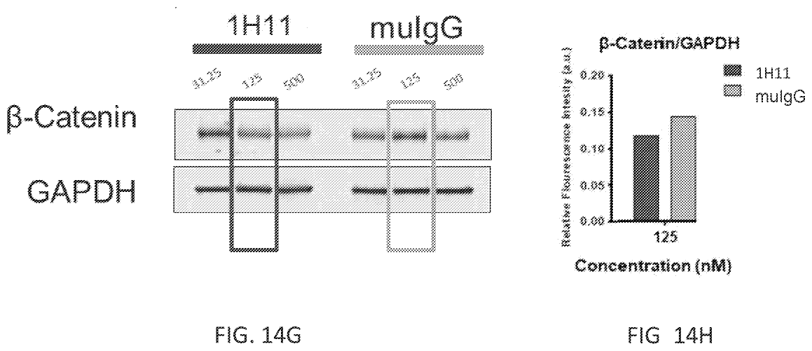

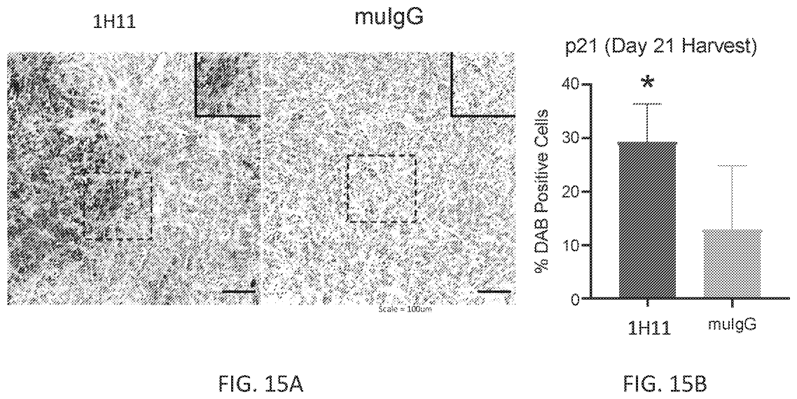

To further investigate the anti-proliferative effects of anti-CSP-1 antibodies, cells were treated with 125 nM murine anti-CSP-1 antibody, or control IgG antibody, and the levels of signaling proteins involved in proliferation such as cyclin D1 (phosphorylated and unphosphorylated forms), CDK6, p27 and p21, and the level of a signaling protein involved in migration (0-catenin) were analyzed using Western Blot. As shown in FIGS. 14A-14C, FIG. 14E and FIG. 14F, the levels of phosphorylated cyclin D1, p27 and p21 were increased relative to the housekeeping GAPDH protein upon treatment with murine anti-CSP-1 antibody, as compared to treatment with control antibody. Also, the levels of CDK6 and β-catenin were decreased relative to the housekeeping GAPDH protein upon treatment with murine anti-CSP-1 antibody, as compared to treatment with control antibody (FIGS. 14D, 14G, and 14H). Upregulation of p21 levels was also seen in vivo in tumor tissues isolated from mice treated with either the murine anti-CSP-1 antibody or the control IgG antibody (FIGS. 15A and 15B). These results demonstrate that treatment with anti-CSP-1 antibodies promotes ROS-induced anti-proliferative and anti-migratory effects on cancer cells.

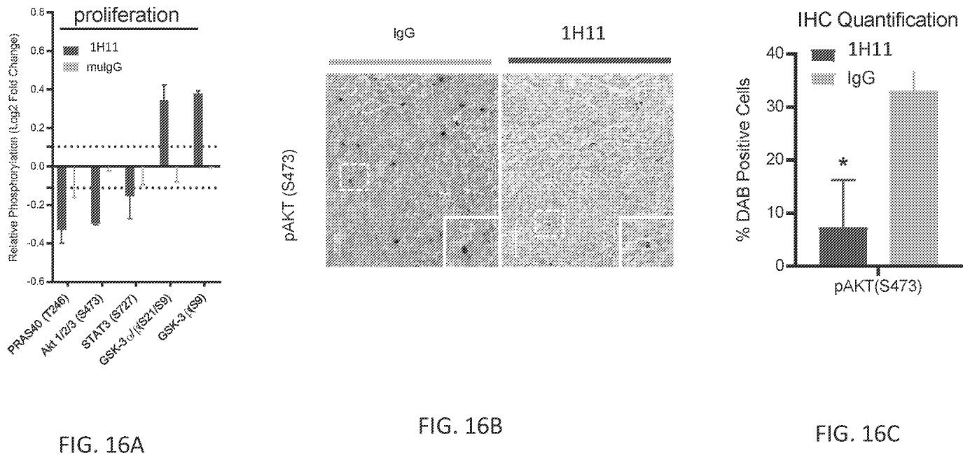

To further investigate the mechanism by which anti-CSP-1 antibodies decrease proliferation, OVCAR8 cells were treated for 10 mins with 125 nM of either control IgG antibody or murine anti-CSP-1 antibody, and the phosphorylation of signaling proteins involved proliferation, such as proteins of the Akt/PRAS40 pathway, was assessed. As shown in FIG. 16A, the phosphorylation levels of Akt/PRAS40 pathway proteins was altered (increased or decreased) upon treatment with murine anti-CSP-1 antibody, as compared to control IgG antibody. Moreover, immunohistochemistry analysis showed that the level of phosphorylated Akt signaling protein in MiaPACA2 tumor tissue isolated from mice treated with murine anti-CSP-1 antibody is decreased, as compared to treatment with IgG control antibody (FIGS. 16B and 16C). These results show that treatment with anti-CSP-1 antibodies results in decreased cell proliferation via Akt/PRAS40 pathway in vitro and in vivo.

In sum, these results demonstrate that treatment with anti-CSP-1 antibodies (such as, Ab51) results in the arrest of proliferation of different cancer types in vitro and in vivo, further underlining the potential for using humanized anti-CSP-1 antibodies as cancer therapeutics.

### Example 7: Treatment with Humanized Anti-CSP-1 Antibody Decreases Migration of Cholangiocarcinoma Cells

To further investigate the anti-tumor action of humanized anti-CSP-1 antibodies (such as, Ab51), a scratch or wound assay was performed using the cholangiocarcinoma cell line HuCCT1.

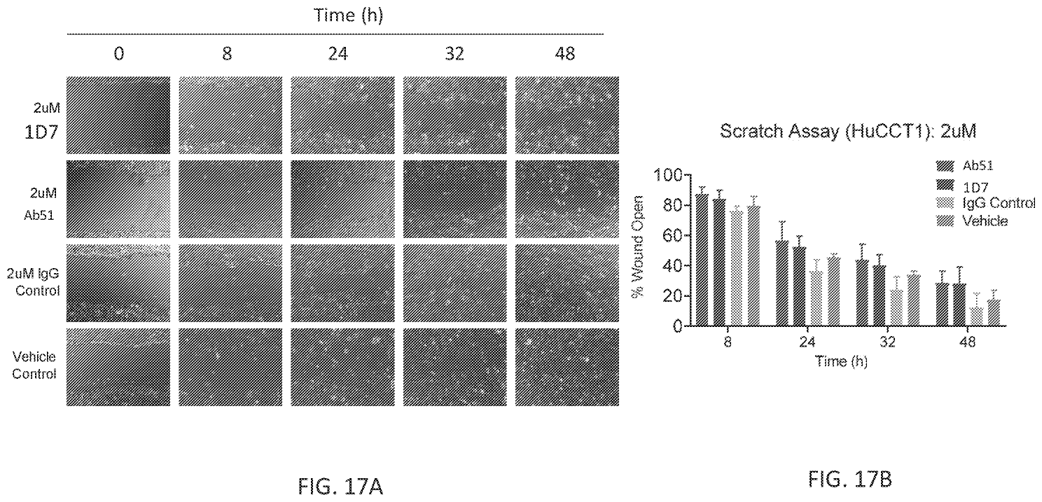

HuCCT1 cells were grown in a monolayer to confluence, and a scratch or wound was made to create a cell-free zone at timepoint 0. The migration of cells into the scratched region was monitored with time, which reflects the migration capability of the cells. FIG. 17A shows images of the scratched region in the cell culture that was untreated or treated with Ab51, a murine anti-CSP1 antibody, or an IgG control antibody over time. The images show that by 48 hours, the wounds in untreated or control antibody-treated cells were completed closed. In sharp contrast, cancer cells treated with murine anti-CSP-1 antibody or Ab51 remained open even after 48 hours. As shown in FIG. 17B, this effect is quantifiable, and the wound was more open when the cells were treated with Ab51, as compared to the control antibody.

These results indicate that treatment of cancer cells with humanized anti-CSP-1 antibodies, such as, Ab51, decreases the migration capability, and therefore, the metastatic capability of the cancer cells.

### Example 8: Treatment with Humanized Anti-CSP-1 Antibody Decreases Migration of Ovarian Cancer Cells

To investigate whether the effect of humanized anti-CSP-1 antibodies on cancer cell migration extends to other cancer types, a similar scratch or wound assay as described above was performed using the ovarian cancer cell line SKOV3.

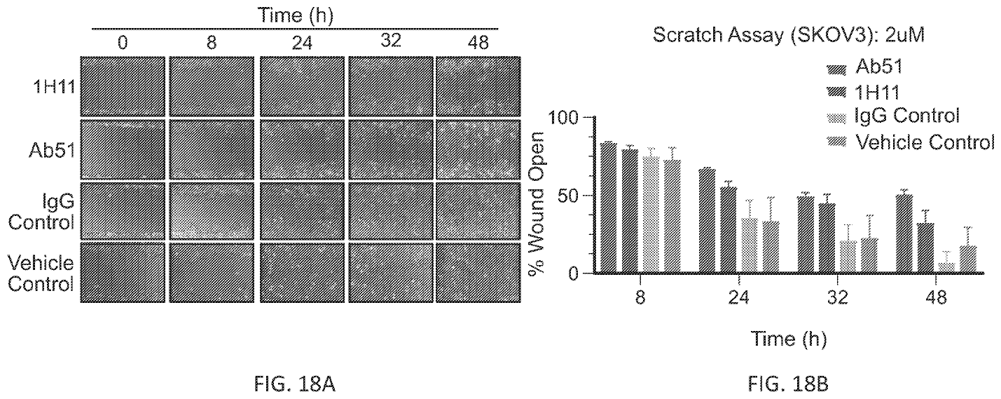

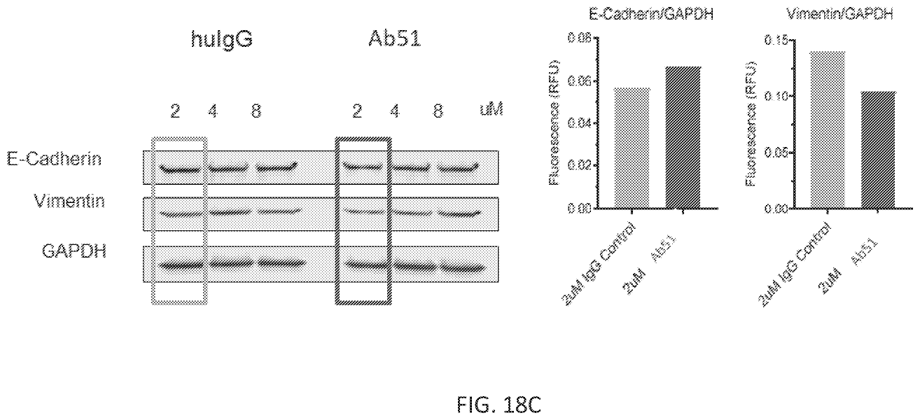

FIG. 18A shows images of the scratched region in cell culture that was untreated or treated with Ab51, a murine anti-CSP1 antibody, or an IgG control antibody over time. While by 48 hours, the wounds in untreated or control antibody-treated cell cultures were completed closed, wounds in cell cultures treated with murine anti-CSP-1 antibody or Ab51 remained open even after 48 hours. The quantitation of the open wound shown in FIG. 18B further highlights the difference in wound closing between Ab51-treated cells and untreated or control antibody-treated cells.

To further investigate the mechanism by which humanized anti-CSP-1 antibodies decrease cancer cell migration, the expression of migration-associated proteins, such as, E-cadherin and vimentin in ovarian cancer cells was evaluated by Western Blot after treatment with Ab51. As shown in FIG. 18C, levels of E-cadherin and vimentin were reduced in cells treated with Ab51, as compared to control antibody-treated cells, indicating that anti-CSP-1 antibodies, such as Ab51, decrease cancer cell migration by changing the cancer cell proteome.

In sum, these results demonstrate that treatment of cancer cells with humanized anti-CSP-1 antibodies, such as, Ab51 decreases the migration capability, and therefore stunts the metastatic potential, of different types of cancer cells.

### Example 9: Treatment with Anti-CSP-1 Antibody Induces Tumor Cell Apoptosis

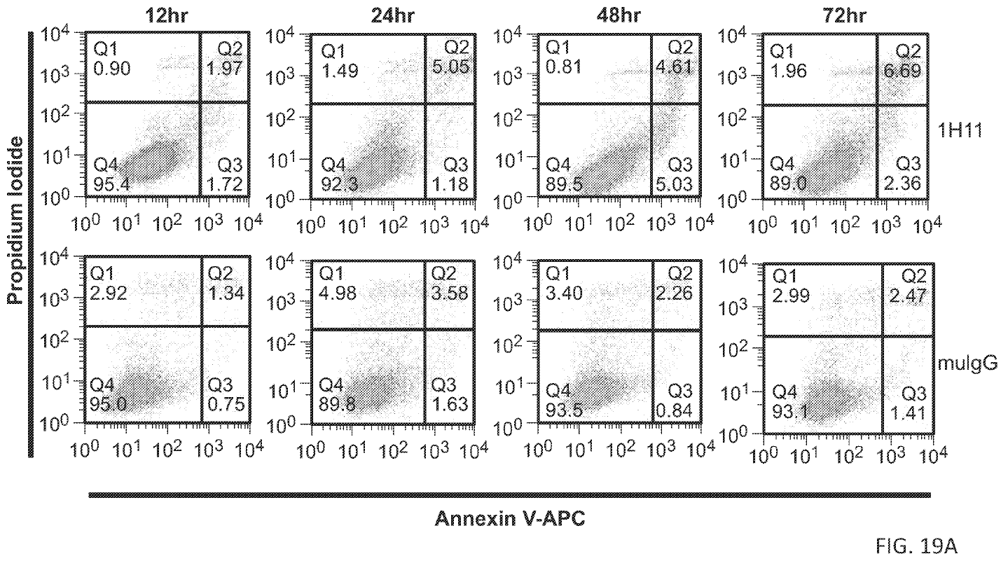

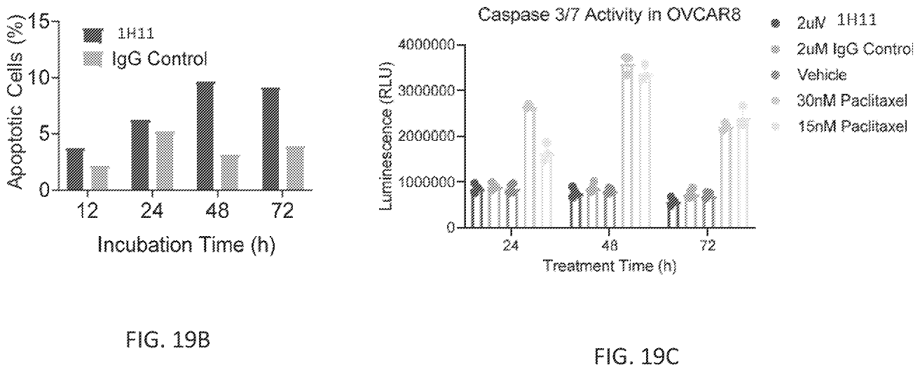

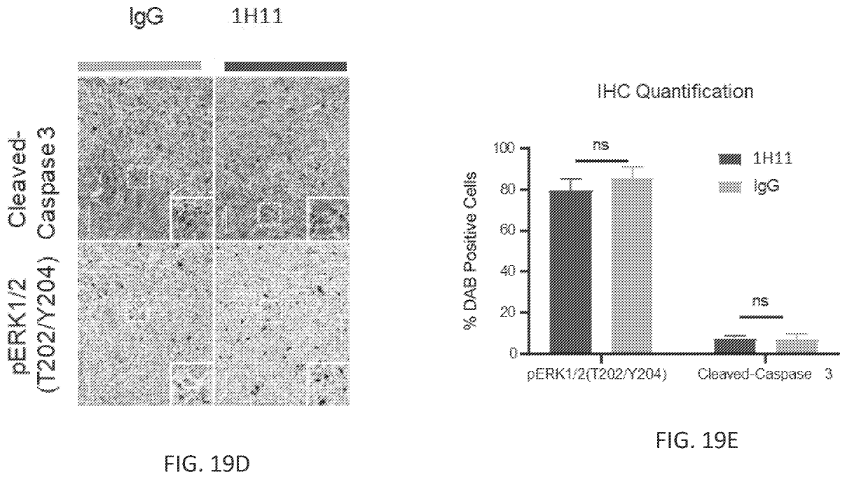

To evaluate the effect on anti-CSP-1 antibodies on cell survival, ovarian cancer cells OVCAR8 were treated with murine anti-CSP-1 antibody, and the number of apoptotic cells was evaluated over time using flow cytometry using propidium iodide (PI) and annexin V as markers, as shown in FIG. 19A and as quantitated in FIG. 19B. However, the activity of caspase 3 and caspase 7, which promote apoptosis was not as high in anti-CSP-1 antibody-treated cells, as compared to the positive control (paclitaxel-treated cells) in vitro (FIG. 19C) or in vivo (FIGS. 19D and 19E). These results demonstrate that about 10% of tumor cells that are treated with murine anti-CSP-1 antibody undergo apoptosis.

### Example 10: Humanized Anti-CSP-1 Antibody Elicits Minimal Complement-Dependent Cytotoxicity (CDC) Activity

Complement-dependent cytotoxicity (CDC) was assessed by culturing OVCAR8 cells. Serial dilutions of Ab51, recombinant Cetuximab (a control antibody not known to act via CDC), or IgG1 negative isotype control antibody were added in the presence of human complement and the viability of the cells was analyzed by Propidium Iodide (P.I.) staining. Recombinant Rituxan (anti-CD20 antibody) treatment of Raji target cells was used as a technical positive control.

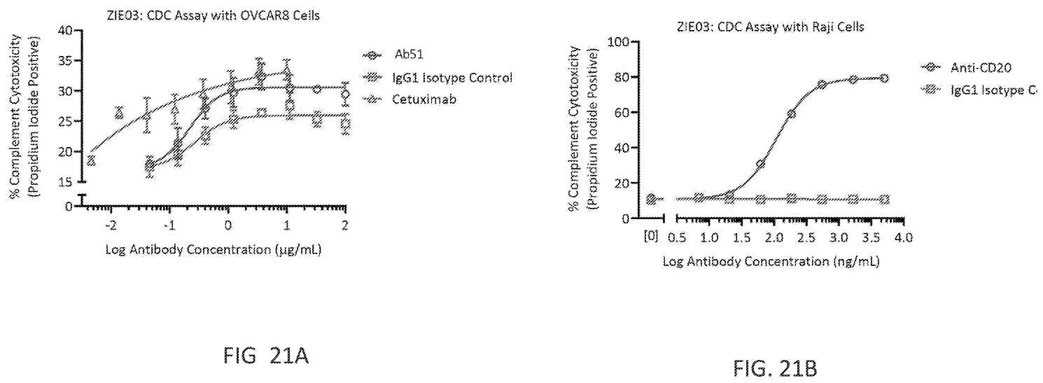

As shown in FIG. 21B, CDC bioactivity was observed on Raji target cells with Rituxan treatment. As shown in FIG. 21A and listed in Table 3, treatment with Ab51 or recombinant Cetuximab showed only minimally higher CDC activity, as compared to IgG1 isotype control antibody. These results show that treatment with humanized anti-CSP-1 antibody results in minimal CDC activity, and thereby limits off-target toxicity.

TABLE 3

| Test Article | EC₅₀ (μg/mL) | Max P.I. % |
|---|---|---|
| huZB131 | 0.22 | 32.28 |
| Cetuximab | ~1.11 × 10−12 | 33.47 |
| Human IgG1 Isotype | 0.27 | 27.55 |
| Control | | |

### Example 11: Humanized Anti-CSP-1 Antibody does not Elicit Antibody-Dependent Cellular Cytotoxicity (ADCC)

The cytotoxicity dose-response was evaluated by culturing ViaFluor 405-labeled OVCAR8 target cells treated with a range of concentrations of Ab51, recombinant Cetuximab, or IgG1 negative isotype control antibody in the presence of human PBMCs from three donors and analyzing the viability of target cells by flow cytometry after labeling with the viability dye, propidium iodide (P.I.). Recombinant Rituxan (anti-CD20 antibody) treatment of Raji target cells was used as a technical positive control.

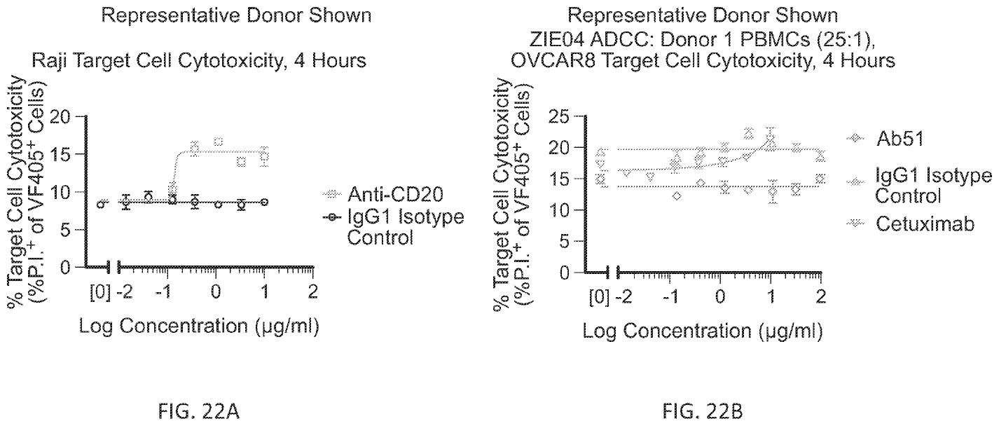

As shown in FIG. 22A, ADCC bioactivity was observed in Raji target cells with Rituxan treatment in the presence of PBMCs from all three donors. In contrast, ADCC bioactivity was not observed upon treatment of OVCAR8 cells with Ab51 or IgG control antibody (FIG. 22B). Treatment with recombinant Cetuximab increased the percentage of P.I. staining in the OVCAR8 target at the highest tested concentration with PBMCs from all donors, suggesting that higher concentrations of Cetuximab are required to induce ADCC with OVCAR8 target cells. Overall, these results show that treatment of cancer cells with anti-CSP-1 antibodies does not elicit Antibody-Dependent Cellular Cytotoxicity (ADCC).

### Example 12. Clinical Study

A Phase ½ dose escalation study of an anti-CSP-1 antibody is conducted in patients with solid tumors that are likely to express CSP. The study is comprised of a dose escalation stage, which will enroll patients with solid tumors who have failed all available therapies or are not eligible for standard of care; and an expansion stage which will consist of up to 3 cohorts: Cohort A, advanced or metastatic pancreatic cancer (pancreatic ductal; adenocarcinoma) who have failed or are not eligible for standard of care; Cohort B, advanced or metastatic ovarian cancer of the serous type (ovarian serous adenocarcinoma; ovarian serous cystadenocarcinoma) who have failed or are not eligible for standard of care; and Cohort C, advanced or metastatic biliary cancer (intrahepatic, extrahepatic, gallbladder) who have failed or are not eligible for standard of care.

The primary objectives of the dose escalation stage are to characterize the safety and tolerability of the antibody when administered via intravenous (IV) infusion in patients with advanced solid tumors for whom no standard treatment is further available; and to identify the recommended Phase 2 dose (RP2D) of the antibody when administered via IV infusion. The primary objectives of the expansion stage are to further characterize the safety and tolerability of the antibody when administered via IV infusion at the RP2D; to evaluate preliminary efficacy of the antibody when administered via IV infusion at the RP2D in multiple predefined cohorts using the following, in accordance with Response Evaluation Criteria in Solid Tumors (RECIST; Eisenhauer, et al. Eur J Cancer 2009, 45(2):228-247):
Overall response rate (ORR),
Duration of response (DoR), and
Progression-free survival (PFS).

Additional efficacy endpoints include clinical benefit rate, defined as the complete response (CR)/partial response (PR) and stable disease (SD) greater than 4 months, and reduction of tumor markers, where appropriate.

Secondary objectives for both stages of the study include: to characterize the pharmacokinetics (PK), pharmacodynamics (PD), and immune-relatedity of the antibody when administered via IV infusion; to explore the relationships between the PK, PD, adverse event (AE) profile, and clinical activity of the antibody when administered via IV infusion; and to determine the immunogenicity (anti-drug antibodies) of the antibody. Pharmacokinetic endpoints include pharmacokinetic profile determination, including maximum plasma concentration (Cmax), time to Cmax (Tmax), area under the plasma concentration-time curve (AUC) at various timepoints, apparent total body clearance of the drug from plasma (CL), and apparent volume of distribution (Vss and Vz). Pharmacodynamic endpoints include immune subset analysis (CD4, CD8), CSP IHC and P53 mutation/deletion, and others.

The exploratory objectives for both stages of this study include: to evaluate predictive biomarkers of response or resistance to the antibody (cytokines IL-6, IL-1β; immune subset analysis (CD4, CD8); proliferation markers (Ki67, P27, P21; cancer-specific plectin immunohistochemistry assay; target engagement assay; and others); and to evaluate preliminary efficacy of the antibody when administered via IV infusion at the RP2D in multiple predefined cohorts using ORR and DoR in accordance with immune-related Response Evaluation Criteria in Solid Tumors (iRECIST, Seymour et al, Lancet Oncology 2017, 18(3):143-152).

Approximately 12 to 24 patients will be enrolled in the Dose Escalation Stage; the total number of patients will depend on the dose level at which the RP2D is defined. Patients who meet the eligibility criteria during Screening will enter the treatment period. The test antibody will be given via IV infusion (rate not less than 60 minute ([+15 min]) every week. Patients will be treated until disease progression or unacceptable toxicities occur.

Once the RP2D has been established, the Dose Expansion stage will begin to enroll subjects by cohort. For each expansion cohort, subjects will be enrolled in two stages. The first stage includes an evaluation of 12 patients. If zero of the 12 patients respond, then accrual in that cohort will end. If 1 or more of 12 patients have a CR or PR, then accrual will continue until a total of 37 patients with measurable disease have been enrolled. Additional patients may be added to ensure 37 subjects with measurable disease are available for evaluation.

At the Dose Escalation Stage, a standard 3+3 dose escalation scheme will be employed to determine the maximum tolerated dose (MTD) or the maximum administered dose (MAD), and to establish the RP2D of the antibody. During the Dose Escalation Stage, patients will be treated with the test antibody at increasing dose levels, beginning with a starting dose level (DL0) of 0.3 mg/kg once weekly, up to a maximum dose level (DL4) of 15 mg/kg once weekly. Assessment for escalation will occur at the end of each cycle, based upon the available safety and PK data of the current dose.

| Dose Level | Dose (mg/kg) | Dosing Regimen (Frequency) |
|---|---|---|
| 0* | 0.3 | Once weekly |
| 1 | 1 | Once weekly |
| 2 | 3 | Once weekly |
| 3 | 9 | Once weekly |
| 4 | 15 | Once weekly |

*The starting dose level will be 0.3 mg/kg for 3 weeks, after which a safety assessment will be conducted prior to escalating to the next dose level.

Each cycle will be 3 weeks. Based upon the nonclinical safety profile, all proposed doses are below the recommended safe starting dose per FDA guidance, i.e., below one-sixth of the highest non-severely toxic dose. The MTD is defined as the highest dose level of antibody at which no more than 1 of 6 patients experiences a dose limiting toxicity (DLT). If the MTD is not reached, the MAD will be used as the highest dose.

At the Expansion Stage, it is expected up to 111 patients will receive the antibody 1 at the RP2D established in the Dose Escalation Stage of the study in at least 3 pre-defined cohorts. If efficacy is observed in the first 3 expansion Cohorts, additional Cohorts can be opened. For each expansion cohort, subjects will be enrolled in two stages. The first stage includes an evaluation of 12 patients. If zero of the 12 patients respond, then accrual in that cohort will end. If 1 or more of 12 patients have a CR or PR, then accrual will continue until a total of 37 patients with measurable disease have been enrolled. Additional patients may be added to ensure 37 subjects with measurable disease are available for evaluation.

The test antibody, Ab51, will be provided as a sterile solution, suitable for injection. It will be provided in single-use vials containing 2.5 mL of 50 mg/mL antibody formulated in 20 mM Histidine-HCl pH 5.5, 8% (w/v) sucrose, 10 mM Methionine, 0.02% (w/v) polysorbate 80.

At the Dose Escalation Stage, the antibody will be administered via IV infusion (rate not less than 60 minutes [+15 min]) with a starting dose of 0.3 mg/kg weekly, up to a maximum dose level of 15 mg/kg weekly. At the Expansion Stage, the antibody will be administered via IV infusion (rate not less than 60 minutes [+15 min]) at the RP2D determined in the Dose Escalation Stage.

### Example 12: Treatment with an Antibody-Drug Conjugate (ADO Comprising a Humanized Anti-CSP1 Antibody and a Chemotherapeutic Drug Results in Cancer Cell Death

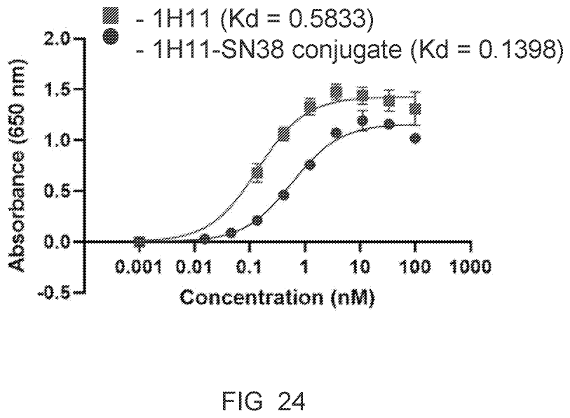

The binding characteristics of an antibody-drug conjugate comprising the murine anti-CSP-1 antibody, 1H11 and a chemotherapeutic drug, SN38 was compared to that of 1H11 alone. FIG. 24 shows the results from an ELISA assay indicating that conjugating 1H11 with SN38 does not significantly alter its binding capability to the target region of CSP-1 (Sec8). Because the 1H11-SN38 conjugate exhibits strong binding to its target CSP-1 region, the conjugate may be used to specifically target tumor cells expressing CSP-1.

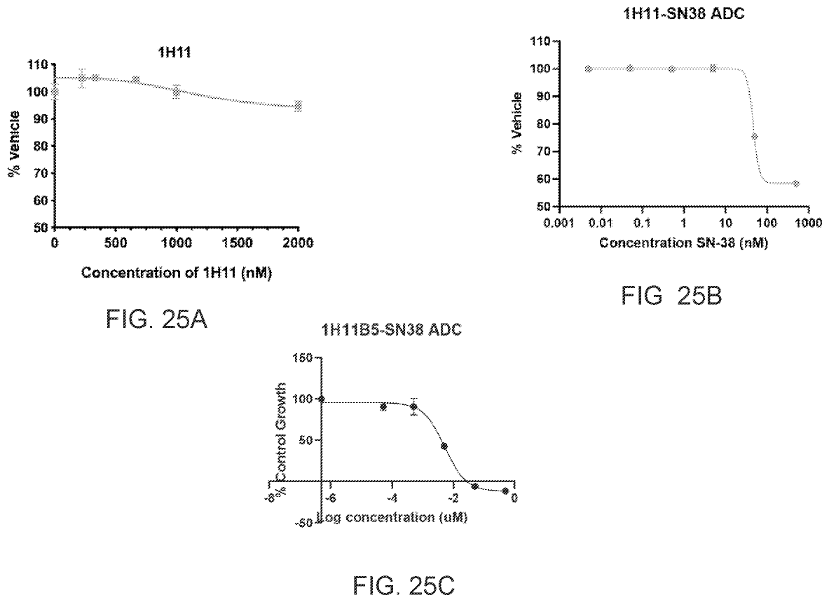

To test whether the 1H11-SN38 conjugate is capable of targeting and inducing cell death in cancer cells in vitro, OVCAR8 ovarian cancer cells or MIA PaCa-2 pancreatic cancer cells were treated with varying concentrations of 1H11 alone or the conjugate comprising 1H11 and SN38, and percentage of live cancer cells was evaluated using the CellTiter-Glo® proliferation assay. As shown in FIGS. 25A-25C, treatment of OVCAR8 (FIG. 25B) or MIA PaCa-2 cells (FIG. 25C) with the 1H11-SN38 conjugate resulted in a sharp decrease in the percentage of live cancer cells, indicating that the antibody-drug conjugate of 1H11 and SN38 is able to induce the death of the cancer cells.

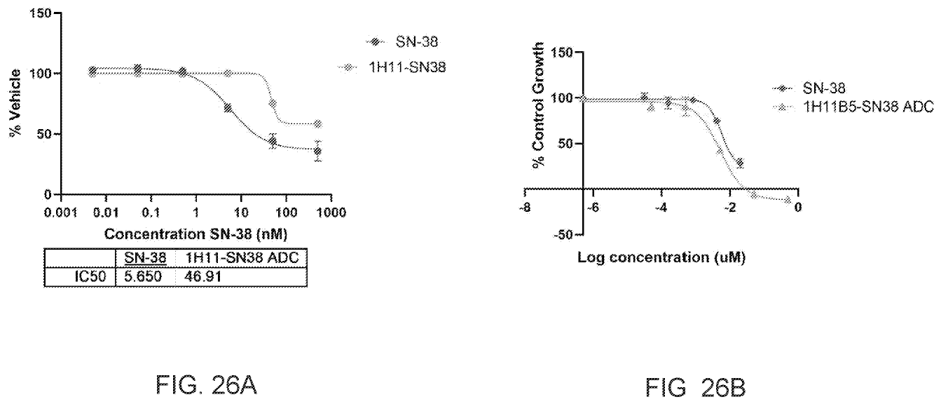

In contrast, treatment of OVCAR8 cells with 1H11 alone did not result in a similar reduction in the percentage of live cancer cells (FIG. 25A), indicating that the cytotoxic effect seen in FIGS. 25B and 25C was dependent on exposure to SN38 (conjugated with 1H11). Additionally, treatment of OVCAR8 or MIA PaCa-2 cells with the 1H11-SN38 conjugate resulted in a similar decrease in the percentage of live cancer cells, as compared to SN38 alone (FIGS. 26A and 26B). These data demonstrate that the conjugation of 1H11 to SN38 does not hinder the cytotoxic effects of SN38 on cancer cells. Rather, without wishing to be bound by theory, the cytotoxicity of the 1H11-SN38 conjugate is comparable to free SN38 likely because the 1H11-SN38 conjugate targets the cancer cells more efficiently through binding to CSP-1 on the surface of OVCAR8 or MIA PaCa-2 cells.

### Example 13: Humanized Anti-CSP1 Antibody Localizes within Tumor Tissue

The therapeutic effects of an antibody (such as the humanized anti-CSP1 antibodies disclosed herein) are promoted not only by their localization to the outside of a tumor, but also by their distribution within the tumor tissue.

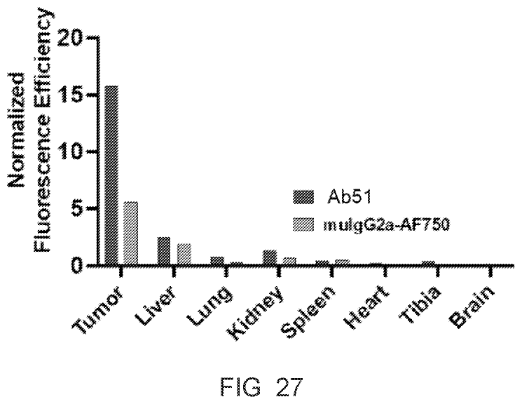

To test whether the humanized anti-CSP1 antibody, Ab51 is capable of localizing to CSP-1 containing tumor cells, a syngeneic mouse model of ductal adenocarcinoma of the pancreas (PDAC) was injected with (i) a Alexa Fluor 750 (AF750) dye labelled reverse chimeric antibody (comprising the complementarity determining regions (CDRs) of Ab51 on a murine IgG2a constant region), or (ii) a AF750 dye-labeled control murine IgG2a antibody. As shown in FIG. 27, the chimeric antibody comprising the CDRs of Ab51 was seen to locate to tumor cells more effectively than the control antibody. Moreover, Ab51 selectively localizes to the tumor cells, showing minimal, non-specific localization to other cell types, such as lung, kidney and spleen.

To test whether the humanized anti-CSP1 antibody, Ab51 is capable of localizing within tumor tissue, nude mice xenografted with extrahepatic cholangiocarcinoma (ECC) WITT cells were intravenously administered 5 mg/kg Ab51 or a control IgG1 antibody. Tumor tissue was collected 3 days after the last dose, and analyzed by immunohistochemistry (IHC) in the following manner.

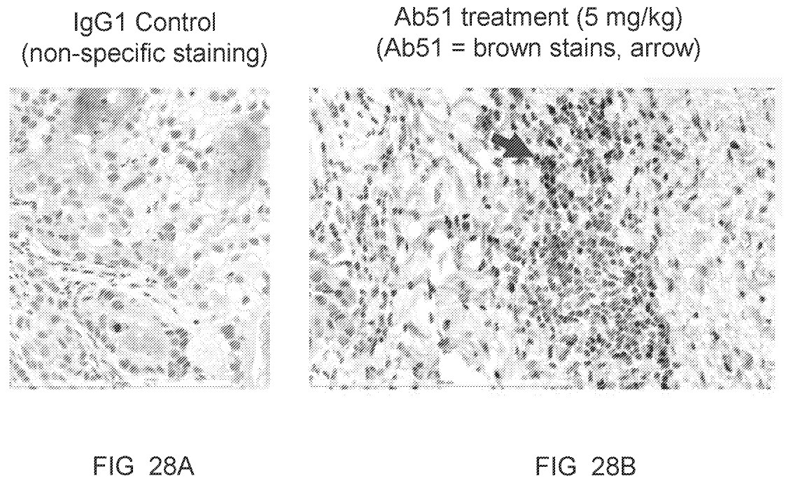

The tumors were excised, fixed and embedded in paraffin. Tumor sections were sliced and stained with an anti-huIgG1 antibody (which binds to the constant domain of Ab51 or to the control IgG1 that is present in the tumor section) and hematoxylin to stain nuclei, followed by chromogenic detection (visible on a light microscope). The anti-huIgG1 antibody is conjugated to HRP (horse radish peroxidase), which oxidizes a reagent added to the staining called DAB (3,3′-Diaminobenzidine). The formation of an insoluble brown-colored precipitate indicates the presence of HRP, and thereby the presence of the anti-human IgG1 antibody bound to the Ab51 in the tumor tissue. Therefore, the presence of brown staining (indicated by arrow in FIG. 28B) indicates the presence of Ab51 positive cells. As shown in FIG. 28B, Ab51 staining is detected as being present within the tumor tissue, while staining for the control IgG1 antibody is not detected (FIG. 28A).

These data demonstrate that the anti-CSP1 antibodies disclosed herein, such as Ab51, are capable of not just locating to the surface of a tumor, but also penetrating the tumor tissue and localizing to tumor cells located within the tumor. This ability of anti-CSP1 antibodies is extremely valuable in the context of treatment of solid tumors with the anti-CSP1 antibodies disclosed herein.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed is:

1. An antibody or antigen-binding fragment thereof that binds cancer specific plectin-1 (CSP-1), comprising a heavy chain variable region, and a light chain variable region,
wherein the heavy chain variable region comprises a heavy chain complementary determining region 1 (HCDR1), a HCDR2, and a HCDR3 and
wherein the light chain variable region comprises a light chain complementary determining region 1 (LCDR1), a LCDR2, and a LCDR3,
wherein the HCDR1 comprises an amino acid sequence according to SEQ ID NO: 104 (GFTFSRYG), the HCDR2 comprises an amino acid sequence according to SEQ ID NO: 115 (ISIGGTYT), the HCDR3 comprises an amino acid sequence according to SEQ ID NO: 134 (ARRGYGX₅YSYYGX₁₁DY, wherein X₅ is N, S, or Q; and X₁₁ is M, L, I, or V);
the LCDR1 comprises an amino acid sequence according to SEQ ID NO: 135 (KSLLHSX₁₀GITY, wherein X₁₀ is N, V, D, Q, S or E); the LCDR2 comprises an amino acid sequence according to SEQ ID NO: 136 (QX₂S, wherein X₂ is M, L, V, or I); and the LCDR3 comprises an amino acid sequence according to SEQ ID NO: 133 (AQNLELPLT).

2. The antibody or antigen binding fragment thereof of claim 1, wherein the HCDR3 comprises an amino acid sequence of any one of SEQ ID NOs: 125-131, the LCDR1 comprises an amino acid sequence according to any one of SEQ ID NOs: 107-112, and the LCDR2 comprises an amino acid sequence according to any one of SEQ ID NOs: 120-123.

3. The antibody or antigen binding fragment thereof of claim 1 or 2, wherein the HCDR3 comprises an amino acid sequence according to SEQ ID NO: 131, the LCDR1 comprises an amino acid sequence according to SEQ ID NO: 111, and the LCDR2 comprises an amino acid sequence according to SEQ ID NO: 120.

4. The antibody or antigen binding fragment thereof of claim 1 or 2, wherein the antibody or antigen binding fragment comprises a heavy chain variable region having at least about 90% identity to any one of SEQ ID Nos: 16-39, 43-46, and 49-51.

5. The antibody or antigen binding fragment thereof of claim 1 or 2, wherein the antibody or antigen binding fragment comprises a light chain variable region having at least about 90% identity to any one of SEQ ID Nos: 67-90, 94-97, and 100-102.

6. The antibody or antigen binding fragment thereof of any one of claims 1-3, wherein the antibody or antigen binding fragment comprises a heavy chain variable region having at least about 90% identity to SEQ ID NO: 51.

7. The antibody or antigen binding fragment thereof of any one of claims 1-6, wherein the antibody or fragment comprises a light chain variable region having at least about 90% identity to SEQ ID NO: 102.

8. The antibody or antigen binding fragment thereof of any one of claims 1-7, comprising a heavy chain amino acid sequence according to SEQ ID NO: 186 and a light chain amino acid sequence according to SEQ ID NO: 187.

9. An antibody or antigen-binding fragment thereof that binds cancer specific plectin-1 (CSP-1), comprising a heavy chain variable region, and a light chain variable region,
wherein the heavy chain variable region comprises a heavy chain complementary determining region 1 (HCDR1), a HCDR2, and a HCDR3 and
wherein the light chain variable region comprises a light chain complementary determining region 1 (LCDR1), a LCDR2, and a LCDR3,
wherein the HCDR1 comprises an amino acid sequence according to SEQ ID NO: 103 (GYTFTDYS); the HCDR2 comprises an amino acid sequence according to SEQ ID NO: 139 (IX₁₂TETGEP, wherein X₁₂ is N or Q); the HCDR3 comprises an amino acid sequence according to SEQ ID NO: 124 (APGGFAY); wherein the LCDR1 comprises an amino acid sequence according to SEQ ID NO: 140 (KSLLHSX₁₃GNTY, wherein X₁₃ is N or S); wherein the LCDR2 comprises an amino acid sequence according to SEQ ID NO: 141 (RX₁₄S, wherein X₁₄ is M, V, L, or I); and wherein LCDR3 comprises an amino acid sequence according to SEQ ID NO: 132 (MQHLEYPLT).

10. The antibody or antigen-binding fragment thereof of claim 9, wherein the HCDR2 comprises the amino acid sequence of SEQ ID NO: 113 or 114; the LCDR1 comprises the amino acid sequence of SEQ ID NO: 105 or 106; and the LCDR2 comprises the amino acid sequence of any one of SEQ ID NOs: 116-119.

11. The antibody or antigen binding fragment thereof of claim 9 or 10, wherein the antibody or antigen binding fragment comprises a heavy chain variable region having at least about 90% identity to any one of SEQ ID Nos: 1-16, 40-42, 47 and 48.

12. The antibody or antigen binding fragment thereof of any one of claims 9-11, wherein the antibody or antigen binding fragment comprises a light chain variable region having at least about 90% identity to any one of SEQ ID Nos: 52-66, 91-93, 98 and 99.

13. An antibody or antigen-binding fragment thereof that binds cancer specific plectin-1 (CSP-1), comprising a heavy chain variable region, and a light chain variable region,
wherein the heavy chain variable region comprises a heavy chain complementary determining region 1 (HCDR1), a HCDR2, and a HCDR3 and
wherein the light chain variable region comprises a light chain complementary determining region 1 (LCDR1), a LCDR2, and a LCDR3, wherein
the HCDR1 comprises the amino acid sequence of SEQ ID NO: 103 or 104, the HCDR2 comprises the amino acid sequence of SEQ ID NO: 113, 114, or 115, the HCDR3 comprises an amino acid sequence selected from the group consisting of SEQ ID Nos: 124-131, the LCDR1 comprises an amino acid sequence selected from the group consisting of SEQ ID Nos: 105-112, the LCDR2 comprises an amino acid sequence selected from the group consisting of SEQ ID Nos 116-123, and the LCDR3 comprises the amino acid sequence of SEQ ID NO: 132 or 133.

14. The antibody or antigen binding fragment thereof of claim 13, wherein
1) the HCDR1, HCDR2, and HCDR3 comprise the amino acid sequence of SEQ ID NOs: 103, 113, and 124, respectively, and LCDR1, LCDR2, and LCDR3 comprise the amino acid sequence of SEQ ID NOs: 105, 116, and 132, respectively;
2) the HCDR1, HCDR2, and HCDR3 comprise the amino acid sequence of SEQ ID NOs: 103, 114, and 124, respectively, and LCDR1, LCDR2, and LCDR3 comprise the amino acid sequence of SEQ ID NOs: 105, 116, and 132, respectively;
3) the HCDR1, HCDR2, and HCDR3 comprise the amino acid sequence of SEQ ID NOs: 103, 113, and 124, respectively, and LCDR1, LCDR2, and LCDR3 comprise the amino acid sequence of SEQ ID NOs: 106, 116, and 132, respectively;
4) the HCDR1, HCDR2, and HCDR3 comprise the amino acid sequence of SEQ ID NOs: 103, 113, and 124, respectively, and LCDR1, LCDR2, and LCDR3 comprise the amino acid sequence of SEQ ID NOs: 105, 117, and 132, respectively;
5) the HCDR1, HCDR2, and HCDR3 comprise the amino acid sequence of SEQ ID NOs: 103, 113, and 124, respectively, and LCDR1, LCDR2, and LCDR3 comprise the amino acid sequence of SEQ ID NOs: 105, 118, and 132, respectively;
6) the HCDR1, HCDR2, and HCDR3 comprise the amino acid sequence of SEQ ID NOs: 103, 113, and 124, respectively, and LCDR1, LCDR2, and LCDR3 comprise the amino acid sequence of SEQ ID NOs: 105, 119, and 132, respectively;
7) the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 107, 120, and 133, respectively;
8) the HCDR1, HCDR2, and HCDR3 comprise the amino acid sequence of SEQ ID NOs: 104, 115, and 126, respectively, and LCDR1, LCDR2, and LCDR3 comprise the amino acid sequence of SEQ ID NOs: 107, 120, and 133, respectively;
9) the HCDR1, HCDR2, and HCDR3 comprise the amino acid sequence of SEQ ID NOs: 104, 115, and 127, respectively, and LCDR1, LCDR2, and LCDR3 comprise the amino acid sequence of SEQ ID NOs: 107, 120, and 133, respectively;
10) the HCDR1, HCDR2, and HCDR3 comprise the amino acid sequence of SEQ ID NOs: 104, 115, and 128, respectively, and LCDR1, LCDR2, and LCDR3 comprise the amino acid sequence of SEQ ID NOs: 107, 120, and 133, respectively;
11) the HCDR1, HCDR2, and HCDR3 comprise the amino acid sequence of SEQ ID NOs: 104, 115, and 129, respectively, and LCDR1, LCDR2, and LCDR3 comprise the amino acid sequence of SEQ ID NOs: 107, 120, and 133, respectively;
12) the HCDR1, HCDR2, and HCDR3 comprise the amino acid sequence of SEQ ID NOs: 104, 115, and 130, respectively, and LCDR1, LCDR2, and LCDR3 comprise the amino acid sequence of SEQ ID NOs: 107, 120, and 133, respectively;
13) the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 108, 120, and 133, respectively;
14) the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 109, 120, and 133, respectively;
15) the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 110, 120, and 133, respectively;
16) the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 111, 120, and 133, respectively;
17) the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 112, 120, and 133, respectively;
18) the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 107, 121, and 133, respectively;
19) the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 107, 122, and 133, respectively;
20) the HCDR1, HCDR2, and HCDR3 comprise the amino acid sequence of SEQ ID NOs: 104, 115, and 125, respectively, and LCDR1, LCDR2, and LCDR3 comprise the amino acid sequence of SEQ ID NOs: 107, 123, and 133, respectively;
21) the HCDR1, HCDR2, and HCDR3 comprise the amino acid sequence of SEQ ID NOs: 104, 115, and 131, respectively, and LCDR1, LCDR2, and LCDR3 comprise the amino acid sequence of SEQ ID NOs: 107, 120, and 133, respectively; OR
22) the HCDR1, HCDR2, and HCDR3 comprise the amino acid sequence of SEQ ID NOs: 104, 115, and 131, respectively, and LCDR1, LCDR2, and LCDR3 comprise the amino acid sequence of SEQ ID NOs: 111, 120, and 133, respectively.

15. The antibody or antigen binding fragment thereof of claim 13 or 14, wherein the antibody or antigen binding fragment thereof comprises:
1) a heavy chain variable region having at least about 90% identity to SEQ ID NO: 1 and a light chain variable region having at least about 90% identity to SEQ ID NO: 52;
2) a heavy chain variable region having at least about 90% identity to SEQ ID NO: 2 and a light chain variable region having at least about 90% identity to SEQ ID NO: 53;
3) a heavy chain variable region having at least about 90% identity to SEQ ID NO: 3 and a light chain variable region having at least about 90% identity to SEQ ID NO: 54;
4) a heavy chain variable region having at least about 90% identity to SEQ ID NO: 4 and a light chain variable region having at least about 90% identity to SEQ ID NO: 55;
5) a heavy chain variable region having at least about 90% identity to SEQ ID NO: 5 and a light chain variable region having at least about 90% identity to SEQ ID NO: 56;
6) a heavy chain variable region having at least about 90% identity to SEQ ID NO: 6 and a light chain variable region having at least about 90% identity to SEQ ID NO: 57;
7) a heavy chain variable region having at least about 90% identity to SEQ ID NO: 7 and a light chain variable region having at least about 90% identity to SEQ ID NO: 58;
8) a heavy chain variable region having at least about 90% identity to SEQ ID NO: 8 and a light chain variable region having at least about 90% identity to SEQ ID NO: 59;
9) a heavy chain variable region having at least about 90% identity to SEQ ID NO: 9 and a light chain variable region having at least about 90% identity to SEQ ID NO: 60;
10) a heavy chain variable region having at least about 90% identity to SEQ ID NO: 10 and a light chain variable region having at least about 90% identity to SEQ ID NO: 61;
11) a heavy chain variable region having at least about 90% identity to SEQ ID NO: 11 and a light chain variable region having at least about 90% identity to SEQ ID NO: 62;
12) a heavy chain variable region having at least about 90% identity to SEQ ID NO: 12 and a light chain variable region having at least about 90% identity to SEQ ID NO: 63;
13) a heavy chain variable region having at least about 90% identity to SEQ ID NO: 13 and a light chain variable region having at least about 90% identity to SEQ ID NO: 64;
14) a heavy chain variable region having at least about 90% identity to SEQ ID NO: 14 and a light chain variable region having at least about 90% identity to SEQ ID NO: 65;
15) a heavy chain variable region having at least about 90% identity to SEQ ID NO: 15 and a light chain variable region having at least about 90% identity to SEQ ID NO: 66;
16) a heavy chain variable region having at least about 90% identity to SEQ ID NO: 16 and a light chain variable region having at least about 90% identity to SEQ ID NO: 67;
17) a heavy chain variable region having at least about 90% identity to SEQ ID NO: 17 and a light chain variable region having at least about 90% identity to SEQ ID NO: 68;
18) a heavy chain variable region having at least about 90% identity to SEQ ID NO: 18 and a light chain variable region having at least about 90% identity to SEQ ID NO: 69;
19) a heavy chain variable region having at least about 90% identity to SEQ ID NO: 19 and a light chain variable region having at least about 90% identity to SEQ ID NO: 70;
20) a heavy chain variable region having at least about 90% identity to SEQ ID NO: 20 and a light chain variable region having at least about 90% identity to SEQ ID NO: 71;
21) a heavy chain variable region having at least about 90% identity to SEQ ID NO: 21 and a light chain variable region having at least about 90% identity to SEQ ID NO: 72;
22) a heavy chain variable region having at least about 90% identity to SEQ ID NO: 22 and a light chain variable region having at least about 90% identity to SEQ ID NO: 73;
23) a heavy chain variable region having at least about 90% identity to SEQ ID NO: 23 and a light chain variable region having at least about 90% identity to SEQ ID NO: 74;
24) a heavy chain variable region having at least about 90% identity to SEQ ID NO: 24 and a light chain variable region having at least about 90% identity to SEQ ID NO: 75;
25) a heavy chain variable region having at least about 90% identity to SEQ ID NO: 25 and a light chain variable region having at least about 90% identity to SEQ ID NO: 76;
26) a heavy chain variable region having at least about 90% identity to SEQ ID NO: 26 and a light chain variable region having at least about 90% identity to SEQ ID NO: 77;
27) a heavy chain variable region having at least about 90% identity to SEQ ID NO: 27 and a light chain variable region having at least about 90% identity to SEQ ID NO: 78;
28) a heavy chain variable region having at least about 90% identity to SEQ ID NO: 28 and a light chain variable region having at least about 90% identity to SEQ ID NO: 79;
29) a heavy chain variable region having at least about 90% identity to SEQ ID NO: 29 and a light chain variable region having at least about 90% identity to SEQ ID NO: 80;
30) a heavy chain variable region having at least about 90% identity to SEQ ID NO: 30 and a light chain variable region having at least about 90% identity to SEQ ID NO: 81;
31) a heavy chain variable region having at least about 90% identity to SEQ ID NO: 31 and a light chain variable region having at least about 90% identity to SEQ ID NO: 82;
32) a heavy chain variable region having at least about 90% identity to SEQ ID NO: 32 and a light chain variable region having at least about 90% identity to SEQ ID NO: 83;
33) a heavy chain variable region having at least about 90% identity to SEQ ID NO: 33 and a light chain variable region having at least about 90% identity to SEQ ID NO: 84;
34) a heavy chain variable region having at least about 90% identity to SEQ ID NO: 34 and a light chain variable region having at least about 90% identity to SEQ ID NO: 85;
35) a heavy chain variable region having at least about 90% identity to SEQ ID NO: 35 and a light chain variable region having at least about 90% identity to SEQ ID NO: 86;
36) a heavy chain variable region having at least about 90% identity to SEQ ID NO: 36 and a light chain variable region having at least about 90% identity to SEQ ID NO: 87;
37) a heavy chain variable region having at least about 90% identity to SEQ ID NO: 37 and a light chain variable region having at least about 90% identity to SEQ ID NO: 88;
38) a heavy chain variable region having at least about 90% identity to SEQ ID NO: 38 and a light chain variable region having at least about 90% identity to SEQ ID NO: 89;
39) a heavy chain variable region having at least about 90% identity to SEQ ID NO: 39 and a light chain variable region having at least about 90% identity to SEQ ID NO: 90;
40) a heavy chain variable region having at least about 90% identity to SEQ ID NO: 40 and a light chain variable region having at least about 90% identity to SEQ ID NO: 91;
41) a heavy chain variable region having at least about 90% identity to SEQ ID NO: 41 and a light chain variable region having at least about 90% identity to SEQ ID NO: 92;
42) a heavy chain variable region having at least about 90% identity to SEQ ID NO: 42 and a light chain variable region having at least about 90% identity to SEQ ID NO: 93;
43) a heavy chain variable region having at least about 90% identity to SEQ ID NO: 43 and a light chain variable region having at least about 90% identity to SEQ ID NO: 94;
44) a heavy chain variable region having at least about 90% identity to SEQ ID NO: 44 and a light chain variable region having at least about 90% identity to SEQ ID NO: 95;
45) a heavy chain variable region having at least about 90% identity to SEQ ID NO: 45 and a light chain variable region having at least about 90% identity to SEQ ID NO: 96;
46) a heavy chain variable region having at least about 90% identity to SEQ ID NO: 46 and a light chain variable region having at least about 90% identity to SEQ ID NO: 97;
47) a heavy chain variable region having at least about 90% identity to SEQ ID NO: 47 and a light chain variable region having at least about 90% identity to SEQ ID NO: 98;
48) a heavy chain variable region having at least about 90% identity to SEQ ID NO: 48 and a light chain variable region having at least about 90% identity to SEQ ID NO: 99;
49) a heavy chain variable region having at least about 90% identity to SEQ ID NO: 49 and a light chain variable region having at least about 90% identity to SEQ ID NO: 100;
50) a heavy chain variable region having at least about 90% identity to SEQ ID NO: 50 and a light chain variable region having at least about 90% identity to SEQ ID NO: 101; OR 51) a heavy chain variable region having at least about 90% identity to SEQ ID NO: 51 and a light chain variable region having at least about 90% identity to SEQ ID NO: 102.

16. The antibody or antigen binding fragment thereof of any one of claims 1-15, wherein the antibody or fragment thereof is a monoclonal antibody, a Fab, F(ab′)2, Fab′, scFv, or a single domain antibody (sdAb).

17. The antibody or antigen binding fragment thereof of any one of claims 1-16, wherein the antibody comprises a human IgG1 or IgG4 domain.

18. The antibody or antigen binding fragment thereof of claim 17, comprising an IgG1 domain having a constant heavy domain according to SEQ ID NO: 188.

19. The antibody or antigen binding fragment thereof of claim 17, comprising an IgG4 domain having a constant heavy domain according to SEQ ID NO: 190.

20. The antibody or antigen binding fragment thereof of claim 16, wherein the antibody or fragment thereof is a monoclonal antibody.

21. The antibody or antigen binding fragment thereof of claim 16, wherein the antibody or fragment thereof is a single domain antibody (sdAb).

22. The antibody or antigen binding fragment thereof of any one of claims 1-21, wherein the antibody or fragment thereof has a binding affinity for CSP-1 of about 50 nM or less.

23. The antibody or antigen binding fragment thereof of any one of claims 1-22, wherein the antibody causes internalization of CSP-1 upon binding.

24. The antibody or antigen binding fragment thereof of any one of claims 1-23, wherein the binding of the antibody to CSP-1 induces immune-related cell death.

25. An epitope on the surface of a cancer cell consisting of at least one amino acid residue of SEQ ID NO: 185.

26. An antibody or antigen-binding fragment thereof that specifically binds to the epitope of claim 25.

27. A protein complex comprising the antibody or antigen-binding fragment thereof of any one of claims 1-24 and 26, bound to CSP-1 on the surface of a cancer cell.

28. A composition, comprising the antibody or antigen-binding fragment of any one of claims 1-24 and 26.

29. A composition, comprising the antibody or antigen-binding fragment of any one of claims 1-25 and 26, and a therapeutic agent.

30. The composition of claim 29, wherein the antibody or antigen-binding fragment is conjugated to the therapeutic agent.

31. The composition of claim 29 or 30, wherein the therapeutic agent is a drug.

32. The composition of claim 31, wherein the drug is a chemotherapeutic drug.

33. The composition of claim 31, wherein the drug is SN38, fludarabine, ibrutinib, fostamatinib, lenalidomide, thalidomide, rituximab, cyclophosphamide, doxorubicin, vincristine, prednisone, rituximab, cyclophosphamide, doxorubicin hydroxydaunomycin, vincristine, oncovin, prednisone, bendamustine, gemcitabine, oxaliplatin, cyclophosphamide, vincristine, vinblastine, anthracycline, daunorubicin, daunomycin, doxorubicin, actinomycin dactinomycin, bleomycin, clofarabine, nelarabine, cladribine, asparaginase, methotrexate, or pralatrexate.

34. A method of forming a protein complex of claim 27, the method comprising administering the antibody or antigen-binding fragment thereof to a subject, wherein the subject has a cancer, and wherein the cancer is associated with cell surface expression of CSP-1.

35. A method of forming a protein complex of claim 27, the method comprising contacting the CSP-1 with the antibody or antigen-binding fragment thereof in vivo in a subject with a cancer, wherein the cancer is associated with cell surface expression of CSP-1.

36. An isolated nucleic acid molecule encoding the antibody or antigen-binding fragment thereof of any one of claims 1-24 and 26.

37. An expression vector comprising a nucleic acid segment encoding the antibody or antigen-binding fragment thereof of any one of claims 1-24 and 26.

38. A recombinant host cell comprising the expression vector of claim 37.

39. A method for treating a cancer comprising administering to a subject in need thereof the antibody or antigen binding fragment thereof of any one of claims 1-24 and 26, the composition of any one of claims 28-33, the nucleic acid molecule of claim 36, or the expression vector of claim 37.

40. A method for inducing immune-related cell death of cancer cells in a subject having cancer, the method comprising administering to the subject an antibody or fragment thereof of any one of claims 1-24 and 26, the composition of any one of claims 28-33, the nucleic acid molecule of claim 36, or the expression vector of claim 37.

41. A method for inhibiting proliferation and/or migration of cancer cells in a subject having cancer, the method comprising administering to the subject an antibody or fragment thereof of any one of claims 1-24 and 26, the composition of any one of claims 28-33, the nucleic acid molecule of claim 36, or the expression vector of claim 37.

42. The method of any one of claims 34, 35, and 39-41, wherein the cancer is selected from the group consisting of pancreatic cancer, ovarian cancer, gastric cancer, colorectal carcinoma, cholangiocarcinoma, Head and neck squamous cell carcinoma (HNSCC), triple negative breast cancer (TNBR), prostate cancer, and non-small cell lung cancer (NSCLC).

43. The method of claim 40, wherein the immune-related cell death induced by the administration of the antibody or antigen binding fragment thereof increases anti-cancer T cell responses in the subject.

44. The method of any one of claims 34, 35, and 39-41, wherein the administration of the antibody or antigen binding fragment thereof elicits at least a 2-fold increase in effector memory T cells in the subject.

45. The method of any one of claims 34, 35, and 39-41, wherein the administration of the antibody or antigen binding fragment thereof increases macrophage infiltration of tumors in the subject.


SEQUENCE LISTING (395 sequences)

SEQ ID NO: 1 - protein, 114 aa, synthetic construct
  REGION 1..114: note = VH sequence
QIQLVQSGPELKKPGETVKISCKASGYTFTDYSMHWVKQAPGKGLKWMGWINTETGEPTYADDFKGRFAFSLETSASTAYLQINNLKNEDTATYFCAPGGFAYWGQGTLVTVSA

SEQ ID NO: 2 - protein, 114 aa, synthetic construct
  REGION 1..114: note = VH sequence
QIQLVQSGPELKKPGETVKISCKASGYTFTDYSMHWVKQAPGKGLKWMGWIQTETGEPTYADDFKGRFAFSLETSASTAYLQINNLKNEDTATYFCAPGGFAYWGQGTLVTVSA

SEQ ID NO: 3 - protein, 114 aa, synthetic construct; identical to SEQ ID NO: 1
  REGION 1..114: note = VH sequence

SEQ ID NO: 4 - protein, 114 aa, synthetic construct; identical to SEQ ID NO: 1
  REGION 1..114: note = VH sequence

SEQ ID NO: 5 - protein, 114 aa, synthetic construct; identical to SEQ ID NO: 1
  REGION 1..114: note = VH sequence

SEQ ID NO: 6 - protein, 114 aa, synthetic construct; identical to SEQ ID NO: 1
  REGION 1..114: note = VH sequence

SEQ ID NO: 7 - protein, 114 aa, synthetic construct
  REGION 1..114: note = VH sequence
QIQLVQSGPEVKKPGASVKISCKASGYTFTDYSMHWVKQAPGQGLKWMGWINTETGEPTYADDFKGRFTFTLDTSTSTAYLEISSLRSEDTATYFCAPGGFAYWGQGTLVTVSS

SEQ ID NO: 8 - protein, 114 aa, synthetic construct
  REGION 1..114: note = VH sequence
QIQLVQSGPEVKKPGASVKISCKASGYTFTDYSMHWVRQAPGQGLEWMGWINTETGEPTYAQKFQGRFTFTLDTSTSTAYLEISSLRSEDTAVYFCAPGGFAYWGQGTLVTVSS

SEQ ID NO: 9 - protein, 114 aa, synthetic construct; identical to SEQ ID NO: 1
  REGION 1..114: note = VH sequence

SEQ ID NO: 10 - protein, 114 aa, synthetic construct; identical to SEQ ID NO: 1
  REGION 1..114: note = VH sequence

SEQ ID NO: 11 - protein, 114 aa, synthetic construct; identical to SEQ ID NO: 7
  REGION 1..114: note = VH sequence

SEQ ID NO: 12 - protein, 114 aa, synthetic construct; identical to SEQ ID NO: 7
  REGION 1..114: note = VH sequence

SEQ ID NO: 13 - protein, 114 aa, synthetic construct; identical to SEQ ID NO: 8
  REGION 1..114: note = VH sequence

SEQ ID NO: 14 - protein, 114 aa, synthetic construct
  REGION 1..114: note = VH sequence
QIQLVQSGPGLKQPGGSVRISCAASGYTFTDYSMHWVKQAPGKGLKWMGWINTETGEPTYADDFKGRFTFSLDTSKSTAYLQINSLRAEDTATYFCAPGGFAYWGQGTLVTVSS

SEQ ID NO: 15 - protein, 114 aa, synthetic construct; identical to SEQ ID NO: 14
  REGION 1..114: note = VH sequence

SEQ ID NO: 16 - protein, 122 aa, synthetic construct
  REGION 1..122: note = VH sequence
EVQLVESGGDLVKPGGSLKLSCAASGFTFSRYGMSWVRQTPDKRLEWVATISIGGTYTYYPDSMKGRFTISRDNAKNTLYLQMSSLKSEDTAMYYCARRGYGNYSYYGMDYWGQGTSVTVSS

SEQ ID NO: 17 - protein, 122 aa, synthetic construct
  REGION 1..122: note = VH sequence
EVQLVESGGDLVKPGGSLKLSCAASGFTFSRYGMSWVRQTPDKRLEWVATISIGGTYTYYPDSMKGRFTISRDNAKNTLYLQMSSLKSEDTAMYYCARRGYGSYSYYGMDYWGQGTSVTVSS

SEQ ID NO: 18 - protein, 122 aa, synthetic construct
  REGION 1..122: note = VH sequence
EVQLVESGGDLVKPGGSLKLSCAASGFTFSRYGMSWVRQTPDKRLEWVATISIGGTYTYYPDSMKGRFTISRDNAKNTLYLQMSSLKSEDTAMYYCARRGYGQYSYYGMDYWGQGTSVTVSS

SEQ ID NO: 19 - protein, 122 aa, synthetic construct
  REGION 1..122: note = VH sequence
EVQLVESGGDLVKPGGSLKLSCAASGFTFSRYGMSWVRQTPDKRLEWVATISIGGTYTYYPDSMKGRFTISRDNAKNTLYLQMSSLKSEDTAMYYCARRGYGNYSYYGLDYWGQGTSVTVSS

SEQ ID NO: 20 - protein, 122 aa, synthetic construct
  REGION 1..122: note = VH sequence
EVQLVESGGDLVKPGGSLKLSCAASGFTFSRYGMSWVRQTPDKRLEWVATISIGGTYTYYPDSMKGRFTISRDNAKNTLYLQMSSLKSEDTAMYYCARRGYGNYSYYGIDYWGQGTSVTVSS

SEQ ID NO: 21 - protein, 122 aa, synthetic construct
  REGION 1..122: note = VH sequence
EVQLVESGGDLVKPGGSLKLSCAASGFTFSRYGMSWVRQTPDKRLEWVATISIGGTYTYYPDSMKGRFTISRDNAKNTLYLQMSSLKSEDTAMYYCARRGYGNYSYYGVDYWGQGTSVTVSS

SEQ ID NO: 22 - protein, 122 aa, synthetic construct; identical to SEQ ID NO: 16
  REGION 1..122: note = VH sequence

SEQ ID NO: 23 - protein, 122 aa, synthetic construct; identical to SEQ ID NO: 16
  REGION 1..122: note = VH sequence

SEQ ID NO: 24 - protein, 122 aa, synthetic construct; identical to SEQ ID NO: 16
  REGION 1..122: note = VH sequence

SEQ ID NO: 25 - protein, 122 aa, synthetic construct; identical to SEQ ID NO: 16
  REGION 1..122: note = VH sequence

SEQ ID NO: 26 - protein, 122 aa, synthetic construct; identical to SEQ ID NO: 16
  REGION 1..122: note = VH sequence

SEQ ID NO: 27 - protein, 122 aa, synthetic construct; identical to SEQ ID NO: 16
  REGION 1..122: note = VH sequence

SEQ ID NO: 28 - protein, 122 aa, synthetic construct; identical to SEQ ID NO: 16
  REGION 1..122: note = VH sequence

SEQ ID NO: 29 - protein, 122 aa, synthetic construct; identical to SEQ ID NO: 16
  REGION 1..122: note = VH sequence

SEQ ID NO: 30 - protein, 122 aa, synthetic construct
  REGION 1..122: note = VH sequence
EVQLVESGGEVVKPGASLKLSCKASGFTFSRYGMSWVRQAPGQRLEWVATISIGGTYTYYPDSMKGRFTITRDNSTNTLYLEMSSLRSEDTAMYYCARRGYGNYSYYGMDYWGQGTLVTVSS

SEQ ID NO: 31 - protein, 122 aa, synthetic construct
  REGION 1..122: note = VH sequence
EVQLVESGGGLVQPGGSLRLSCAASGFTFSRYGMSWVRQAPGKRLEWVATISIGGTYTYYPDSMKGRFTISRDNSKNTLYLQMNSLRAEDTAMYYCARRGYGNYSYYGMDYWGQGTLVTVSS

SEQ ID NO: 32 - protein, 122 aa, synthetic construct
  REGION 1..122: note = VH sequence
EVQLVESGGGLVQPGGSLRLSCAASGFTFSRYGMSWVRQAPGKGLEWVATISIGGTYTYYPDSMKGRFTISRDNSKNTLYLQMNSLRAEDTAVYYCARRGYGNYSYYGMDYWGQGTLVTVSS

SEQ ID NO: 33 - protein, 122 aa, synthetic construct; identical to SEQ ID NO: 16
  REGION 1..122: note = VH sequence

SEQ ID NO: 34 - protein, 122 aa, synthetic construct; identical to SEQ ID NO: 16
  REGION 1..122: note = VH sequence

SEQ ID NO: 35 - protein, 122 aa, synthetic construct; identical to SEQ ID NO: 30
  REGION 1..122: note = VH sequence

SEQ ID NO: 36 - protein, 122 aa, synthetic construct; identical to SEQ ID NO: 31
  REGION 1..122: note = VH sequence

SEQ ID NO: 37 - protein, 122 aa, synthetic construct; identical to SEQ ID NO: 31
  REGION 1..122: note = VH sequence

SEQ ID NO: 38 - protein, 122 aa, synthetic construct; identical to SEQ ID NO: 32
  REGION 1..122: note = VH sequence

SEQ ID NO: 39 - protein, 122 aa, synthetic construct; identical to SEQ ID NO: 32
  REGION 1..122: note = VH sequence

SEQ ID NO: 40 - protein, 114 aa, synthetic construct
  REGION 1..114: note = VH sequence
QVQLVQSGSELKKPGASVKVSCKASGYTFTDYSMNWVRQAPGQGLEWMGWINTETGEPTYAQGFTGRFVFSLDTSVSTAYLQISSLKAEDTAVYYCAPGGFAYWGQGTLVTVSS

SEQ ID NO: 41 - protein, 114 aa, synthetic construct
  REGION 1..114: note = VH sequence
QIQLVQSGSELKKPGASVKVSCKASGYTFTDYSMHWVRQAPGQGLEWMGWINTETGEPTYAQGFTGRFVFSLDTSVSTAYLQISSLKAEDTAVYFCAPGGFAYWGQGTLVTVSS

SEQ ID NO: 42 - protein, 114 aa, synthetic construct; identical to SEQ ID NO: 41
  REGION 1..114: note = VH sequence

SEQ ID NO: 43 - protein, 122 aa, synthetic construct
  REGION 1..122: note = VH sequence
EVQLVESGGGLVKPGGSLRLSCAASGFTFSRYGMNWVRQAPGKGLEWVSSISIGGTYTYYADSVKGRFTISRDNAKNSLYLQMNSLRAEDTAVYYCARRGYGNYSYYGMDYWGQGTLVTVSS

SEQ ID NO: 44 - protein, 122 aa, synthetic construct; identical to SEQ ID NO: 43
  REGION 1..122: note = VH sequence

SEQ ID NO: 45 - protein, 122 aa, synthetic construct; identical to SEQ ID NO: 43
  REGION 1..122: note = VH sequence

SEQ ID NO: 46 - protein, 122 aa, synthetic construct
  REGION 1..122: note = VH sequence
EVQLVESGGGLVKPGGSLRLSCAASGFTFSRYGMSWVRQAPGKGLEWVSSISIGGTYTYYADSVKGRFTISRDNAKNSLYLQMNSLRAEDTAVYYCARRGYGNYSYYGMDYWGQGTLVTVSS

SEQ ID NO: 47 - protein, 114 aa, synthetic construct; identical to SEQ ID NO: 41
  REGION 1..114: note = VH sequence

SEQ ID NO: 48 - protein, 114 aa, synthetic construct; identical to SEQ ID NO: 41
  REGION 1..114: note = VH sequence

SEQ ID NO: 49 - protein, 122 aa, synthetic construct
  REGION 1..122: note = VH sequence
EVQLVESGGGLVQPGGSLRLSCAASGFTFSRYGMSWVRQAPGKRLEWVATISIGGTYTYYPDSMKGRFTISRDNSKNTLYLQMNSLRAEDTAMYYCARRGYGQYSYYGMDYWGQGTLVTVSS

SEQ ID NO: 50 - protein, 122 aa, synthetic construct
  REGION 1..122: note = VH sequence
EVQLVESGGGLVQPGGSLRLSCAASGFTFSRYGMSWVRQAPGKRLEWVATISIGGTYTYYPDSMKGRFTISRDNSKNTLYLQMNSLRAEDTAMYYCARRGYGQYSYYGLDYWGQGTLVTVSS

SEQ ID NO: 51 - protein, 122 aa, synthetic construct; identical to SEQ ID NO: 50
  REGION 1..122: note = VH sequence

SEQ ID NO: 52 - protein, 112 aa, synthetic construct
  REGION 1..112: note = VL sequence
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPLTFGAGTKLELK

SEQ ID NO: 53 - protein, 112 aa, synthetic construct; identical to SEQ ID NO: 52
  REGION 1..112: note = VL sequence

SEQ ID NO: 54 - protein, 112 aa, synthetic construct
  REGION 1..112: note = VL sequence
DIVMTQAAPSVPVTPGESVSISCRSSKSLLHSSGNTYLYWFLQRPGQSPQLLIYRMSNLASGVPDRFSGSGSGTAFTLRISRVEAEDVGVYYCMQHLEYPLTFGAGTKLELK

SEQ ID NO: 55 - protein, 112 aa, synthetic construct
  REGION 1..112: note = VL sequence
DIVMTQAAPSVPVTPGESVSISCRSSKSLLHSNGNTYLYWFLQRPGQSPQLLIYRVSNLASGVPDRFSGSGSGTAFTLRISRVEAEDVGVYYCMQHLEYPLTFGAGTKLELK

SEQ ID NO: 56 - protein, 112 aa, synthetic construct
  REGION 1..112: note = VL sequence
DIVMTQAAPSVPVTPGESVSISCRSSKSLLHSNGNTYLYWFLQRPGQSPQLLIYRLSNLASGVPDRFSGSGSGTAFTLRISRVEAEDVGVYYCMQHLEYPLTFGAGTKLELK

SEQ ID NO: 57 - protein, 112 aa, synthetic construct
  REGION 1..112: note = VL sequence
DIVMTQAAPSVPVTPGESVSISCRSSKSLLHSNGNTYLYWFLQRPGQSPQLLIYRISNLASGVPDRFSGSGSGTAFTLRISRVEAEDVGVYYCMQHLEYPLTFGAGTKLELK

SEQ ID NO: 58 - protein, 112 aa, synthetic construct; identical to SEQ ID NO: 52
  REGION 1..112: note = VL sequence

SEQ ID NO: 59 - protein, 112 aa, synthetic construct; identical to SEQ ID NO: 52
  REGION 1..112: note = VL sequence

SEQ ID NO: 60 - protein, 112 aa, synthetic construct
  REGION 1..112: note = VL sequence
DIVMTQSAPSVPVTPGQSVSISCRSSKSLLHSNGNTYLYWFLQKPGQSPQLLIYRMSNRASGVPDRFSGSGSGTDFTLKISRVEAEDVGVYYCMQHLEYPLTFGQGTKLEIK

SEQ ID NO: 61 - protein, 112 aa, synthetic construct
  REGION 1..112: note = VL sequence
DIVMTQSAPSLPVTPGQSVSISCRSSKSLLHSNGNTYLYWFLQKPGQSPQLLIYRMSNRASGVPDRFSGSGSGTDFTLKISRVEAEDVGVYYCMQHLEYPLTFGQGTKLEIK

SEQ ID NO: 62 - protein, 112 aa, synthetic construct; identical to SEQ ID NO: 60
  REGION 1..112: note = VL sequence

SEQ ID NO: 63 - protein, 112 aa, synthetic construct; identical to SEQ ID NO: 61
  REGION 1..112: note = VL sequence

SEQ ID NO: 64 - protein, 112 aa, synthetic construct; identical to SEQ ID NO: 60
  REGION 1..112: note = VL sequence

SEQ ID NO: 65 - protein, 112 aa, synthetic construct; identical to SEQ ID NO: 60
  REGION 1..112: note = VL sequence

SEQ ID NO: 66 - protein, 112 aa, synthetic construct; identical to SEQ ID NO: 61
  REGION 1..112: note = VL sequence

SEQ ID NO: 67 - protein, 112 aa, synthetic construct
  REGION 1..112: note = VL sequence
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPLTFGAGTKLELK

SEQ ID NO: 68 - protein, 112 aa, synthetic construct; identical to SEQ ID NO: 67
  REGION 1..112: note = VL sequence

SEQ ID NO: 69 - protein, 112 aa, synthetic construct; identical to SEQ ID NO: 67
  REGION 1..112: note = VL sequence

SEQ ID NO: 70 - protein, 112 aa, synthetic construct; identical to SEQ ID NO: 67
  REGION 1..112: note = VL sequence

SEQ ID NO: 71 - protein, 112 aa, synthetic construct; identical to SEQ ID NO: 67
  REGION 1..112: note = VL sequence

SEQ ID NO: 72 - protein, 112 aa, synthetic construct; identical to SEQ ID NO: 67
  REGION 1..112: note = VL sequence

SEQ ID NO: 73 - protein, 112 aa, synthetic construct
  REGION 1..112: note = VL sequence
DIVMTQAAFSNPVTLGTSASISCRSSKSLLHSSGITYLYWYLQKPGQSPQLLIYQMSNLASGVPDRFSSSGSGTDFTLRISRVEAEDVGVYYCAQNLELPLTFGAGTKLELK

SEQ ID NO: 74 - protein, 112 aa, synthetic construct
  REGION 1..112: note = VL sequence
DIVMTQAAFSNPVTLGTSASISCRSSKSLLHSVGITYLYWYLQKPGQSPQLLIYQMSNLASGVPDRFSSSGSGTDFTLRISRVEAEDVGVYYCAQNLELPLTFGAGTKLELK

SEQ ID NO: 75 - protein, 112 aa, synthetic construct
  REGION 1..112: note = VL sequence
DIVMTQAAFSNPVTLGTSASISCRSSKSLLHSDGITYLYWYLQKPGQSPQLLIYQMSNLASGVPDRFSSSGSGTDFTLRISRVEAEDVGVYYCAQNLELPLTFGAGTKLELK

SEQ ID NO: 76 - protein, 112 aa, synthetic construct
  REGION 1..112: note = VL sequence
DIVMTQAAFSNPVTLGTSASISCRSSKSLLHSQGITYLYWYLQKPGQSPQLLIYQMSNLASGVPDRFSSSGSGTDFTLRISRVEAEDVGVYYCAQNLELPLTFGAGTKLELK

SEQ ID NO: 77 - protein, 112 aa, synthetic construct
  REGION 1..112: note = VL sequence
DIVMTQAAFSNPVTLGTSASISCRSSKSLLHSEGITYLYWYLQKPGQSPQLLIYQMSNLASGVPDRFSSSGSGTDFTLRISRVEAEDVGVYYCAQNLELPLTFGAGTKLELK

SEQ ID NO: 78 - protein, 112 aa, synthetic construct
  REGION 1..112: note = VL sequence
DIVMTQAAFSNPVTLGTSASISCRSSKSLLHSNGITYLYWYLQKPGQSPQLLIYQVSNLASGVPDRFSSSGSGTDFTLRISRVEAEDVGVYYCAQNLELPLTFGAGTKLELK

SEQ ID NO: 79 - protein, 112 aa, synthetic construct
  REGION 1..112: note = VL sequence
DIVMTQAAFSNPVTLGTSASISCRSSKSLLHSNGITYLYWYLQKPGQSPQLLIYQLSNLASGVPDRFSSSGSGTDFTLRISRVEAEDVGVYYCAQNLELPLTFGAGTKLELK

SEQ ID NO: 80 - protein, 112 aa, synthetic construct
  REGION 1..112: note = VL sequence
DIVMTQAAFSNPVTLGTSASISCRSSKSLLHSNGITYLYWYLQKPGQSPQLLIYQISNLASGVPDRFSSSGSGTDFTLRISRVEAEDVGVYYCAQNLELPLTFGAGTKLELK

SEQ ID NO: 81 - protein, 112 aa, synthetic construct; identical to SEQ ID NO: 67
  REGION 1..112: note = VL sequence

SEQ ID NO: 82 - protein, 112 aa, synthetic construct; identical to SEQ ID NO: 67
  REGION 1..112: note = VL sequence

SEQ ID NO: 83 - protein, 112 aa, synthetic construct; identical to SEQ ID NO: 67
  REGION 1..112: note = VL sequence

SEQ ID NO: 84 - protein, 112 aa, synthetic construct
  REGION 1..112: note = VL sequence
DIVMTQSALSNPVTLGQSASISCRSSKSLLHSNGITYLYWYLQKPGQSPQLLIYQMSNRASGVPDRFSSSGSGTDFTLKISRVEAEDVGVYYCAQNLELPLTFGQGTKLEIK

SEQ ID NO: 85 - protein, 112 aa, synthetic construct
  REGION 1..112: note = VL sequence
DIVMTQSALSLPVTLGQSASISCRSSKSLLHSNGITYLYWYLQKPGQSPQLLIYQMSNRASGVPDRFSSSGSGTDFTLKISRVEAEDVGVYYCAQNLELPLTFGQGTKLEIK

SEQ ID NO: 86 - protein, 112 aa, synthetic construct; identical to SEQ ID NO: 84
  REGION 1..112: note = VL sequence

SEQ ID NO: 87 - protein, 112 aa, synthetic construct; identical to SEQ ID NO: 84
  REGION 1..112: note = VL sequence

SEQ ID NO: 88 - protein, 112 aa, synthetic construct; identical to SEQ ID NO: 85
  REGION 1..112: note = VL sequence

SEQ ID NO: 89 - protein, 112 aa, synthetic construct; identical to SEQ ID NO: 84
  REGION 1..112: note = VL sequence

SEQ ID NO: 90 - protein, 112 aa, synthetic construct; identical to SEQ ID NO: 85
  REGION 1..112: note = VL sequence

SEQ ID NO: 91 - protein, 112 aa, synthetic construct
  REGION 1..112: note = VL sequence
DIVMTQSPLSLPVTPGEPASISCRSSKSLLHSNGNTYLYWFLQKPGQSPQLLIYRMSNRASGVPDRFSGSGSGTDFTLKISRVEAEDVGVYYCMQHLEYPLTFGQGTKLEIK

SEQ ID NO: 92 - protein, 112 aa, synthetic construct; identical to SEQ ID NO: 91
  REGION 1..112: note = VL sequence

SEQ ID NO: 93 - protein, 112 aa, synthetic construct
  REGION 1..112: note = VL sequence
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPLTFGAGTKLELK

SEQ ID NO: 94 - protein, 112 aa, synthetic construct
  REGION 1..112: note = VL sequence
DIVMTQSPLSLPVTPGEPASISCRSSKSLLHSNGITYLYWYLQKPGQSPQLLIYQMSNRASGVPDRFSGSGSGTDFTLKISRVEAEDVGVYYCAQNLELPLTFGQGTKLEIK

SEQ ID NO: 95 - protein, 112 aa, synthetic construct
  REGION 1..112: note = VL sequence
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPLTFGQGTKLEIK

SEQ ID NO: 96 - protein, 112 aa, synthetic construct
  REGION 1..112: note = VL sequence
DIVMTQSPLSLPVTPGEPASISCRSSKSLLHSNGITYLYWYLQKPGQSPQLLIYQMSNLASGVPDRFSSSGSGTDFTLKISRVEAEDVGVYYCAQNLELPLTFGQGTKLEIK

SEQ ID NO: 97 - protein, 112 aa, synthetic construct; identical to SEQ ID NO: 96
  REGION 1..112: note = VL sequence

SEQ ID NO: 98 - protein, 112 aa, synthetic construct; identical to SEQ ID NO: 93
  REGION 1..112: note = VL sequence

SEQ ID NO: 99 - protein, 112 aa, synthetic construct
  REGION 1..112: note = VL sequence
DIVMTQSPLSLPVTPGEPASISCRSSKSLLHSNGNTYLYWFLQKPGQSPQLLIYRLSNLASGVPDRFSGSGSGTDFTLKISRVEAEDVGVYYCMQHLEYPLTFGAGTKLELK

SEQ ID NO: 100 - protein, 112 aa, synthetic construct; identical to SEQ ID NO: 84
  REGION 1..112: note = VL sequence

SEQ ID NO: 101 - protein, 112 aa, synthetic construct; identical to SEQ ID NO: 84
  REGION 1..112: note = VL sequence

SEQ ID NO: 102 - protein, 112 aa, synthetic construct
  REGION 1..112: note = VL sequence
DIVMTQSALSNPVTLGQSASISCRSSKSLLHSQGITYLYWYLQKPGQSPQLLIYQMSNRASGVPDRFSSSGSGTDFTLKISRVEAEDVGVYYCAQNLELPLTFGQGTKLEIK

SEQ ID NO: 103 - protein, 8 aa, synthetic construct
  REGION 1..8: note = HCDR1
GYTFTDYS

SEQ ID NO: 104 - protein, 8 aa, synthetic construct
  REGION 1..8: note = HCDR1
GFTFSRYG

SEQ ID NO: 105 - protein, 11 aa, synthetic construct
  REGION 1..11: note = LCDR1
KSLLHSNGNTY

SEQ ID NO: 106 - protein, 11 aa, synthetic construct
  REGION 1..11: note = LCDR1
KSLLHSSGNTY

SEQ ID NO: 107 - protein, 11 aa, synthetic construct
  REGION 1..11: note = LCDR1
KSLLHSNGITY

SEQ ID NO: 108 - protein, 11 aa, synthetic construct
  REGION 1..11: note = LCDR1
KSLLHSSGITY

SEQ ID NO: 109 - protein, 11 aa, synthetic construct
  REGION 1..11: note = LCDR1
KSLLHSVGITY

SEQ ID NO: 110 - protein, 11 aa, synthetic construct
  REGION 1..11: note = LCDR1
KSLLHSDGITY

SEQ ID NO: 111 - protein, 11 aa, synthetic construct
  REGION 1..11: note = LCDR1
KSLLHSQGITY

SEQ ID NO: 112 - protein, 11 aa, synthetic construct
  REGION 1..11: note = LCDR1
KSLLHSEGITY

SEQ ID NO: 113 - protein, 8 aa, synthetic construct
  REGION 1..8: note = HCDR2
INTETGEP

SEQ ID NO: 114 - protein, 8 aa, synthetic construct
  REGION 1..8: note = HCDR2
IQTETGEP

SEQ ID NO: 115 - protein, 8 aa, synthetic construct
  REGION 1..8: note = HCDR2
ISIGGTYT

SEQ ID NO: 116-123 - skipped (8 entries)

SEQ ID NO: 124 - protein, 7 aa, synthetic construct
  REGION 1..7: note = HCDR3
APGGFAY

SEQ ID NO: 125 - protein, 15 aa, synthetic construct
  REGION 1..15: note = HCDR3
ARRGYGNYSYYGMDY

SEQ ID NO: 126 - protein, 15 aa, synthetic construct
  REGION 1..15: note = HCDR3
ARRGYGSYSYYGMDY

SEQ ID NO: 127 - protein, 15 aa, synthetic construct
  REGION 1..15: note = HCDR3
ARRGYGQYSYYGMDY

SEQ ID NO: 128 - protein, 15 aa, synthetic construct
  REGION 1..15: note = HCDR3
ARRGYGNYSYYGLDY

SEQ ID NO: 129 - protein, 15 aa, synthetic construct
  REGION 1..15: note = HCDR3
ARRGYGNYSYYGIDY

SEQ ID NO: 130 - protein, 15 aa, synthetic construct
  REGION 1..15: note = HCDR3
ARRGYGNYSYYGVDY

SEQ ID NO: 131 - protein, 15 aa, synthetic construct
  REGION 1..15: note = HCDR3
ARRGYGQYSYYGLDY

SEQ ID NO: 132 - protein, 9 aa, synthetic construct
  REGION 1..9: note = LCDR3
MQHLEYPLT

SEQ ID NO: 133 - protein, 9 aa, synthetic construct
  REGION 1..9: note = LCDR3
AQNLELPLT

SEQ ID NO: 134 - protein, 15 aa, synthetic construct
  REGION 1..15: note = HCDR3 consensus sequence
  VARIANT 7: note = Xaa is Asn, Ser or Gln
  VARIANT 13: note = Xaa is Met, Leu, Ile or Val
ARRGYGXYSYYGXDY

SEQ ID NO: 135 - protein, 11 aa, synthetic construct
  REGION 1..11: note = LCDR1 consesnsus sequence
  VARIANT 7: note = Xaa is Asn, Val, Asp or Gln
KSLLHSXGITY

SEQ ID NO: 136 - skipped

SEQ ID NO: 137 - protein, 122 aa, synthetic construct; identical to SEQ ID NO: 16
  REGION 1..122: note = VH of 1D7

SEQ ID NO: 138 - protein, 112 aa, synthetic construct; identical to SEQ ID NO: 67
  REGION 1..112: note = VL of 1D7

SEQ ID NO: 139 - protein, 8 aa, synthetic construct
  REGION 1..8: note = HCDR2 of 1-16 antibodies
  VARIANT 2: note = Xaa is Asn or Gln
IXTETGEP

SEQ ID NO: 140 - protein, 10 aa, synthetic construct
  REGION 1..10: note = LCDR1 of 1-16 antibodies
  VARIANT 7: note = Xaa is Asn or Ser
KSLLHSXGNT

SEQ ID NO: 141 - skipped

SEQ ID NO: 142 - protein, 345 aa, synthetic construct
  REGION 1..345: note = Sec8-His_construct
MRSHHHHHHHHHHRSGTGDDDDKAMADIGSEFELRRQACGFRSRSSSVGSSSSYPISPAVSRTQLASWSDPTEETGPVAGILDTETLEKVSITEAMHRNLVDNITGQRLLEAQACTGGIIDPSTGERFPVTDAVNKGLVDKIMVDRINLAQKAFCGFEDPRTKTKMSAAQALKKGWLYYEAGQRFLEVQYLTGGLIEPDTPGRVPLDEALQRGTVDARTAQKLRDVGAYSKYLTCPKTKLKISYKDALDRSMVEEGTGLRLLEAAAQSTKGYYSPYSVSGSGSTAGSRTGSRTGSRAGSRRGSFDATGSGFSMTFSSSSYSSSGYGRRYASGSSASLGGPESAVA

SEQ ID NO: 143 - protein, 8 aa, synthetic construct
  REGION 1..8: note = VH CDR1
GYTFTDYS

SEQ ID NO: 144 - protein, 8 aa, synthetic construct
  REGION 1..8: note = VH CDR1
GFTFSRYG

SEQ ID NO: 145 - protein, 8 aa, synthetic construct
  REGION 1..8: note = VH CDR1
GFTFSSYS

SEQ ID NO: 146 - protein, 8 aa, synthetic construct
  REGION 1..8: note = VH CDR2
ISTETGEP

SEQ ID NO: 147 - protein, 8 aa, synthetic construct
  REGION 1..8: note = VH CDR2
IVTETGEP

SEQ ID NO: 148 - protein, 8 aa, synthetic construct
  REGION 1..8: note = VH CDR2
IDTETGEP

SEQ ID NO: 149 - protein, 8 aa, synthetic construct
  REGION 1..8: note = VH CDR2
IETETGEP

SEQ ID NO: 150 - protein, 8 aa, synthetic construct
  REGION 1..8: note = VH CDR2
INTETGEP

SEQ ID NO: 151 - protein, 8 aa, synthetic construct
  REGION 1..8: note = VH CDR2
ISIGGTYT

SEQ ID NO: 152 - protein, 8 aa, synthetic construct
  REGION 1..8: note = VH CDR2
ISSSSSYI

SEQ ID NO: 153 - protein, 8 aa, synthetic construct
  REGION 1..8: note = VH CDR2
IQTETGEP

SEQ ID NO: 154 - protein, 7 aa, synthetic construct
  REGION 1..7: note = VH CDR3
APGGFAY

SEQ ID NO: 155 - protein, 7 aa, synthetic construct
  REGION 1..7: note = VH CDR3
ARGGFAY

SEQ ID NO: 156 - protein, 15 aa, synthetic construct
  REGION 1..15: note = VH CDR3
ARRGYGVYSYYGMDY

SEQ ID NO: 157 - protein, 15 aa, synthetic construct
  REGION 1..15: note = VH CDR3
ARRGYGDYSYYGMDY

SEQ ID NO: 158 - protein, 15 aa, synthetic construct
  REGION 1..15: note = VH CDR3
ARRGYGEYSYYGMDY

SEQ ID NO: 159 - protein, 15 aa, synthetic construct
  REGION 1..15: note = VH CDR3
ARRGYGNYSYYGMDY

SEQ ID NO: 160 - protein, 16 aa, synthetic construct
  REGION 1..16: note = VH CDR3
ARRGYGNYSYYGFDYW

SEQ ID NO: 161 - protein, 15 aa, synthetic construct
  REGION 1..15: note = VH CDR3
ARRGYGQYSYYGMDY

SEQ ID NO: 162 - protein, 15 aa, synthetic construct
  REGION 1..15: note = VH CDR3
ARRGYGQYSYYGLDY

SEQ ID NO: 163 - protein, 15 aa, synthetic construct
  REGION 1..15: note = VH CDR3
ARRGYGQYSYYGFDY

SEQ ID NO: 164 - protein, 11 aa, synthetic construct
  REGION 1..11: note = VL CDR1
KSLLHSNGNTY

SEQ ID NO: 165 - protein, 11 aa, synthetic construct
  REGION 1..11: note = VL CDR1
KSLLHSVGNTY

SEQ ID NO: 166 - protein, 11 aa, synthetic construct
  REGION 1..11: note = VL CDR1
KSLLHSDGNTY

SEQ ID NO: 167 - protein, 11 aa, synthetic construct
  REGION 1..11: note = VL CDR1
KSLLHSQGNTY

SEQ ID NO: 168 - protein, 11 aa, synthetic construct
  REGION 1..11: note = VL CDR1
KSLLHSEGNTY

SEQ ID NO: 169 - protein, 11 aa, synthetic construct
  REGION 1..11: note = VL CDR1
KSLLHSNGITY

SEQ ID NO: 170 - protein, 11 aa, synthetic construct
  REGION 1..11: note = VL CDR1
QSLLHSNGYTY

SEQ ID NO: 171 - protein, 11 aa, synthetic construct
  REGION 1..11: note = VL CDR1
QSLLHSNGYNY

SEQ ID NO: 172 - protein, 11 aa, synthetic construct
  REGION 1..11: note = VL CDR1
KSLLHSQGITY

SEQ ID NO: 173 - protein, 11 aa, synthetic construct
  REGION 1..11: note = VL CDR1
QSLLHSQGYNY

SEQ ID NO: 174-178 - skipped (5 entries)

SEQ ID NO: 179 - protein, 9 aa, synthetic construct
  REGION 1..9: note = VL CDR3
MQHLEYPLT

SEQ ID NO: 180 - protein, 9 aa, synthetic construct
  REGION 1..9: note = VL CDR3
AQNLELPLT

SEQ ID NO: 181 - protein, 9 aa, synthetic construct
  REGION 1..9: note = VL CDR2 or CDR3
MQATQFPLT

SEQ ID NO: 182 - protein, 9 aa, synthetic construct
  REGION 1..9: note = VL CDR3
MQALQTPYT

SEQ ID NO: 183 - protein, 9 aa, synthetic construct
  REGION 1..9: note = VL CDR3
MQALQTPLT

SEQ ID NO: 184 - DNA, 14679 bp, Homo sapiens
  source 1..14679: mol_type = unassigned DNA
cccacccgacgctgccgccagtggcttgtgcctcctccgaggggcctgtgacctcccacacccctggcccgctccgtctgccccgtgggctcctgccaccgtccccgatgaagatcgtgcccgatgagcgggatcgtgtgcagaagaaaaccttcaccaagtgggtcaacaagcacctcatcaaggcccagaggcacatcagtgacctgtatgaagacctccgcgatggccacaacctcatctccctgctggaggtcctctcgggggacagcctgccccgggagaaggggaggatgcgtttccacaagctgcagaatgtccagattgccctggactacctccggcaccgccaggtgaagctggtgaacatcaggaatgatgacatcgctgacggcaaccccaagctgacccttggcctcatctggacaatcattctgcacttccagatctcagatatccaggtgagtgggcagtcggaggacatgacggccaaggagaagctgctgctgtggtcgcagcgaatggtggaggggtaccagggcctgcgatgcgacaacttcacctccagctggagagacggccgcctcttcaatgccatcatccaccggcacaagcccctgctcatcgacatgaacaaggtgtaccggcagaccaacctggagaacctggaccaggccttctctgtggcggagcgggacctgggagtgacgcggctcctggaccctgaggacgtggatgtccctcagcccgacgagaagtccatcatcacctacgtctcgtcgctgtatgacgccatgccccgcgtgccggacgtgcaggatggggtgagggccaacgagctgcagctgcgctggcaggagtaccgggagctggtgctgctgctgcttcagtggatgcgacaccacacggccgcctttgaggaacgcaggttcccctccagcttcgaggagattgagatcctgtggtctcagttcctgaagtttaaggagatggagctaccagccaaggaggccgacaagaacaggtccaagggcatctaccaatccctggagggagcggtgcaagcaggccagctcaaggtgccccctggctaccacccgctggatgtggagaaggagtggggcaagctgcacgtggccatcctggagcgggagaagcagctccgcagcgagtttgagaggctggagtgtcttcagcgcatcgtgaccaagctgcagatggaggcggggctgtgtgaggagcagctgaaccaggccgacgccctgctgcagtcggatgtccggctgctggctgcaggcaaagtgccacagcgggcgggggaggtggaacgggacttggacaaggcggatagcatgatccggctgctcttcaacgacgtgcagaccctcaaggatggacggcacccgcagggcgagcagatgtaccgcagggtgtaccgtctgcacgagcgcctggtagccatccgcaccgagtacaacctacggctgaaggcaggcgtggcggcccctgcaacccaggtggcccaggtgactctgcagagtgtgcagaggcgccccgagctggaggactccactctgcgctacctgcaggacctgctggcctgggtggaggagaaccagcaccgtgtggatggcgctgagtggggtgtggacctgcccagcgtggaggcgcagctgggcagccaccgaggcctgcaccagtccatcgaagaattccgggccaagatcgagcgggcacggagtgacgagggccagctctcccccgccacccggggtgcctaccgtgactgcctgggtcggctggacctgcagtacgccaagctgctgaactcctccaaggcccgcctcaggtccctggagagcttgcacagctttgtggcagccgccactaaggagctaatgtggctgaatgagaaggaggaggaggaggtgggcttcgactggagcgaccgcaacaccaacatgaccgccaagaaggagagctactcggcgctgatgcgggagctggagctgaaggagaagaagatcaaggagctccaaaatgctggggaccggctgctgcgggaggaccacccggcccggcccacggtggagtccttccaggcggccctgcagacgcagtggagctggatgctacagctgtgctgctgtatcgaggcacacctgaaggagaacgctgcctactttcagttcttctcagatgtgcgggaggccgaggggcagttgcagaagctgcaggaggcactgcgtaggaaatacagttgtgatcgctccgccaccgtcacccggctggaggacctgctgcaggatgcccaggacgagaaggaacagctgaacgagtacaagggccacctctcaggcctggccaagcgggccaaggccgtcgtgcagctgaagccccgccacccagcccaccccatgcggggccgcctgcccctgctggccgtgtgcgactataagcaggtggaggtgactgtgcacaagggtgacgagtgccagctggtgggccctgcacagccgtcccactggaaggtgctcagcagctccggcagcgaggccgccgtgccctccgtgtgcttcctggtgcccccgcccaaccaggaggcccaggaggccgtcaccaggctggaggcccagcaccaggccctggtcacgctgtggcaccagttgcacgtggacatgaagagccttctggcctggcagagccttcgccgcgacgtgcagctcatccgctcctggtccctggccacgttccgcaccctgaagccagaggagcagcgccaagccctgcacagcctggagctgcactaccaggccttcctgcgggacagccaggacgcgggcggcttcggacccgaggaccggctgatggctgagcgcgagtacggctcctgcagccaccactaccagcagctgctgcagagcctggaacagggtgcacaggaagagtctcgctgccagcgctgcatctccgagctcaaagacatccggctgcagctggaggcctgtgagacgcgcaccgtgcaccgcctgcggctgccgctggacaaagagccggcacgggagtgtgcccagcgcatcgccgagcagcagaaggcacaggcagaggtggaggggctgggcaagggggtcgcccggctctctgccgaggccgagaaggtcttggccctaccagagccatcgcctgcggcccccacgctgcgctcggagctggagctgacgctgggcaagctggagcaggtccgcagcctgtctgccatctacctggagaagctcaagaccatcagcctggtgatccgcggcacgcagggggccgaggaggtgctcagggcccacgaggagcagctcaaggaggcccaggccgtgccggccaccctcccggagctcgaggccaccaaggcctctctgaagaagctgcgggcccaggccgaggcacagcagcccacgttcgacgccctgcgggatgagctgcggggggcacaggaggtgggggagcgactgcagcagcggcacggggagcgggacgtggaggtggagcgctggcgggagcgggtcgcccagttgcttgagcgctggcaggctgtgctggcccagaccgacgtgcggcagcgcgagctcgagcaactgggccgccagctgcgttactaccgcgagagtgcagaccccttgggcgcctggctgcaggacgccaggcggcggcaggagcagatccaggccatgccgctggccgacagccaggctgtgcgggagcagctgcggcaggagcaggccctgctggaggagatcgagcgccacggcgagaaggtcgaggagtgccagaggtttgcgaaacagtacatcaacgccatcaaggactatgaactccagctggtgacgtacaaggcgcagcttgagccggtggcctccccggccaagaagcccaaggtccagtcgggatcagagagtgtcatccaggagtacgtggacctgcgtacgcactacagcgagctgaccacactgacgagccagtacatcaagttcatcagcgagactctgcggcgcatggaggaggaggagaggctggctgagcagcagcgggcagaggagcgcgagcggctggccgaggtggaggccgcgctggagaagcagcggcagctggccgaggcgcacgcccaggcaaaggcacaggcggagcgggaggcgaaggagctgcagcagcgcatgcaggaggaggtggtgcggcgggaggaggcggcggtggacgcgcagcagcagaagcgcagcattcaggaggagctgcagcagctgcggcagagctcggaggcggagatccaggccaaggcccggcaggcagaggcggctgagcgcagccggctgcgcatcgaggaggagatccgcgtggtgcgcctgcagttggaggccaccgagcgccagcgtggcggggctgagggggagctgcaggcactgcgtgcacgggcggaggaggctgaggcacaaaagcgacaggcgcaggaggaggccgagcgcttgcggaggcaggtgcaggacgagagccagcgtaagcggcaggcggaggtggagctggcctcgcgcgtgaaggccgaggccgaggcggcgcgcgagaagcagcgggccctgcaggccctggaggagctgcggctgcaggcggaggaggcggagcggcgcctgcggcaggccgaggtggagcgagcgcggcaggtacaggtggccctggagacggcgcagcgcagtgcagaggcggagctgcagagcaaacgcgcctccttcgccgagaagacggcacagctggagcgctccctgcaggaggaacacgtggctgtggcacagctgcgggaggaggctgagcggcgggcacagcagcaggccgaggccgagcgggcgcgcgaggaggcagagcgggagctggagcgctggcagctcaaggccaacgaggcgctacggctgcggctgcaggcggaggaggtggcgcagcagaagagcctggcgcaggccgaggctgagaagcagaaggaggaggcggagcgcgaggcgcggcggcgcggcaaggcggaggagcaggccgtccggcagcgggagctggctgaacaagagctggagaagcagcggcagctggcggaaggcaccgcgcagcagcgcctggccgcggagcaggagttgatccggctgcgggccgagacggagcagggggagcagcagcggcagctgctggaggaggagctggcccggctgcagcgtgaggcggctgcagccacgcagaaacggcaggagctggaagccgagctggccaaggtgcgggccgagatggaggtgctgctggccagcaaggcgagggctgaggaggagtcgcgctccaccagcgagaagtccaagcagaggctggaggccgaggccggccggttccgcgagctggccgaggaggccgcccgcctgcgtgccctggcggaagaggccaagcggcagcggcagctggccgaggaagacgcggcgcggcagcgggccgaggcggagcgggtgcttgcggagaagctggccgccatcggcgaggccacgcggctcaagacggaggcggagatcgcgctcaaggagaaggaggcggagaacgagcgcctgcggcggctggcggaggacgaggccttccagcggcggcggctggaggagcaggccgcgcaacacaaggctgacatcgaggagcgcctggcccagctgcgcaaggcatcggacagcgagctggagcggcagaaggggctggtggaggacacgctgaggcagcggcggcaggtggaggaggagatcctggcgctgaaggcgagcttcgagaaggcggccgctggcaaggcggagctggagctggagctgggacgcatccgcagcaacgcggaggacacgctgcgcagcaaggagcaggccgagctggaggctgcgaggcagcggcagctggcggcggaggaggagcggcggcgccgtgaggctgaggagcgcgtgcagaagagcctggcggccgaggaggaggccgcacggcagcggaaggcggcgctggaggaagtcgagcggctgaaagccaaggtggaggaggcgcggcgcctgcgggagcgagcggagcaggagtcggcgcggcagctgcagctggcccaggaggccgcccagaagcggctgcaggcggaagagaaggcacacgccttcgcggtgcagcagaaggagcaggagctacagcagacgctgcagcaggagcagagcgtgctggaccagctgcgcggcgaggcggaggcggcccggcgggcggctgaggaggcggaggaggcccgggtgcaggcggagcgtgaggcggcgcagtcccggcggcaggtggaagaggccgagcggctgaagcagtcggcagaggagcaggcacaggcccgggctcaggcacaggcggctgcagagaagctgcgcaaggaggccgagcaagaggcggcgcggcgggcacaggcggagcaggcggccctgcggcagaagcaggcagctgacgcggagatggagaagcataagaaattcgccgagcagacgctgcggcagaaggcgcaggtggagcaggagctgacaacactgcggctgcagctggaggagaccgaccaccagaagaacctgctggacgaggagctgcagcggctgaaggcggaggccacggaggccgcacgccagcgcagccaggtggaggaggagctcttctcggtgcgcgtgcagatggaggagctgagcaagctcaaggcacgcatcgaggctgagaaccgcgcactcatcttgcgtgacaaggacaatacgcagcgcttcctgcaggaggaggctgagaagatgaagcaggtggcggaggaggccgcgcggctgagtgtggcggcccaagaggctgcgcgactgcggcagctggcagaggaggacctggcacagcagcgggccttggcagagaagatgctcaaggagaagatgcaggcggtgcaggaggccacgcgactcaaggctgaggcggaactgctgcagcagcagaaggagcttgcgcaggagcaggcgcggcggctgcaggaggacaaggagcagatggcgcagcagctggcggaggagacgcagggcttccagcggacgctggaggccgagcggcagcggcagctggagatgagcgctgaggctgagcgcctcaagctgcgtgtggccgagatgagccgagcccaggcccgcgctgaggaggacgcccagcgcttccggaagcaggcggaggagatcggtgagaagctgcaccgcacggagctcgccacccaggagaaggtgaccctggtgcagacactggagatccagcgacagcagagtgaccatgatgccgagcgcctgcgggaggccatcgctgagctggagcgtgagaaggagaagctccaacaggaggccaaactgctgcagctcaagtctgaggagatgcagacggtgcagcaggagcagctgctgcaggagacgcaggccctgcagcaaagcttcctctctgaaaaggacagcctgctacagcgggagcgcttcatcgagcaggagaaggccaagctggagcagctcttccaggacgaggtggccaaggcacagcagctgcgtgaggagcagcagcggcagcagcagcagatggagcaggaacggcagcggctggtggccagcatggaggaggcgcggcggcggcagcatgaggccgaggagggcgtgcggcgcaagcaggaggagctgcagcagctggagcagcagcggcggcagcaggaggagctgctggctgaggagaaccagaggctgcgtgagcagctgcagctcctggaggagcagcaccgggccgcgctggcgcactcagaggaggtcactgcctcgcaggtggctgccacaaagaccctgcccaatggccgggatgcacttgatggccccgcggcagaggcagagccggagcacagcttcgatggcctgcggcggaaggtgtcagctcagaggctgcaggaggccggcatcctgagtgcggaggagctgcagcggttggcgcagggccacaccacggtggacgagctcgcacggcgggaagacgtgcgccactacctgcagggccgcagcagtatcgcagggctgttgctgaaggccaccaatgagaagctgagtgtttacgccgccctgcagaggcagctgctgagtcccggcacggccctcatcctgctggaggcgcaggcggcctcaggcttcctgctggaccctgtgcggaaccggcggctgaccgtcaacgaggctgtgaaggagggtgtggtgggccccgagctgcaccacaagctgctgtcggccgagcgcgccgtcactggctacaaggacccctacactggccagcagatctctctcttccaagccatgcagaagggcctcatcgtccgggagcacggcatccgcctgctggaggcccagatcgccacgggcggcgttatcgaccccgtgcacagccaccgcgtgcccgtggacgtggcctaccggcgcggctacttcgacgaggagatgaaccgcgtcctggcggaccccagcgacgacaccaagggcttctttgaccccaacacgcacgagaacctcacgtacctgcagctactggagcgctgcgtggaggaccccgagacgggcctgtgccttctgccactcacggataaggctgccaagggcggggagctggtctacactgactccgaggcccgggacgtctttgagaaggccaccgtgtctgcgccgttcggcaagttccagggcaagacggtgaccatttgggagatcatcaactcggaatacttcacggcagagcagcggcgggacctgctgcggcagttccgcacgggccggatcacagtggagaagatcatcaagatcatcatcacggtggtggaggagcaggagcagaagggccggctttgctttgagggcctgcgcagcctggtgccagccgccgagctgctggagagcagggtcatcgaccgcgagctctaccagcagctgcagcgaggtgagcgctctgtgcgagacgtagccgaggtggacactgtgcggcgggctctccggggtgccaacgtcatcgcgggtgtatggctggaggaggcggggcagaagctgagtatctacaatgccctgaagaaagacctgctgccatccgacatggccgtggccctgttggaagcccaggccggcaccgggcacatcatcgaccccgccaccagcgcccggctgaccgtggacgaggcagtgcgtgctggcctggtgggccccgagtttcatgagaagctgctatcagccgagaaggctgtgacagggtacagggacccctacacagggcagagcgtctccctgttccaggccctgaagaagggcctcattccccgggagcagggcctgcgcctgttggacgcccagctgtccacgggcggcatcgtggaccccagcaagagccaccgcgtgcccctggatgtcgcctgcgcccgaggctgcctggatgaggagaccagcagggccctgtcggcaccaagggccgacgccaaggcctacagtgaccccagcacaggggagccggccacctacggcgagctccagcagcggtgccggcccgaccagctgaccgggctgagcctgctgccgctctcagaaaaggctgctcgggcccggcaggaggagctctactcagagctgcaggcccgtgagacctttgaaaagaccccggttgaggtccccgtgggtggcttcaagggcaggacggtgacggtgtgggagctcatcagctctgagtacttcactgcggagcagcggcaggagctgttgcgtcagttccgcacgggcaaggtcaccgtggagaaggtcatcaagattctcattaccatcgtggaggaggtggagaccctgcggcaggagaggctgtccttcagcggcctccgtgcccctgtgccagccagcgagctcctggcttccggggtcctcagcagagcccagtttgagcagctcaaggacggcaagacgacggtcaaggacctttcggagctgggctccgtgcggacgctgctgcagggcagtggctgcctcgccggcatctacctggaggacaccaaggagaaggtgtccatctacgaggccatgcgccggggcctgctgagagccacaacggctgcgctcctgctggaggcgcaggcggccactggcttcctggtggaccccgtgcggaaccagcgcctgtatgtccacgaggccgtgaaggcgggcgtggtgggccccgagcttcacgagcagctgctgtctgccgagaaggccgtcaccggctacagagacccctactcgggcagcaccatctccctcttccaggccatgcagaagggcctggttctccggcagcacggcatccgcctgctggaggcccagatcgccacgggcggcatcatcgaccccgtgcacagccaccgcgtgcctgtggacgtggcctaccagcgcggctacttcagtgaggagatgaaccgcgtcctggcggaccccagcgacgacaccaagggcttctttgaccccaacacgcatgagaacctcacgtacaggcagctgctggagcggtgcgtggaggaccccgagacgggcttgcgccttctgccactgaaaggggcggagaaggctgaggtggtggagaccacgcaggtgtacactgaggaggagacaagaagggcatttgaagagacacagatcgacattcccggcggcggcagccacggcggctccaccatgtccctgtgggaggtgatgcagtcggacctgatccccgaggagcagcgggcccagctgatggctgacttccaggccggccgggtgaccaaggaacgcatgatcatcatcatcatcgagatcattgagaagacagagatcatccgccagcagggtctggcctcctacgactacgtgcgccgccgcctcacggctgaggacctgttcgaggctcggatcatctctctcgagacctacaacctgctccgggagggcaccaggagcctccgtgaggctctcgaggcggagtccgcctggtgctacctctatggcacgggctccgtggctggtgtctacctgcccggttccaggcagacactgagcatctaccaggctctcaagaaagggctgctgagtgccgaggtggcccgcctgctgctggaggcacaggcagccacaggcttcctgctggacccggtgaagggggagcggctgactgtggatgaggctgtgcggaagggcctcgtggggcccgagctgcacgaccgcctgctctcggctgagcgggcggtcaccggctaccgtgacccctacaccgagcagaccatctcgctcttccaggccatgaagaaggagctgatccctactgaggaggccctgcggctgctggatgcccagctggccaccggcggcatcgtggacccccgcctgggcttccaccttcccctggaggtggcttaccagcgtggctacctcaacaaggacacgcacgaccagctgtcagagcccagcgaggtgcgcagctacgtggacccgtccaccgacgagcgcctcagctacacgcagctgctcaggcggtgccgtcgtgacgacggcaccggccagctgctcctgccactgtcggacgcccgcaagctgaccttccgtggcctgcggaagcagatcaccatggaggagctggtgcgctcgcaggtcatggacgaggccacggcgctgcagctgcgggagggcctgacctccatcgaggaggtcaccaagaacttgcagaagttcctggaaggcaccagctgcatcgctggtgtcttcgtggacgccaccaaggaacggctctcggtgtaccaggccatgaagaagggcatcatccgccccggcacagcctttgagctcctggaggcgcaggcggccaccggttacgtcatcgaccccatcaagggactgaagctgacggtggaggaggctgtgcgtatgggcattgtgggccccgagttcaaggacaagctgctgtcggccgagcgcgccgtcactgggtacaaggacccctactctgggaagctcatctccctcttccaggccatgaagaagggcctgatcctgaaggaccatggcatccgcctgctggaggcccagatcgccacgggcggcatcatcgaccctgaggagagccaccggctgcccgtggaggtggcctacaagcgcggcctcttcgatgaggagatgaacgagatcctgaccgacccctcggacgacaccaagggcttctttgaccctaacacggaggagaacctcacctacctgcagctgatggagcgttgtatcactgacccccagacgggcctgtgtctcttgccgctgaaggagaagaagcgggagcggaagacgtcctccaagtcctccgtgcgcaagcgccgagtggtcatcgtggaccccgagacgggcaaggagatgtcagtgtacgaggcctaccgcaagggcctgattgaccaccagacgtacctggagctgtccgagcaggagtgcgagtgggaggagatcaccatctcctcctcggacggcgtggtcaagtccatgatcatcgaccgccgctccgggcgccagtacgacatcgatgatgccatcgccaagaacctcatcgaccgctcggcactggaccagtaccgcgccggcacgctctccatcaccgagttcgccgacatgctctcgggcaacgccggtggtttccgctcccgttcctcctcggtgggatcctcctcctcctaccccatcagccccgccgtctccaggacccagctggcctcctggtcagaccccactgaggagacgggccccgtggctggcatcctggacacggagacgctggagaaggtgtccatcaccgaggccatgcaccggaacctggtggataacatcacggggcagcggctgctggaggcgcaggcctgcaccgggggcatcatcgaccccagcaccggtgagcgcttccctgtcaccgacgccgtcaacaagggcctggtggacaagatcatggtggaccgcatcaacctggcccagaaggccttctgcggcttcgaggacccacgcaccaagaccaagatgtcggccgcccaggccctgaagaagggctggctctactacgaggccggccagcgcttcctggaggtgcagtacctgaccggcggcttgatcgagcccgacacgccgggccgcgtgcccctggacgaggccctgcagcgcggcacggtggacgcccgcaccgcacagaagctgcgtgacgtgggcgcctactccaagtacctcacctgccctaagaccaagctcaagatctcctataaggacgcgctggaccgcagcatggtggaggagggcacggggctgcggctgctggaggctgccgcgcagtccaccaagggctactacagcccctacagcgtcagcggctccggctctaccgctggctcccgcaccggctcgcgcaccggctcccgggccggctcccgccgcggcagctttgacgccaccggctccggcttctccatgaccttctcttcatcctcctactcctcctcgggctacggccgccgctacgcctcggggtcctcggcctccctggggggccctgagtctgccgtggcctgaggctgcctgcgcccaccccgctctgcatgcggcccagcccggctcccaccgaggcgcgggggccgttttcaacgcttaaaggtgtcttcctcccaagtggtgcctaaagtttaaccaaaaagaccagactaatatattaatatatatctgctgtccagacagcctgtatcttgggggacagggctggcccagccctgctggccgcctcaccccctcgggtctcctcactcccttctacctgccactcacacagccaggtgccttggagggtcccaagctgggccccagcccaccctcctgtcttcccagggtagcccgcctgccagtcctagctgcacagggcagctgggcccaaccctgtctgtagagggccctggtgtttctagcactggcctgcacggtgggccttgctggggacggggggccccagtcagcctctctcccagtctacccagagaagccccttccccatgggaagacgaggccctcgggcccagcccccacagtgctgtctgatctgtgctttccagctcaccccccacactcactcctgagacccctggcctccggcgtcagcctccagcctctgttcccctagtaagtgccttccatgtcggcctctaaccccaggccccgaggacccagacccagtggggaggcggacgttccagccggcatggctgggaactgcagacctgtcctcctggtgggtccaggggcccctccagcttgtggagccccacactggggtgccgcctgcccgtctctctcccatggagccccagccccctttgggcccagggacaccagccaggctctgtgctgaccctcctgttgcacccagccctggtctcagcagcgaccacccctgcctccaccctctgagctttgcatgttccactaaccccgggcgggtggcaggtggaggtgtcaggctgctggcgcctctgcaagggcagaacactaacctgaccgtgggcggggccttgcggtatccgcccccaataaaagcaattccaacctt

SEQ ID NO: 185 - protein, 4515 aa, Homo sapiens
MKIVPDERDRVQKKTFTKWVNKHLIKAQRHISDLYEDLRDGHNLISLLEVLSGDSLPREKGRMRFHKLQNVQIALDYLRHRQVKLVNIRNDDIADGNPKLTLGLIWTIILHFQISDIQVSGQSEDMTAKEKLLLWSQRMVEGYQGLRCDNFTSSWRDGRLFNAIIHRHKPLLIDMNKVYRQTNLENLDQAFSVAERDLGVTRLLDPEDVDVPQPDEKSIITYVSSLYDAMPRVPDVQDGVRANELQLRWQEYRELVLLLLQWMRHHTAAFEERRFPSSFEEIEILWSQFLKFKEMELPAKEADKNRSKGIYQSLEGAVQAGQLKVPPGYHPLDVEKEWGKLHVAILEREKQLRSEFERLECLQRIVTKLQMEAGLCEEQLNQADALLQSDVRLLAAGKVPQRAGEVERDLDKADSMIRLLFNDVQTLKDGRHPQGEQMYRRVYRLHERLVAIRTEYNLRLKAGVAAPATQVAQVTLQSVQRRPELEDSTLRYLQDLLAWVEENQHRVDGAEWGVDLPSVEAQLGSHRGLHQSIEEFRAKIERARSDEGQLSPATRGAYRDCLGRLDLQYAKLLNSSKARLRSLESLHSFVAAATKELMWLNEKEEEEVGFDWSDRNTNMTAKKESYSALMRELELKEKKIKELQNAGDRLLREDHPARPTVESFQAALQTQWSWMLQLCCCIEAHLKENAAYFQFFSDVREAEGQLQKLQEALRRKYSCDRSATVTRLEDLLQDAQDEKEQLNEYKGHLSGLAKRAKAVVQLKPRHPAHPMRGRLPLLAVCDYKQVEVTVHKGDECQLVGPAQPSHWKVLSSSGSEAAVPSVCFLVPPPNQEAQEAVTRLEAQHQALVTLWHQLHVDMKSLLAWQSLRRDVQLIRSWSLATFRTLKPEEQRQALHSLELHYQAFLRDSQDAGGFGPEDRLMAEREYGSCSHHYQQLLQSLEQGAQEESRCQRCISELKDIRLQLEACETRTVHRLRLPLDKEPARECAQRIAEQQKAQAEVEGLGKGVARLSAEAEKVLALPEPSPAAPTLRSELELTLGKLEQVRSLSAIYLEKLKTISLVIRGTQGAEEVLRAHEEQLKEAQAVPATLPELEATKASLKKLRAQAEAQQPTFDALRDELRGAQEVGERLQQRHGERDVEVERWRERVAQLLERWQAVLAQTDVRQRELEQLGRQLRYYRESADPLGAWLQDARRRQEQIQAMPLADSQAVREQLRQEQALLEEIERHGEKVEECQRFAKQYINAIKDYELQLVTYKAQLEPVASPAKKPKVQSGSESVIQEYVDLRTHYSELTTLTSQYIKFISETLRRMEEEERLAEQQRAEERERLAEVEAALEKQRQLAEAHAQAKAQAEREAKELQQRMQEEVVRREEAAVDAQQQKRSIQEELQQLRQSSEAEIQAKARQAEAAERSRLRIEEEIRVVRLQLEATERQRGGAEGELQALRARAEEAEAQKRQAQEEAERLRRQVQDESQRKRQAEVELASRVKAEAEAAREKQRALQALEELRLQAEEAERRLRQAEVERARQVQVALETAQRSAEAELQSKRASFAEKTAQLERSLQEEHVAVAQLREEAERRAQQQAEAERAREEAERELERWQLKANEALRLRLQAEEVAQQKSLAQAEAEKQKEEAEREARRRGKAEEQAVRQRELAEQELEKQRQLAEGTAQQRLAAEQELIRLRAETEQGEQQRQLLEEELARLQREAAAATQKRQELEAELAKVRAEMEVLLASKARAEEESRSTSEKSKQRLEAEAGRFRELAEEAARLRALAEEAKRQRQLAEEDAARQRAEAERVLAEKLAAIGEATRLKTEAEIALKEKEAENERLRRLAEDEAFQRRRLEEQAAQHKADIEERLAQLRKASDSELERQKGLVEDTLRQRRQVEEEILALKASFEKAAAGKAELELELGRIRSNAEDTLRSKEQAELEAARQRQLAAEEERRRREAEERVQKSLAAEEEAARQRKAALEEVERLKAKVEEARRLRERAEQESARQLQLAQEAAQKRLQAEEKAHAFAVQQKEQELQQTLQQEQSVLDQLRGEAEAARRAAEEAEEARVQAEREAAQSRRQVEEAERLKQSAEEQAQARAQAQAAAEKLRKEAEQEAARRAQAEQAALRQKQAADAEMEKHKKFAEQTLRQKAQVEQELTTLRLQLEETDHQKNLLDEELQRLKAEATEAARQRSQVEEELFSVRVQMEELSKLKARIEAENRALILRDKDNTQRFLQEEAEKMKQVAEEAARLSVAAQEAARLRQLAEEDLAQQRALAEKMLKEKMQAVQEATRLKAEAELLQQQKELAQEQARRLQEDKEQMAQQLAEETQGFQRTLEAERQRQLEMSAEAERLKLRVAEMSRAQARAEEDAQRFRKQAEEIGEKLHRTELATQEKVTLVQTLEIQRQQSDHDAERLREAIAELEREKEKLQQEAKLLQLKSEEMQTVQQEQLLQETQALQQSFLSEKDSLLQRERFIEQEKAKLEQLFQDEVAKAQQLREEQQRQQQQMEQERQRLVASMEEARRRQHEAEEGVRRKQEELQQLEQQRRQQEELLAEENQRLREQLQLLEEQHRAALAHSEEVTASQVAATKTLPNGRDALDGPAAEAEPEHSFDGLRRKVSAQRLQEAGILSAEELQRLAQGHTTVDELARREDVRHYLQGRSSIAGLLLKATNEKLSVYAALQRQLLSPGTALILLEAQAASGFLLDPVRNRRLTVNEAVKEGVVGPELHHKLLSAERAVTGYKDPYTGQQISLFQAMQKGLIVREHGIRLLEAQIATGGVIDPVHSHRVPVDVAYRRGYFDEEMNRVLADPSDDTKGFFDPNTHENLTYLQLLERCVEDPETGLCLLPLTDKAAKGGELVYTDSEARDVFEKATVSAPFGKFQGKTVTIWEIINSEYFTAEQRRDLLRQFRTGRITVEKIIKIIITVVEEQEQKGRLCFEGLRSLVPAAELLESRVIDRELYQQLQRGERSVRDVAEVDTVRRALRGANVIAGVWLEEAGQKLSIYNALKKDLLPSDMAVALLEAQAGTGHIIDPATSARLTVDEAVRAGLVGPEFHEKLLSAEKAVTGYRDPYTGQSVSLFQALKKGLIPREQGLRLLDAQLSTGGIVDPSKSHRVPLDVACARGCLDEETSRALSAPRADAKAYSDPSTGEPATYGELQQRCRPDQLTGLSLLPLSEKAARARQEELYSELQARETFEKTPVEVPVGGFKGRTVTVWELISSEYFTAEQRQELLRQFRTGKVTVEKVIKILITIVEEVETLRQERLSFSGLRAPVPASELLASGVLSRAQFEQLKDGKTTVKDLSELGSVRTLLQGSGCLAGIYLEDTKEKVSIYEAMRRGLLRATTAALLLEAQAATGFLVDPVRNQRLYVHEAVKAGVVGPELHEQLLSAEKAVTGYRDPYSGSTISLFQAMQKGLVLRQHGIRLLEAQIATGGIIDPVHSHRVPVDVAYQRGYFSEEMNRVLADPSDDTKGFFDPNTHENLTYRQLLERCVEDPETGLRLLPLKGAEKAEVVETTQVYTEEETRRAFEETQIDIPGGGSHGGSTMSLWEVMQSDLIPEEQRAQLMADFQAGRVTKERMIIIIIEIIEKTEIIRQQGLASYDYVRRRLTAEDLFEARIISLETYNLLREGTRSLREALEAESAWCYLYGTGSVAGVYLPGSRQTLSIYQALKKGLLSAEVARLLLEAQAATGFLLDPVKGERLTVDEAVRKGLVGPELHDRLLSAERAVTGYRDPYTEQTISLFQAMKKELIPTEEALRLLDAQLATGGIVDPRLGFHLPLEVAYQRGYLNKDTHDQLSEPSEVRSYVDPSTDERLSYTQLLRRCRRDDGTGQLLLPLSDARKLTFRGLRKQITMEELVRSQVMDEATALQLREGLTSIEEVTKNLQKFLEGTSCIAGVFVDATKERLSVYQAMKKGIIRPGTAFELLEAQAATGYVIDPIKGLKLTVEEAVRMGIVGPEFKDKLLSAERAVTGYKDPYSGKLISLFQAMKKGLILKDHGIRLLEAQIATGGIIDPEESHRLPVEVAYKRGLFDEEMNEILTDPSDDTKGFFDPNTEENLTYLQLMERCITDPQTGLCLLPLKEKKRERKTSSKSSVRKRRVVIVDPETGKEMSVYEAYRKGLIDHQTYLELSEQECEWEEITISSSDGVVKSMIIDRRSGRQYDIDDAIAKNLIDRSALDQYRAGTLSITEFADMLSGNAGGFRSRSSSVGSSSSYPISPAVSRTQLASWSDPTEETGPVAGILDTETLEKVSITEAMHRNLVDNITGQRLLEAQACTGGIIDPSTGERFPVTDAVNKGLVDKIMVDRINLAQKAFCGFEDPRTKTKMSAAQALKKGWLYYEAGQRFLEVQYLTGGLIEPDTPGRVPLDEALQRGTVDARTAQKLRDVGAYSKYLTCPKTKLKISYKDALDRSMVEEGTGLRLLEAAAQSTKGYYSPYSVSGSGSTAGSRTGSRTGSRAGSRRGSFDATGSGFSMTFSSSSYSSSGYGRRYASGSSASLGGPESAVA

SEQ ID NO: 186 - protein, 451 aa, synthetic construct
  REGION 1..451: note = Ab51 Sequence Heavy Chain
EVQLVESGGGLVQPGGSLRLSCAASGFTFSRYGMSWVRQAPGKRLEWVATISIGGTYTYYPDSMKGRFTISRDNSKNTLYLQMNSLRAEDTAMYYCARRGYGQYSYYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 187 - protein, 219 aa, synthetic construct
  REGION 1..219: note = Ab51 Sequence Light Chain
DIVMTQSALSNPVTLGQSASISCRSSKSLLHSQGITYLYWYLQKPGQSPQLLIYQMSNRASGVPDRFSSSGSGTDFTLKISRVEAEDVGVYYCAQNLELPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 189 - protein, 107 aa, Homo sapiens [sequence omitted]

SEQ ID NO: 190 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 191 - protein, 452 aa, synthetic construct
  REGION 1..452: note = Murine anti-CSP1 IgG1 antibody 1D7 heavy chain
EVQLVESGGDLVKPGGSLKLSCAASGFTFSRYGMSWVRQTPDKRLEWVATISIGGTYTYYPDSMKGRFTISRDNAKNTLYLQMSSLKSEDTAMYYCARRGYGNYSYYGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 219 aa, synthetic construct
  REGION 1..219: note = Murine anti-CSP1 IgG1 antibody 1D7 light chain
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 450 aa, synthetic construct
  REGION 1..450: note = Murine anti-CSP1 IgG2b antibody 1H11HC heavy chain
QIQLVQSGPELKKPGETVKISCKASGYTFTDYSMHWVKQAPGKGLKWMGWINTETGEPTYADDFKGRFAFSLETSASTAYLQINNLKNEDTATYFCAPGGFAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 194 - protein, 219 aa, synthetic construct
  REGION 1..219: note = Murine anti-CSP1 IgG2b antibody 1H11HC light chain
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 195 - protein, 114 aa, synthetic construct
  REGION 1..114: note = VH sequence
QIQLVQSGPELKKPGETVKISCKASGYTFTDYSMHWVKQAPGKGLKWMGWISTETGEPTYADDFKGRFAFSLETSASTAYLQINNLKNEDTATYFCAPGGFAYWGQGTLVTVSA

SEQ ID NO: 196 - protein, 114 aa, synthetic construct
  REGION 1..114: note = VH sequence
QIQLVQSGPELKKPGETVKISCKASGYTFTDYSMHWVKQAPGKGLKWMGWIVTETGEPTYADDFKGRFAFSLETSASTAYLQINNLKNEDTATYFCAPGGFAYWGQGTLVTVSA

SEQ ID NO: 197 - protein, 114 aa, synthetic construct
  REGION 1..114: note = VH sequence
QIQLVQSGPELKKPGETVKISCKASGYTFTDYSMHWVKQAPGKGLKWMGWIDTETGEPTYADDFKGRFAFSLETSASTAYLQINNLKNEDTATYFCAPGGFAYWGQGTLVTVSA

SEQ ID NO: 198 - protein, 114 aa, synthetic construct
  REGION 1..114: note = VH sequence
QIQLVQSGPELKKPGETVKISCKASGYTFTDYSMHWVKQAPGKGLKWMGWIETETGEPTYADDFKGRFAFSLETSASTAYLQINNLKNEDTATYFCAPGGFAYWGQGTLVTVSA

SEQ ID NO: 199 - protein, 114 aa, synthetic construct
  REGION 1..114: note = VH sequence
QIQLVQSGPELKKPGETVKISCKASGYTFTDYSMHWVKQAPGKGLKWMGWINTETGEPTYADDFKGRFAFSLETSASTAYLQINNLKNEDTATYFCARGGFAYWGQGTLVTVSA

SEQ ID NO: 200 - protein, 114 aa, synthetic construct; identical to SEQ ID NO: 1
  REGION 1..114: note = VH sequence

SEQ ID NO: 201 - protein, 114 aa, synthetic construct; identical to SEQ ID NO: 1
  REGION 1..114: note = VH sequence

SEQ ID NO: 202 - protein, 114 aa, synthetic construct; identical to SEQ ID NO: 1
  REGION 1..114: note = VH sequence

SEQ ID NO: 203 - protein, 114 aa, synthetic construct; identical to SEQ ID NO: 1
  REGION 1..114: note = VH sequence

SEQ ID NO: 204 - protein, 114 aa, synthetic construct; identical to SEQ ID NO: 14
  REGION 1..114: note = VH sequence

SEQ ID NO: 205 - protein, 114 aa, synthetic construct
  REGION 1..114: note = VH sequence
EIQLVQSGPGLVQPGGSVRISCAASGYTFTDYSMHWVRQAPGKGLEWMGWINTETGEPTYADSFKGRFTFSLDNSKNTAYLQINSLRAEDTAVYFCAPGGFAYWGQGTLVTVSS

SEQ ID NO: 206 - protein, 114 aa, synthetic construct; identical to SEQ ID NO: 8
  REGION 1..114: note = VH sequence

SEQ ID NO: 207 - protein, 114 aa, synthetic construct; identical to SEQ ID NO: 205
  REGION 1..114: note = VH sequence

SEQ ID NO: 208 - protein, 114 aa, synthetic construct; identical to SEQ ID NO: 205
  REGION 1..114: note = VH sequence

SEQ ID NO: 209 - protein, 122 aa, synthetic construct
  REGION 1..122: note = VH sequence
EVQLVESGGDLVKPGGSLKLSCAASGFTFSRYGMSWVRQTPDKRLEWVATISIGGTYTYYPDSMKGRFTISRDNAKNTLYLQMSSLKSEDTAMYYCARRGYGVYSYYGMDYWGQGTSVTVSS

SEQ ID NO: 210 - protein, 122 aa, synthetic construct
  REGION 1..122: note = VH sequence
EVQLVESGGDLVKPGGSLKLSCAASGFTFSRYGMSWVRQTPDKRLEWVATISIGGTYTYYPDSMKGRFTISRDNAKNTLYLQMSSLKSEDTAMYYCARRGYGDYSYYGMDYWGQGTSVTVSS

SEQ ID NO: 211 - protein, 122 aa, synthetic construct
  REGION 1..122: note = VH sequence
EVQLVESGGDLVKPGGSLKLSCAASGFTFSRYGMSWVRQTPDKRLEWVATISIGGTYTYYPDSMKGRFTISRDNAKNTLYLQMSSLKSEDTAMYYCARRGYGEYSYYGMDYWGQGTSVTVSS

SEQ ID NO: 212 - protein, 122 aa, synthetic construct; identical to SEQ ID NO: 30
  REGION 1..122: note = VH sequence

SEQ ID NO: 213 - protein, 114 aa, synthetic construct; identical to SEQ ID NO: 40
  REGION 1..114: note = VH sequence

SEQ ID NO: 214 - protein, 114 aa, synthetic construct; identical to SEQ ID NO: 40
  REGION 1..114: note = VH sequence

SEQ ID NO: 215 - protein, 114 aa, synthetic construct; identical to SEQ ID NO: 41
  REGION 1..114: note = VH sequence

SEQ ID NO: 216 - protein, 122 aa, synthetic construct; identical to SEQ ID NO: 43
  REGION 1..122: note = VH sequence

SEQ ID NO: 217 - protein, 122 aa, synthetic construct; identical to SEQ ID NO: 46
  REGION 1..122: note = VH sequence

SEQ ID NO: 218 - protein, 122 aa, synthetic construct; identical to SEQ ID NO: 46
  REGION 1..122: note = VH sequence

SEQ ID NO: 219 - protein, 122 aa, synthetic construct; identical to SEQ ID NO: 46
  REGION 1..122: note = VH sequence

SEQ ID NO: 220 - protein, 115 aa, synthetic construct
  REGION 1..115: note = VH sequence
QVQLVQSGAEVKKPGASVKVSCKASGYTFTDYSMHWVRQAPGQGLKWMGWINTETGEPTNYADDFKGRVTMTRDTSISTAYMELSRLRSDDTATYFCAPGGFAYWGQGTLVTVSS

SEQ ID NO: 221 - protein, 115 aa, synthetic construct; identical to SEQ ID NO: 220
  REGION 1..115: note = VH sequence

SEQ ID NO: 222 - protein, 115 aa, synthetic construct; identical to SEQ ID NO: 220
  REGION 1..115: note = VH sequence

SEQ ID NO: 223 - protein, 115 aa, synthetic construct; identical to SEQ ID NO: 220
  REGION 1..115: note = VH sequence

SEQ ID NO: 224 - protein, 115 aa, synthetic construct
  REGION 1..115: note = VH sequence
QVQLVQSGAEVKKPGASVKVSCKASGYTFTDYSMHWVRQAPGQGLKWMGWINTETGEPTNYADDFQGRVTMTRDTSISTAYMELSRLRSDDTATYFCAPGGFAYWGQGTLVTVSS

SEQ ID NO: 225 - protein, 115 aa, synthetic construct; identical to SEQ ID NO: 224
  REGION 1..115: note = VH sequence

SEQ ID NO: 226 - protein, 115 aa, synthetic construct; identical to SEQ ID NO: 224
  REGION 1..115: note = VH sequence

SEQ ID NO: 227 - protein, 115 aa, synthetic construct; identical to SEQ ID NO: 224
  REGION 1..115: note = VH sequence

SEQ ID NO: 228 - protein, 115 aa, synthetic construct
  REGION 1..115: note = VH sequence
QVQLVQSGAEVKKPGASVKVSCKASGYTFTDYSMHWVRQAPGQGLEWMGWINTETGEPTNYADDFKGRVTMTRDTSISTAYMELSRLRSDDTATYFCAPGGFAYWGQGTLVTVSS

SEQ ID NO: 229 - protein, 115 aa, synthetic construct; identical to SEQ ID NO: 228
  REGION 1..115: note = VH sequence

SEQ ID NO: 230 - protein, 115 aa, synthetic construct; identical to SEQ ID NO: 228
  REGION 1..115: note = VH sequence

SEQ ID NO: 231 - protein, 115 aa, synthetic construct; identical to SEQ ID NO: 228
  REGION 1..115: note = VH sequence

SEQ ID NO: 232 - protein, 114 aa, synthetic construct
  REGION 1..114: note = VH sequence
QVQLVQSGAEVKKPGASVKVSCKASGYTFTDYSMHWVRQAPGQGLEWMGWINTETGEPTYADDFQGRVTMTRDTSISTAYMELSRLRSDDTATYFCAPGGFAYWGQGTLVTVSS

SEQ ID NO: 233 - protein, 114 aa, synthetic construct; identical to SEQ ID NO: 232
  REGION 1..114: note = VH sequence

SEQ ID NO: 234 - protein, 114 aa, synthetic construct; identical to SEQ ID NO: 232
  REGION 1..114: note = VH sequence

SEQ ID NO: 235 - protein, 114 aa, synthetic construct; identical to SEQ ID NO: 232
  REGION 1..114: note = VH sequence

SEQ ID NO: 236 - protein, 114 aa, synthetic construct
  REGION 1..114: note = VH sequence
QVQLVQSGAEVKKPGASVKVSCKASGYTFTDYSMHWVRQAPGQGLKWMGWINTETGEPTYADDFKGRVTMTRDTSISTAYMELSRLRSDDTATYFCAPGGFAYWGQGTLVTVSS

SEQ ID NO: 237 - protein, 114 aa, synthetic construct; identical to SEQ ID NO: 236
  REGION 1..114: note = VH sequence

SEQ ID NO: 238 - protein, 114 aa, synthetic construct; identical to SEQ ID NO: 236
  REGION 1..114: note = VH sequence

SEQ ID NO: 239 - protein, 114 aa, synthetic construct; identical to SEQ ID NO: 236
  REGION 1..114: note = VH sequence

SEQ ID NO: 240 - protein, 114 aa, synthetic construct
  REGION 1..114: note = VH sequence
QVQLVQSGAEVKKPGASVKVSCKASGYTFTDYSMHWVRQAPGQGLKWMGWINTETGEPTYADDFQGRVTMTRDTSISTAYMELSRLRSDDTATYFCAPGGFAYWGQGTLVTVSS

SEQ ID NO: 241 - protein, 114 aa, synthetic construct; identical to SEQ ID NO: 240
  REGION 1..114: note = VH sequence

SEQ ID NO: 242 - protein, 114 aa, synthetic construct; identical to SEQ ID NO: 240
  REGION 1..114: note = VH sequence

SEQ ID NO: 243 - protein, 114 aa, synthetic construct; identical to SEQ ID NO: 240
  REGION 1..114: note = VH sequence

SEQ ID NO: 244 - protein, 114 aa, synthetic construct
  REGION 1..114: note = VH sequence
QVQLVQSGAEVKKPGASVKVSCKASGYTFTDYSMHWVRQAPGQGLEWMGWINTETGEPTYADDFKGRVTMTRDTSISTAYMELSRLRSDDTATYFCAPGGFAYWGQGTLVTVSS

SEQ ID NO: 245 - protein, 114 aa, synthetic construct; identical to SEQ ID NO: 244
  REGION 1..114: note = VH sequence

SEQ ID NO: 246 - protein, 114 aa, synthetic construct; identical to SEQ ID NO: 244
  REGION 1..114: note = VH sequence

SEQ ID NO: 247 - protein, 114 aa, synthetic construct; identical to SEQ ID NO: 244
  REGION 1..114: note = VH sequence

SEQ ID NO: 248 - protein, 122 aa, synthetic construct
  REGION 1..122: note = VH sequence
EVQLVESGGGLVKPGGSLRLSCAASGFTFSSYSMSWVRQAPDKGLEWVATISSSSSYIYYPDSVKGRFTISRDNAKNSLYLQMNSLRAEDTAVYYCARRGYGNYSYYGFDYWGQGTLVTVSS

SEQ ID NO: 249 - protein, 122 aa, synthetic construct; identical to SEQ ID NO: 248
  REGION 1..122: note = VH sequence

SEQ ID NO: 250 - protein, 122 aa, synthetic construct
  REGION 1..122: note = VH sequence
EVQLVESGGGLVKPGGSLRLSCAASGFTFSSYSMNWVRQAPDKGLEWVSSISSSSSYIYYPDSVKGRFTISRDNAKNSLYLQMNSLRAEDTAVYYCARRGYGNYSYYGFDYWGQGTLVTVSS

SEQ ID NO: 251 - protein, 122 aa, synthetic construct; identical to SEQ ID NO: 250
  REGION 1..122: note = VH sequence

SEQ ID NO: 252 - protein, 122 aa, synthetic construct
  REGION 1..122: note = VH sequence
EVQLVESGGGLVKPGGSLRLSCAASGFTFSSYSMSWVRQAPDKGLEWVSSISSSSSYIYYADSVKGRFTISRDNAKNSLYLQMNSLRAEDTAVYYCARRGYGNYSYYGFDYWGQGTLVTVSS

SEQ ID NO: 253 - protein, 122 aa, synthetic construct; identical to SEQ ID NO: 252
  REGION 1..122: note = VH sequence

SEQ ID NO: 254 - protein, 114 aa, synthetic construct
  REGION 1..114: note = VH sequence
QIQLVQSGSELKKPGASVKVSCKASGYTFTDYSMHWVRQAPGQGLEWMGWINTETGEPTYAQGFTGRFVFSLDTSVSTAYLQISSLKAEDTAVYFCARGGFAYWGQGTLVTVSS

SEQ ID NO: 255 - protein, 114 aa, synthetic construct; identical to SEQ ID NO: 254
  REGION 1..114: note = VH sequence

SEQ ID NO: 256 - protein, 114 aa, synthetic construct
  REGION 1..114: note = VH sequence
QIQLVQSGSELKKPGASVKVSCKASGYTFTDYSMHWVRQAPGQGLEWMGWIQTETGEPTYAQGFTGRFVFSLDTSVSTAYLQISSLKAEDTAVYFCAPGGFAYWGQGTLVTVSS

SEQ ID NO: 257 - protein, 114 aa, synthetic construct; identical to SEQ ID NO: 256
  REGION 1..114: note = VH sequence

SEQ ID NO: 258 - protein, 114 aa, synthetic construct
  REGION 1..114: note = VH sequence
QIQLVQSGSELKKPGASVKVSCKASGYTFTDYSMHWVRQAPGQGLEWMGWIQTETGEPTYAQGFTGRFVFSLDTSVSTAYLQISSLKAEDTAVYFCARGGFAYWGQGTLVTVSS

SEQ ID NO: 259 - protein, 114 aa, synthetic construct; identical to SEQ ID NO: 258
  REGION 1..114: note = VH sequence

SEQ ID NO: 260 - protein, 114 aa, synthetic construct; identical to SEQ ID NO: 236
  REGION 1..114: note = VH sequence

SEQ ID NO: 261 - protein, 114 aa, synthetic construct; identical to SEQ ID NO: 236
  REGION 1..114: note = VH sequence

SEQ ID NO: 262 - protein, 114 aa, synthetic construct
  REGION 1..114: note = VH sequence
QVQLVQSGAEVKKPGASVKVSCKASGYTFTDYSMHWVRQAPGQGLKWMGWINTETGEPTYADDFKGRVTMTRDTSISTAYMELSRLRSDDTATYFCARGGFAYWGQGTLVTVSS

SEQ ID NO: 263 - protein, 114 aa, synthetic construct; identical to SEQ ID NO: 262
  REGION 1..114: note = VH sequence

SEQ ID NO: 264 - protein, 114 aa, synthetic construct
  REGION 1..114: note = VH sequence
QVQLVQSGAEVKKPGASVKVSCKASGYTFTDYSMHWVRQAPGQGLKWMGWIQTETGEPTYADDFKGRVTMTRDTSISTAYMELSRLRSDDTATYFCAPGGFAYWGQGTLVTVSS

SEQ ID NO: 265 - protein, 114 aa, synthetic construct; identical to SEQ ID NO: 264
  REGION 1..114: note = VH sequence

SEQ ID NO: 266 - protein, 114 aa, synthetic construct
  REGION 1..114: note = VH sequence
QVQLVQSGAEVKKPGASVKVSCKASGYTFTDYSMHWVRQAPGQGLKWMGWIQTETGEPTYADDFKGRVTMTRDTSISTAYMELSRLRSDDTATYFCARGGFAYWGQGTLVTVSS

SEQ ID NO: 267 - protein, 114 aa, synthetic construct; identical to SEQ ID NO: 266
  REGION 1..114: note = VH sequence

SEQ ID NO: 268 - protein, 114 aa, synthetic construct; identical to SEQ ID NO: 232
  REGION 1..114: note = VH sequence

SEQ ID NO: 269 - protein, 114 aa, synthetic construct; identical to SEQ ID NO: 232
  REGION 1..114: note = VH sequence

SEQ ID NO: 270 - protein, 114 aa, synthetic construct
  REGION 1..114: note = VH sequence
QVQLVQSGAEVKKPGASVKVSCKASGYTFTDYSMHWVRQAPGQGLEWMGWINTETGEPTYADDFQGRVTMTRDTSISTAYMELSRLRSDDTATYFCARGGFAYWGQGTLVTVSS

SEQ ID NO: 271 - protein, 114 aa, synthetic construct; identical to SEQ ID NO: 270
  REGION 1..114: note = VH sequence

SEQ ID NO: 272 - protein, 114 aa, synthetic construct
  REGION 1..114: note = VH sequence
QVQLVQSGAEVKKPGASVKVSCKASGYTFTDYSMHWVRQAPGQGLEWMGWIQTETGEPTYADDFQGRVTMTRDTSISTAYMELSRLRSDDTATYFCAPGGFAYWGQGTLVTVSS

SEQ ID NO: 273 - protein, 114 aa, synthetic construct; identical to SEQ ID NO: 272
  REGION 1..114: note = VH sequence

SEQ ID NO: 274 - protein, 114 aa, synthetic construct
  REGION 1..114: note = VH sequence
QVQLVQSGAEVKKPGASVKVSCKASGYTFTDYSMHWVRQAPGQGLEWMGWIQTETGEPTYADDFQGRVTMTRDTSISTAYMELSRLRSDDTATYFCARGGFAYWGQGTLVTVSS

SEQ ID NO: 275 - protein, 114 aa, synthetic construct; identical to SEQ ID NO: 274
  REGION 1..114: note = VH sequence

SEQ ID NO: 276 - protein, 114 aa, synthetic construct; identical to SEQ ID NO: 236
  REGION 1..114: note = VH sequence

SEQ ID NO: 277 - protein, 114 aa, synthetic construct; identical to SEQ ID NO: 262
  REGION 1..114: note = VH sequence

SEQ ID NO: 278 - protein, 114 aa, synthetic construct; identical to SEQ ID NO: 264
  REGION 1..114: note = VH sequence

SEQ ID NO: 279 - protein, 114 aa, synthetic construct; identical to SEQ ID NO: 266
  REGION 1..114: note = VH sequence

SEQ ID NO: 280 - protein, 114 aa, synthetic construct; identical to SEQ ID NO: 232
  REGION 1..114: note = VH sequence

SEQ ID NO: 281 - protein, 114 aa, synthetic construct; identical to SEQ ID NO: 270
  REGION 1..114: note = VH sequence

SEQ ID NO: 282 - protein, 114 aa, synthetic construct; identical to SEQ ID NO: 272
  REGION 1..114: note = VH sequence

SEQ ID NO: 283 - protein, 114 aa, synthetic construct; identical to SEQ ID NO: 274
  REGION 1..114: note = VH sequence

SEQ ID NO: 284 - protein, 122 aa, synthetic construct
  REGION 1..122: note = VH sequence
EVQLVESGGGLVKPGGSLRLSCAASGFTFSRYGMSWVRQAPGKGLEWVSSISIGGTYTYYADSVKGRFTISRDNAKNSLYLQMNSLRAEDTAVYYCARRGYGQYSYYGMDYWGQGTLVTVSS

SEQ ID NO: 285 - protein, 122 aa, synthetic construct; identical to SEQ ID NO: 284
  REGION 1..122: note = VH sequence

SEQ ID NO: 286 - protein, 122 aa, synthetic construct
  REGION 1..122: note = VH sequence
EVQLVESGGGLVKPGGSLRLSCAASGFTFSRYGMSWVRQAPGKGLEWVSSISIGGTYTYYADSVKGRFTISRDNAKNSLYLQMNSLRAEDTAVYYCARRGYGQYSYYGLDYWGQGTLVTVSS

SEQ ID NO: 287 - protein, 122 aa, synthetic construct; identical to SEQ ID NO: 286
  REGION 1..122: note = VH sequence

SEQ ID NO: 288 - protein, 122 aa, synthetic construct
  REGION 1..122: note = VH sequence
EVQLVESGGGLVKPGGSLRLSCAASGFTFSSYSMSWVRQAPDKGLEWVSSISSSSSYIYYADSVKGRFTISRDNAKNSLYLQMNSLRAEDTAVYYCARRGYGQYSYYGFDYWGQGTLVTVSS

SEQ ID NO: 289 - protein, 122 aa, synthetic construct; identical to SEQ ID NO: 288
  REGION 1..122: note = VH sequence

SEQ ID NO: 290 - protein, 122 aa, synthetic construct
  REGION 1..122: note = VH sequence
EVQLVESGGGLVKPGGSLRLSCAASGFTFSSYSMSWVRQAPDKGLEWVSSISSSSSYIYYADSVKGRFTISRDNAKNSLYLQMNSLRAEDTAVYYCARRGYGQYSYYGLDYWGQGTLVTVSS

SEQ ID NO: 291 - protein, 122 aa, synthetic construct; identical to SEQ ID NO: 290
  REGION 1..122: note = VH sequence

SEQ ID NO: 292 - protein, 122 aa, synthetic construct; identical to SEQ ID NO: 49
  REGION 1..122: note = VH sequence

SEQ ID NO: 293 - protein, 122 aa, synthetic construct
  REGION 1..122: note = VH sequence
EVQLVESGGGLVKPGGSLRLSCAASGFTFSSYSMSWVRQAPDKGLEWVSSISSSSSYIYYADSVKGRFTISRDNAKNSLYLQMNSLRAEDTAVYYCARRGYGQYSYYGMDYWGQGTLVTVSS

SEQ ID NO: 294 - protein, 122 aa, synthetic construct; identical to SEQ ID NO: 293
  REGION 1..122: note = VH sequence

SEQ ID NO: 295 - protein, 112 aa, synthetic construct; identical to SEQ ID NO: 52
  REGION 1..112: note = VL sequence

SEQ ID NO: 296 - protein, 112 aa, synthetic construct; identical to SEQ ID NO: 52
  REGION 1..112: note = VL sequence

SEQ ID NO: 297 - protein, 112 aa, synthetic construct; identical to SEQ ID NO: 52
  REGION 1..112: note = VL sequence

SEQ ID NO: 298 - protein, 112 aa, synthetic construct; identical to SEQ ID NO: 52
  REGION 1..112: note = VL sequence

SEQ ID NO: 299 - protein, 112 aa, synthetic construct; identical to SEQ ID NO: 52
  REGION 1..112: note = VL sequence

SEQ ID NO: 300 - protein, 112 aa, synthetic construct
  REGION 1..112: note = VL sequence
DIVMTQAAPSVPVTPGESVSISCRSSKSLLHSVGNTYLYWFLQRPGQSPQLLIYRMSNLASGVPDRFSGSGSGTAFTLRISRVEAEDVGVYYCMQHLEYPLTFGAGTKLELK

SEQ ID NO: 301 - protein, 112 aa, synthetic construct
  REGION 1..112: note = VL sequence
DIVMTQAAPSVPVTPGESVSISCRSSKSLLHSDGNTYLYWFLQRPGQSPQLLIYRMSNLASGVPDRFSGSGSGTAFTLRISRVEAEDVGVYYCMQHLEYPLTFGAGTKLELK

SEQ ID NO: 302 - protein, 112 aa, synthetic construct
  REGION 1..112: note = VL sequence
DIVMTQAAPSVPVTPGESVSISCRSSKSLLHSQGNTYLYWFLQRPGQSPQLLIYRMSNLASGVPDRFSGSGSGTAFTLRISRVEAEDVGVYYCMQHLEYPLTFGAGTKLELK

SEQ ID NO: 303 - protein, 112 aa, synthetic construct
  REGION 1..112: note = VL sequence
DIVMTQAAPSVPVTPGESVSISCRSSKSLLHSEGNTYLYWFLQRPGQSPQLLIYRMSNLASGVPDRFSGSGSGTAFTLRISRVEAEDVGVYYCMQHLEYPLTFGAGTKLELK

SEQ ID NO: 304 - protein, 112 aa, synthetic construct; identical to SEQ ID NO: 52
  REGION 1..112: note = VL sequence

SEQ ID NO: 305 - protein, 112 aa, synthetic construct; identical to SEQ ID NO: 52
  REGION 1..112: note = VL sequence

SEQ ID NO: 306 - protein, 112 aa, synthetic construct; identical to SEQ ID NO: 61
  REGION 1..112: note = VL sequence

SEQ ID NO: 307 - protein, 112 aa, synthetic construct; identical to SEQ ID NO: 60
  REGION 1..112: note = VL sequence

SEQ ID NO: 308 - protein, 112 aa, synthetic construct; identical to SEQ ID NO: 61
  REGION 1..112: note = VL sequence

SEQ ID NO: 309 - protein, 112 aa, synthetic construct; identical to SEQ ID NO: 67
  REGION 1..112: note = VL sequence

SEQ ID NO: 310 - protein, 112 aa, synthetic construct; identical to SEQ ID NO: 67
  REGION 1..112: note = VL sequence

SEQ ID NO: 311 - protein, 112 aa, synthetic construct; identical to SEQ ID NO: 67
  REGION 1..112: note = VL sequence

SEQ ID NO: 312 - protein, 112 aa, synthetic construct; identical to SEQ ID NO: 85
  REGION 1..112: note = VL sequence

SEQ ID NO: 313 - protein, 112 aa, synthetic construct
  REGION 1..112: note = VL sequence
DIVMTQSPLSLPVTPGEPASISCRSSKSLLHSNGNTYLDWYLQKPGQSPQLLIYRMSNRASGVPDRFSGSGSGTDFTLKISRVEAEDVGVYYCMQHLEYPLTFGQGTKLEIK

SEQ ID NO: 314 - protein, 112 aa, synthetic construct; identical to SEQ ID NO: 93
  REGION 1..112: note = VL sequence

SEQ ID NO: 315 - protein, 112 aa, synthetic construct; identical to SEQ ID NO: 313
  REGION 1..112: note = VL sequence

SEQ ID NO: 316 - protein, 112 aa, synthetic construct
  REGION 1..112: note = VL sequence
DIVMTQSPLSLPVTPGEPASISCRSSKSLLHSNGITYLDWYLQKPGQSPQLLIYQMSNRASGVPDRFSGSGSGTDFTLKISRVEAEDVGVYYCAQNLELPLTFGQGTKLEIK

SEQ ID NO: 317 - protein, 112 aa, synthetic construct; identical to SEQ ID NO: 316
  REGION 1..112: note = VL sequence

SEQ ID NO: 318 - protein, 112 aa, synthetic construct; identical to SEQ ID NO: 94
  REGION 1..112: note = VL sequence

SEQ ID NO: 319 - protein, 112 aa, synthetic construct; identical to SEQ ID NO: 95
  REGION 1..112: note = VL sequence

SEQ ID NO: 320 - protein, 112 aa, synthetic construct
  REGION 1..112: note = VL sequence
DIVMTQTPPSLPVNPGEPASISCRSSQSLLHSNGYTYLYWYLQKPGQSPQLLIYRVSNLASGVPDRFSGSGSGSDFTLKISWVEAEDVGVYYCMQATQFPLTFGQGTKLEIK

SEQ ID NO: 321 - protein, 112 aa, synthetic construct
  REGION 1..112: note = VL sequence
DIVMTQTPPSLPVNPGEPASISCRSSQSLLHSNGYTYLYWYLQKPGQSPQLLIYRVSNHLSGVPDRFSGSGSGSDFTLKISWVEAEDVGVYYCMQATQFPLTFGAGTKLEIK

SEQ ID NO: 322 - protein, 112 aa, synthetic construct
  REGION 1..112: note = VL sequence
DIVMTQTPPSLPVNPGEPASISCRSSQSLLHSNGYTYLYWYLQKPGQSPQLLIYRVSNLLSGVPDRFSGSGSGSDFTLKISWVEAEDVGVYYCMQATQFPLTFGQGTKLEIK

SEQ ID NO: 323 - protein, 112 aa, synthetic construct
  REGION 1..112: note = VL sequence
DIVMTQTPPSLPVNPGEPASISCRSSQSLLHSNGYTYLYWFLQKPGQSPQLLIYRVSNLASGVPDRFSGSGSGSDFTLKISWVEAEDVGVYYCMQATQFPLTFGAGTKLEIK

SEQ ID NO: 324 - protein, 112 aa, synthetic construct; identical to SEQ ID NO: 320
  REGION 1..112: note = VL sequence

SEQ ID NO: 325 - protein, 112 aa, synthetic construct; identical to SEQ ID NO: 321
  REGION 1..112: note = VL sequence

SEQ ID NO: 326 - protein, 112 aa, synthetic construct; identical to SEQ ID NO: 322
  REGION 1..112: note = VL sequence

SEQ ID NO: 327 - protein, 112 aa, synthetic construct; identical to SEQ ID NO: 323
  REGION 1..112: note = VL sequence

SEQ ID NO: 328 - protein, 112 aa, synthetic construct; identical to SEQ ID NO: 320
  REGION 1..112: note = VL sequence

SEQ ID NO: 329 - protein, 112 aa, synthetic construct; identical to SEQ ID NO: 321
  REGION 1..112: note = VL sequence

SEQ ID NO: 330 - protein, 112 aa, synthetic construct; identical to SEQ ID NO: 322
  REGION 1..112: note = VL sequence

SEQ ID NO: 331 - protein, 112 aa, synthetic construct; identical to SEQ ID NO: 323
  REGION 1..112: note = VL sequence

SEQ ID NO: 332 - protein, 112 aa, synthetic construct; identical to SEQ ID NO: 320
  REGION 1..112: note = VL sequence

SEQ ID NO: 333 - protein, 112 aa, synthetic construct; identical to SEQ ID NO: 321
  REGION 1..112: note = VL sequence

SEQ ID NO: 334 - protein, 112 aa, synthetic construct; identical to SEQ ID NO: 322
  REGION 1..112: note = VL sequence

SEQ ID NO: 335 - protein, 112 aa, synthetic construct; identical to SEQ ID NO: 323
  REGION 1..112: note = VL sequence

SEQ ID NO: 336 - protein, 112 aa, synthetic construct; identical to SEQ ID NO: 320
  REGION 1..112: note = VL sequence

SEQ ID NO: 337 - protein, 112 aa, synthetic construct; identical to SEQ ID NO: 321
  REGION 1..112: note = VL sequence

SEQ ID NO: 338 - protein, 112 aa, synthetic construct; identical to SEQ ID NO: 322
  REGION 1..112: note = VL sequence

SEQ ID NO: 339 - protein, 112 aa, synthetic construct; identical to SEQ ID NO: 323
  REGION 1..112: note = VL sequence

SEQ ID NO: 340 - protein, 112 aa, synthetic construct; identical to SEQ ID NO: 320
  REGION 1..112: note = VL sequence

SEQ ID NO: 341 - protein, 112 aa, synthetic construct; identical to SEQ ID NO: 321
  REGION 1..112: note = VL sequence

SEQ ID NO: 342 - protein, 112 aa, synthetic construct; identical to SEQ ID NO: 322
  REGION 1..112: note = VL sequence

SEQ ID NO: 343 - protein, 112 aa, synthetic construct; identical to SEQ ID NO: 323
  REGION 1..112: note = VL sequence

SEQ ID NO: 344 - protein, 112 aa, synthetic construct; identical to SEQ ID NO: 320
  REGION 1..112: note = VL sequence

SEQ ID NO: 345 - protein, 112 aa, synthetic construct; identical to SEQ ID NO: 321
  REGION 1..112: note = VL sequence

SEQ ID NO: 346 - protein, 112 aa, synthetic construct; identical to SEQ ID NO: 322
  REGION 1..112: note = VL sequence

SEQ ID NO: 347 - protein, 112 aa, synthetic construct; identical to SEQ ID NO: 323
  REGION 1..112: note = VL sequence

SEQ ID NO: 348 - protein, 112 aa, synthetic construct
  REGION 1..112: note = VL sequence
DIVMTQSPLSLPVTPGEPASISCRSSQSLLHSNGYNYLYWYLQKPGQSPQLLIYLGSNLASGVPDRFSGSGSGTDFTLKISRVEAEDVGVYYCMQALQTPYTFGQGTKLEIK

SEQ ID NO: 349 - protein, 112 aa, synthetic construct
  REGION 1..112: note = VL sequence
DIVMTQSPLSLPVTPGEPASISCRSSQSLLHSNGYNYLYWYLQKPGQSPQLLIYLGSNLASGVPDRFSGSGSGTDFTLKISRVEAEDVGVYYCMQALQTPLTFGQGTKLEIK

SEQ ID NO: 350 - protein, 112 aa, synthetic construct; identical to SEQ ID NO: 348
  REGION 1..112: note = VL sequence

SEQ ID NO: 351 - protein, 112 aa, synthetic construct; identical to SEQ ID NO: 349
  REGION 1..112: note = VL sequence

SEQ ID NO: 352 - protein, 112 aa, synthetic construct; identical to SEQ ID NO: 348
  REGION 1..112: note = VL sequence

SEQ ID NO: 353 - protein, 112 aa, synthetic construct; identical to SEQ ID NO: 349
  REGION 1..112: note = VL sequence

SEQ ID NO: 354 - protein, 112 aa, synthetic construct; identical to SEQ ID NO: 93
  REGION 1..112: note = VL sequence

SEQ ID NO: 355 - protein, 112 aa, synthetic construct; identical to SEQ ID NO: 99
  REGION 1..112: note = VL sequence

SEQ ID NO: 356 - protein, 112 aa, synthetic construct; identical to SEQ ID NO: 93
  REGION 1..112: note = VL sequence

SEQ ID NO: 357 - protein, 112 aa, synthetic construct; identical to SEQ ID NO: 99
  REGION 1..112: note = VL sequence

SEQ ID NO: 358 - protein, 112 aa, synthetic construct; identical to SEQ ID NO: 93
  REGION 1..112: note = VL sequence

SEQ ID NO: 359 - protein, 112 aa, synthetic construct; identical to SEQ ID NO: 99
  REGION 1..112: note = VL sequence

SEQ ID NO: 360 - protein, 112 aa, synthetic construct; identical to SEQ ID NO: 321
  REGION 1..112: note = VL sequence

SEQ ID NO: 361 - protein, 112 aa, synthetic construct
  REGION 1..112: note = VL sequence
DIVMTQTPPSLPVNPGEPASISCRSSQSLLHSNGYTYLYWYLQKPGQSPQLLIYRLSNHLSGVPDRFSGSGSGSDFTLKISWVEAEDVGVYYCMQATQFPLTFGAGTKLEIK

SEQ ID NO: 362 - protein, 112 aa, synthetic construct; identical to SEQ ID NO: 321
  REGION 1..112: note = VL sequence

SEQ ID NO: 363 - protein, 112 aa, synthetic construct; identical to SEQ ID NO: 361
  REGION 1..112: note = VL sequence

SEQ ID NO: 364 - protein, 112 aa, synthetic construct; identical to SEQ ID NO: 321
  REGION 1..112: note = VL sequence

SEQ ID NO: 365 - protein, 112 aa, synthetic construct; identical to SEQ ID NO: 361
  REGION 1..112: note = VL sequence

SEQ ID NO: 366 - protein, 112 aa, synthetic construct; identical to SEQ ID NO: 321
  REGION 1..112: note = VL sequence

SEQ ID NO: 367 - protein, 112 aa, synthetic construct; identical to SEQ ID NO: 361
  REGION 1..112: note = VL sequence

SEQ ID NO: 368 - protein, 112 aa, synthetic construct; identical to SEQ ID NO: 320
  REGION 1..112: note = VL sequence

SEQ ID NO: 369 - protein, 112 aa, synthetic construct
  REGION 1..112: note = VL sequence
DIVMTQTPPSLPVNPGEPASISCRSSQSLLHSNGYTYLYWYLQKPGQSPQLLIYRLSNLASGVPDRFSGSGSGSDFTLKISWVEAEDVGVYYCMQATQFPLTFGQGTKLEIK

SEQ ID NO: 370 - protein, 112 aa, synthetic construct; identical to SEQ ID NO: 320
  REGION 1..112: note = VL sequence

SEQ ID NO: 371 - protein, 112 aa, synthetic construct; identical to SEQ ID NO: 369
  REGION 1..112: note = VL sequence

SEQ ID NO: 372 - protein, 112 aa, synthetic construct
  REGION 1..112: note = VL sequence
DIVMTQTPPSLPVNPGEPASISCRSSQSLLHSNGYTYLYWYLQKPGQSPQLLIYNLASGVPDRFSGSGSGSDFTLKISWVEAEDVGVYYCMQATQFPLTFRVSGQGTKLEIK

SEQ ID NO: 373 - protein, 112 aa, synthetic construct; identical to SEQ ID NO: 369
  REGION 1..112: note = VL sequence

SEQ ID NO: 374 - protein, 112 aa, synthetic construct; identical to SEQ ID NO: 320
  REGION 1..112: note = VL sequence

SEQ ID NO: 375 - protein, 112 aa, synthetic construct; identical to SEQ ID NO: 369
  REGION 1..112: note = VL sequence

SEQ ID NO: 376 - protein, 112 aa, synthetic construct
  REGION 1..112: note = VL sequence
DIVMTQTPPSLPVNPGEPASISCRSSQSLLHSNGYTYLYWYLQKPGQSPQLLIYRMSNHLSGVPDRFSGSGSGSDFTLKISWVEAEDVGVYYCMQATQFPLTFGAGTKLEIK

SEQ ID NO: 377 - protein, 112 aa, synthetic construct; identical to SEQ ID NO: 376
  REGION 1..112: note = VL sequence

SEQ ID NO: 378 - protein, 112 aa, synthetic construct; identical to SEQ ID NO: 376
  REGION 1..112: note = VL sequence

SEQ ID NO: 379 - protein, 112 aa, synthetic construct; identical to SEQ ID NO: 376
  REGION 1..112: note = VL sequence

SEQ ID NO: 380 - protein, 112 aa, synthetic construct
  REGION 1..112: note = VL sequence
DIVMTQTPPSLPVNPGEPASISCRSSQSLLHSNGYTYLYWYLQKPGQSPQLLIYRMSNLASGVPDRFSGSGSGSDFTLKISWVEAEDVGVYYCMQATQFPLTFGQGTKLEIK

SEQ ID NO: 381 - protein, 112 aa, synthetic construct; identical to SEQ ID NO: 380
  REGION 1..112: note = VL sequence

SEQ ID NO: 382 - protein, 112 aa, synthetic construct; identical to SEQ ID NO: 380
  REGION 1..112: note = VL sequence

SEQ ID NO: 383 - protein, 112 aa, synthetic construct; identical to SEQ ID NO: 380
  REGION 1..112: note = VL sequence

SEQ ID NO: 384 - protein, 112 aa, synthetic construct; identical to SEQ ID NO: 95
  REGION 1..112: note = VL sequence

SEQ ID NO: 385 - protein, 112 aa, synthetic construct
  REGION 1..112: note = VL sequence
DIVMTQSPLSLPVTPGEPASISCRSSKSLLHSQGITYLYWYLQKPGQSPQLLIYQMSNLASGVPDRFSGSGSGTDFTLKISRVEAEDVGVYYCAQNLELPLTFGQGTKLEIK

SEQ ID NO: 386 - protein, 112 aa, synthetic construct; identical to SEQ ID NO: 95
  REGION 1..112: note = VL sequence

SEQ ID NO: 387 - protein, 112 aa, synthetic construct; identical to SEQ ID NO: 385
  REGION 1..112: note = VL sequence

SEQ ID NO: 388 - protein, 112 aa, synthetic construct; identical to SEQ ID NO: 348
  REGION 1..112: note = VL sequence

SEQ ID NO: 389 - protein, 112 aa, synthetic construct
  REGION 1..112: note = VL sequence
DIVMTQSPLSLPVTPGEPASISCRSSQSLLHSQGYNYLYWYLQKPGQSPQLLIYLGSNLASGVPDRFSGSGSGTDFTLKISRVEAEDVGVYYCMQALQTPYTFGQGTKLEIK

SEQ ID NO: 390 - protein, 112 aa, synthetic construct; identical to SEQ ID NO: 348
  REGION 1..112: note = VL sequence

SEQ ID NO: 391 - protein, 112 aa, synthetic construct; identical to SEQ ID NO: 389
  REGION 1..112: note = VL sequence

SEQ ID NO: 392 - protein, 112 aa, synthetic construct; identical to SEQ ID NO: 102
  REGION 1..112: note = VL sequence

SEQ ID NO: 393 - protein, 112 aa, synthetic construct; identical to SEQ ID NO: 348
  REGION 1..112: note = VL sequence

SEQ ID NO: 394 - protein, 112 aa, synthetic construct; identical to SEQ ID NO: 389
  REGION 1..112: note = VL sequence

SEQ ID NO: 395 - protein, 215 aa, synthetic construct
MGHHHHHHSHMQLASWSDPTEETGPVAGILDTETLEKVSITEAMHRNLVDNITGQRLLEAQACTGGIIDPSTGERFPVTDAVNKGLVDKIMVDRINLAQKAFCGFEDPRTKTKMSAAQALKKGWLYYEAGQRFLEVQYLTGGLIEPDTPGRVPLDEALQRGTVDARTAQKLRDVGAYSKYLTCPKTKLKISYKDALDRSMVEEGTGLRLLEAAAQ